(19) United States
(12) Patent Application Publication
(10) Pub. No.: US 2025/0276081 A1
(43) Pub. Date: Sep. 4, 2025

(54) Mucin-Active Proteases and Methods of Use

(71) Applicant: The Board of Trustees of the Leland Stanford Junior University, Stanford, CA (US)

(72) Inventors: Carolyn R. Bertozzi, Menlo Park, CA (US); Kayvon Pedram, Redwood City, CA (US); Dayeon Shon, Redwood City, CA (US); Gabrielle Tender, Redwood City, CA (US)

(21) Appl. No.: 18/859,437

(22) PCT Filed: Apr. 28, 2023
(86) PCT No.: PCT/US2023/066410

§ 371 Date: Oct. 23, 2024

Related U.S. Application Data
(60) Provisional application No. 63/336,974, filed on Apr. 29, 2022.

(51) Int. Cl. A61K 47/68; A61P 35/00; C07K 16/32; C12N 9/50

(52) U.S. Cl. CPC A61K 47/6815; A61K 47/6855; A61P 35/00; C07K 16/32; C12N 9/50; C07K 2317/569

(57) ABSTRACT

Provided are mucin-active proteases. In certain embodiments, the mucin-active proteases are stably associated with a targeting moiety. According to some embodiments, the mucin-active protease is stably associated with the targeting moiety via fusion of a protein domain comprising the mucin-active protease and a protein domain comprising the targeting moiety. In other embodiments, the mucin-active protease is stably associated with the targeting moiety via conjugation. Also provided are methods of treating a mucin-associated condition in a subject in need thereof, such methods comprising administering to the subject an effective amount of a mucin-active protease of the present disclosure. Upon administration of the mucin-active protease to the subject, the targeting moiety targets the mucin-active protease to cell surface, extracellular and/or secreted mucins, and the mucin-active protease degrades the mucins.

### CROSS-REFERENCE TO RELATED APPLICATIONS

This application claims the benefit of U.S. Provisional Patent Application No. 63/336,974, filed Apr. 29, 2022, which application is incorporated herein by reference in its entirety.

### STATEMENT OF GOVERNMENT SUPPORT

This invention was made with Government support under contract CA227942 awarded by the National Institutes of Health. The Government has certain rights in the invention.

### INCORPORATION BY REFERENCE OF SEQUENCE LISTING PROVIDED AS A SEQUENCE LISTING XLM FILE

A Sequence Listing is provided herewith as a Sequence Listing XML, STAN-1929WO_SEQ_LIST, created on Apr. 28, 2023 and having a size of 31,074 bytes. The contents of the Sequence Listing XML are incorporated herein by reference in their entirety.

### INTRODUCTION

Glycosylation is the enzymatic post-translational addition of carbohydrates (glycans) to proteins and lipids, resulting in “glycoproteins” and “glycolipids,” respectively. Canonically, glycoprotein glycans can be N-linked (linkage to the amide group of Asn) or O-linked (linkage to the hydroxyl group of Ser, Thr). The particular glycan structures, the “glycoforms,” of a glycoprotein impact the function, stability, folding, localization and ligand specificity of the glycoprotein, and play a role in cell adhesion and cell trafficking by modulating how cells interact with each other and with their extracellular matrix environment. The regular process of glycosylation is disrupted during malignant transformation of cells leading to the abnormal, aberrant expression of glycans, that can manifest by, e.g., altered branching and/or truncation of the glycan structures. Aberrantly expressed glycan structures play a role in the pathogenesis and metastasis of solid cancers and hematological cancers.

Mucins are glycoproteins that bear a high density of O-glycosylated serine and threonine residues. In species ranging from sea sponges to mammals, they are expressed at epithelial and endothelial surfaces, where they defend against physical and biotic threats¹. Mucin domains are modular protein domains that adopt rigid and extended bottle-brush like structures due to a high density of O-glycosylated serine and threonine residues. Mucin-type O-glycans are characterized by an initiating α-N-acetylgalactosamine (α-GalNAc) monosaccharide that can be further elaborated into several core structures through complex regulation of glycosyltransferases. As a result, mucin domains serve as highly heterogenous glycoproteins that exert both biophysical and biochemical influence. For instance, this includes the ability to redistribute receptor molecules at the cell surface and extracellular space and to drive high avidity binding interactions. In the canonical mucin (MUC) family, mucin domains occur as tandem repeats, creating heavily glycosylated superstructures. Canonical mucins are central to many functions in health and disease, and have long been associated with human cancers, e.g., MUC1 and MUC16 (also known as CA-125). Dysregulation of mucin domain expression and aberrant mucin domain glycosylation patterns have been implicated in disease pathologies, especially in tumor progression, where mucins modulate immune responses and also promote proliferation through biomechanical mechanisms.

Mucin domains also exist in proteins outside of the 21 canonical mucins. For example, CD43 on the surface of leukemia cells interacts with the glyco-immune checkpoint receptor Siglec-7 through its N-terminal mucin domain; mucin domain-containing splice variants of CD44 (CD44v) serve as cancer cell markers relative to the ubiquitously expressed standard isoform; CD45 mucin domains act as suppressors of T-cell activation; mucin domain O-glycosylation on PSGL-1 is required for leukocyte-endothelial interactions; and aberrant regulation of mucin domains in podocalyxin and SynCAM1 are implicated in a variety of cancers.

The mechanisms by which mucins exert their functions at these surfaces fall broadly into two categories (FIG. 1a). First, mucins are critical to the initiation and propagation of biophysical signals. For example, their extended and rigid secondary structure enables their use by cells as force-sensitive antennae, as is the case for the mucin CD45 during macrophage pinocytosis². Second, the glycopeptide epitopes presented by mucins act as ligands for various receptors, particularly those involved in cell adhesion and immune modulation³.

Cancers, and especially carcinomas, hijack mucin signaling pathways to protect themselves from both biophysical and immunological insults. It is estimated that just one member of the mucin family, MUC1, is aberrantly expressed in greater than half of carcinomas diagnosed per year in the U.S.⁴, a frequency matched by prototypical oncogenes such as RAS and MYC. In addition, common carcinomas such as breast and ovarian cancer have mucinous forms, wherein tumor cells present as individual colonies suspended in a matrix of secreted mucin and polysaccharides⁵. Decades of functional, genetic, and preclinical data support depletion of cancer-associated mucins as a strategy to reverse tumor aggressiveness in a range of carcinomas⁶.

Mucins have, however, remained canonically undruggable. Therapeutic interventions face the challenge that mucin signaling occurs through the cooperative action of hundreds of arrayed epitopes and a unique, scaffolding secondary structure. There is no catalytic site to inhibit with a small molecule, nor is there a discrete functional extracellular epitope amenable to blocking with an antibody.

### SUMMARY

Provided are mucin-active proteases. In certain embodiments, the mucin-active proteases are stably associated with a targeting moiety. According to some embodiments, the mucin-active protease is stably associated with the targeting moiety via fusion of a protein domain comprising the mucin-active protease and a protein domain comprising the targeting moiety. In other embodiments, the mucin-active protease is stably associated with the targeting moiety via conjugation. Also provided are methods of treating a mucin-associated condition in a subject in need thereof, such methods comprising administering to the subject an effective amount of a mucin-active protease of the present disclosure. Upon administration of the mucin-active protease to the subject, the targeting moiety targets the mucin-active protease to cell surface, extracellular and/or secreted mucins, and the mucin-active protease degrades the mucins.

### BRIEF DESCRIPTION OF THE FIGURES

FIG. 1A-1F: Mucinase treatment reverses mucin-driven cancer progressive pathways in cell lines. 1A: Schematic depicting that mucins influence membrane biophysics and immune surveillance. 1B: Setup for suspension survival assay under anchorage-free conditions using MCF10A cells expressing doxycycline-inducible MUC1 ectodomain treated with or without StcE mucinase. 1C: Viability of MCF10A^{±MUC1} cells±10 nM StcE over 72 h as determined by flow cytometry (n=3 biological replicates). 1D: Setup for NK cell killing assay with mucinase-treated leukemia cell lines. 1E: Surface CD43 levels of K562 cells±50 nM StcE measured by flow cytometry (n=3 biological replicates) MFI= mean fluorescence intensity and AU=arbitrary units. 1F: Normalized NK cell killing of K562 cells under indicated StcE treatment conditions at a 2:1 effector: target ratio (n=3 biological replicates). Data are mean+s.e.m. (c) or mean+s.d. (e-f). P-values were determined using Tukey-corrected two-way ANOVA. * p<0.05, ** p<0.005, *** p<0.0005.

FIG. 2A-2I: Structure-guided engineering of StcE yields mutants of reduced size and activity. 2A: Structure of StcE, as predicted by ColabFold (Methods) 47, with the C domain (purple) and INS domain (blue) highlighted. The Zn²⁺ active site is depicted in orange while mutated residues are shown in teal. 2B: Digestion of IRDye 800CW-labeled purified human mucin C1-INH with 50 nM StcE or StcE mutants, quantified by in-gel fluorescence. 2C: Quantification of B (n=4 independent digestions). 2D: Setup for flow cytometry assays measuring cell surface activity and binding of StcE and StcE mutants. 2E: Representative flow plots showing surface MUC1 levels of Hela cells treated with StcE mutants at indicated concentrations. For flow plots of all other StcE mutants, see FIG. 15d. 2F: EC50 values derived quantifying mean fluorescence intensity from from (e) and FIG. 14d (n=3 biological replicates). For dose response curves see FIG. 15e. 2G: Representative flow plots depicting cell surface binding of StcE variants on HeLa cells measured by anti-His staining. For flow plots of all other StcE mutants, see FIG. 15f. 2H: Kd values derived from quantifying mean fluorescence intensity from (g) and FIG. 15f (n=3 biological replicates). For dose response curves see FIG. 15g. 21: ddStcE^{W36A} cleavage motif as determined by mass spectrometry on recombinant mucins. Data are mean±s.d. P-values were determined using Tukey-corrected one-way ANOVA. * p<0.05, ** p<0.005, *** p<0.0005.

FIG. 3A-3E: An optimized nanobody-mucinase fusion protein selectively cleaves mucins from HER2+ cells. 3A: Schematic depicting reversal of mucin-driven tumor progressive pathways via treatment with a targeted nanobody-mucinase conjugate. 3B: Structure of nanobody-mucinase conjugate, as predicted by ColabFold (Methods)⁴⁷, with engineering strategy shown. The HER2-targeting nanobody is depicted in green, active site is shown in orange, mutated residue (W366A) in teal, and flexible linker in yellow. 3C: Binding curves of aHER2-eStcE on MCF10^{A±MUC1,±HER2} cells quantified by anti-His fluorescence intensity as in FIG. 2d (n=3 biological replicates). For flow plots and Kds see FIG. 7c, f-g. 3D: Representative flow plots depicting surface CD43 levels of mixed K562^{±HER2} cells treated with 1 nM mucinases or conjugate overnight. For representative flow plots with shorter incubation times see FIG. 9e. 3E: CD43 cleavage curves derived from mean fluorescence intensity values from (d) (n=3 biological replicates). Data are mean±s.d.

FIG. 4A-4J: aHER2-eStcE is effective in mixed cell assays and breast cancer mouse models. 4A: Setup for mixed cell suspension survival assay under anchorage-free conditions as in FIG. 1b. 4B: Viability of mixed MCF10^{AMUC1,±HER2} cells±1 nM StcE or aHER2-eStcE over 72 h as determined by flow cytometry (n=3 biological replicates). 4C: Setup for mixed cell NK cell killing as in FIG. 1d. 4D: Normalized NK cell killing of mixed K562^{±HER2} cells treated with conjugate at a 2:1 effector: target ratio as determined by flow cytometry (n=3 biological replicates) Values are reported as fold changes relative to the average of three PBS-treated control replicates (dotted line). 4E: Treatment regimen for Balb/c mice injected intravenously (I.V.) via tail vein with 4T07^{MUC1,HER2} cells. αHER2-eStcE was injected I.V. every other day at 10 mg/kg starting on day 2 (n=7 animals per group). 4F: Plot depicting lung masses of animals described in (e). 4G: Percent area of lung metastases quantified by H&E tissue staining of animals described in (e). Points represent individual scans of lung slices (n=2 per animal). For images, see FIG. 13f-g. 4H: Treatment regimen for Balb/c mice injected with EMT6^{HER2} orthotopically into the mammary fat pad. αHER2-eStcE or αHER2 were injected four times intraperitoneally (I.P.) every other day starting on day 8 (n=9-12 animals per group). The dose was 10 mg/kg for αHER2-eStcE and an equimolar quantity (2.8 nmol) of αHER2. 41: Average growth curves of EMT6^{HER2} tumors for animals described in (h). 4J: Survival curves for animals described in (h-i). Data are mean±s.e.m. (b, f-g, i) or mean±s.d. (d). P-values were determined using Tukey-corrected two-way ANOVA (d, i), Mann-Whitney test (f-g) and Mantel-Cox test (j). * p<0.05, ** p<0.005, *** p<0.0005.

FIG. 5A-5H: StcE treatment of cell lines potentiates NK cell surveillance and small molecule-induced ferroptosis. 5A-5B: Surface CD43 (5A) and Siglec-7 ligand (5B) levels of CCRF-CEM and CCRF-HSB-2 cells±50 nM StcE measured by flow cytometry (n=3 biological replicates). Siglec-7-Fc staining of K562 cells can be found in Wisnovsky et al. (2021)¹¹. MFI= mean fluorescence intensity and AU=arbitrary units. 5C: Normalized NK cell killing of CCRF-CEM and CCRF-HSB-2 cells±10 nM StcE at 5:1 and 2:1 effector: target ratios, respectively, as determined by flow cytometry (n=3 biological replicates). Tx=treatment. 5D: Cytotoxicity of StcE treatment of leukemia cell lines (n=3 biological replicates). A statistically significant baseline toxicity increase was observed. 5E: Screening strategy for bioactive compound library on OVCAR-3^{N} cells±StcE. Superscript N denotes stable expression of nuclear fluorescent protein. 5F: Normalized area-under-the-curve (nAUC) of lethal fraction scores of OVCAR-3^{N} cells treated with 500 nM bioactive compounds+50 nM StcE. Ferroptosis-inducing erastin and erastin² are highlighted in pink. 5G: Visualization of live (red) and dead (green) OVCAR-3^{N} cells±50 nM StcE±500 nM erastin² (Era2) at 72 hours. Scale bar, 30 μm. 5H: Lethal fraction curves of OVCAR-3N cells treated with 500 nM erastin² or RSL3±50 nM StcE±1 μM ferrostatin-1 (Fer-1) for 48 hours (n=5 biological replicates). Data are mean±s.d. P-values were determined using Tukey-corrected two-way ANOVA. * p<0.05, ** p<0.005, *** p<0.0005.

FIG. 6A-6D: StcE cleaves mucins in mouse tissues at a maximum tolerated dose of 0.25 mg/kg and exhibits systemic toxicity at higher doses. 6A: Necropsy analysis post intravenous (I.V.) injection of 15 mg/kg StcE revealed abnormalities in the lung, gastrointestinal tract, and underneath the skull (n=1 animal). 6B: SDS-PAGE of plasma and tissues post intraperitoneal (I.P.) injection of PBS or 0.25 mg/kg of IRdye 800CW-labeled StcE (StcE-800, molecular weight=98 kDa), indicated by the black arrow. 6C: Mucin Western blot on plasma and tissues from (6B). Mucin bands are indicated by black arrows. 6D: Mucin Western blot on plasma and tissues 3 hours post I.P. injection of PBS or 10 mg/kg StcE. Mucin bands are denoted by black arrows.

FIG. 7A-7G: Expression and characterization of engineered nanobody-mucinase conjugates. 7A: Digestion of recombinant MUC16 (rhMUC16) with eStcE alone or nanobody-eStcE conjugates. 7B: rhMUC16 in-gel digest depicting degradation of eStcE-αHER2 conjugate after long-term storage at 4° C. 7C: Representative flow plots showing cell surface binding of nanobody alone and eStcE-αHER2 on MCF10AHER2 cells measured by anti-His staining. 7D: Cell surface binding curves derived from mean fluorescence intensity from (c) (n=3 biological replicates). 7E: Kd values derived from (d). 7F: Representative flow plots showing cell surface binding of αHER2-eStcE on MCF10A±MUC1, ±HER2 cells measured by anti-His staining. For flow plot of αHER2-eStcE on MCF10AHER2 see (c). 7G: Kd values derived from mean fluorescence intensity from (f) and FIG. 3c (n=3 biological replicates). Data are mean±s.d. P-values were determined using Tukey-corrected one-way ANOVA. * p<0.05, ** p<0.005, *** p<0.0005.

FIG. 8A-8F: Assessment of αHER2-eStcE selectivity for mucin substrates in vitro and on cell surfaces. 8A: Digestion of recombinant or purified non-mucins (BSA, fetuin) and mucins (C1-INH, CD43, PODXL, PSGL-1) with StcE, StcE mutants, and αHER2-eStcE. 8B: Setup for terminal amine isotopic labeling of substrates mass spectrometry (TAILS MS) experiment. Mucinase-generated peptides derived from mucin domains were not searched for because of search space complications caused by glycan modifications. 8C-8E: Volcano plots depicting enrichment of peptides following treatment of K562^{HER2} cells with αHER2-eStcE (8C), StcE (8D), or eStcE (8E) relative to vehicle control (n=4 biological replicates). 8F: Annotation of predicted O-glycosites (yellow squares) 10 and known phosphosites (blue circles) 49 in putative mucin domains of enriched proteins from (8C-8E) (from top to bottom: SEQ ID NOs: 16-19).

FIG. 9A-9G: Mixed cell assays to assess targeted de-mucination using generated HER2⁺ cell lines. 9A-9D: Surface HER2 levels of K562^{±HER2} (9A), MCF7^{±HER2} (9B), MCF10A^{±HER2} (9C), and 4T07^{±HER2} (9D) cells measured by flow cytometry. 9E: Representative flow plots depicting surface CD43 levels of mixed K5621^{HER2} cells treated with StcE or conjugate for the indicated times and concentrations. 9F: Representative flow plots depicting surface MUC1 levels of mixed MCF10A^{±MUC1,±HER2} cells treated with 10 nM mucinases or conjugate. MCF10A^{±MUC1,±HER2} cells were pre-labeled with CellTracker Green CMFDA. 9G: MUC1 cleavage curves derived from mean fluorescence intensity from (9F) (n=3 biological replicates). Data are mean±s.d.

FIG. 10A-10C: αHER2-eStcE expands the therapeutic window for selective cleavage of mucins from HER2⁺ cells as compared to αHER2-StcE. 10A: Representative flow plots depicting surface CD43 levels of mixed K562^{±HER2} cells treated with StcE, αHER2-StcE, eStcE, or αHER2-eStcE for 1 hour. 10B: CD43 cleavage curves derived from median fluorescence intensity from (10A) (n=3 biological replicates). 10C: Selective cleavage of mucins on HER2⁺ cells derived from the ratio of CD43 median fluorescence intensity on K562 divided by CD43 median fluorescence intensity of K562^{HER2} from (10A). Data are mean±s.d.

FIG. 11A-11E: The targeted mucin degradation approach is generalizable for cell surface binding targets. 11A: Schematic depicting targeted mucin degradation using a single anti-mouse IgG1-mucinase (αIgG1-eStcE) conjugate and primary mouse IgG1 antibodies against non mucin-associated, mucin, and mucin-associated cell surface epitopes. 11B: Flow cytometry plots showing maximum K562^{HER2} cell surface staining achieved with each primary (1.25-20 μg/mL) and Alexa Fluor 647 anti-mouse IgG1 secondary. To test if anti-mucin antibodies were StcE-sensitive, K562^{HER2} cells were treated with 100 nM StcE for 1 hour. The MUC1 antibody was previously confirmed to be StcE-sensitive in FIG. 9F. 11C: Representative flow plots depicting surface CD43 levels of K562^{HER2} cells treated with StcE, eStcE, and αHER2-eStcE, or indicated primary antibody and αIgG1-eStcE for 4 hours. 11D: CD43 cleavage curves derived from median fluorescence intensity from (11C) (n=3 biological replicates). 11E: EC50 of CD43 cutting derived from curve fitting (11D) were compared to the maximum median fluorescence intensity of primary binding in (11B) (left), target's mucinome enrichment score (Examples Methods) (center) 48, or the concentration of primary used in the cutting experiment (right). The dotted lines represent mean EC50 values for eStcE, StcE, and αHER2-eStcE from (11D). Mucins were excluded from the left plot since their MFI changes during mucin depletion, and HER2 and isotype were excluded from the center plot since they do not have a mucinome score. Data are mean±s.d.

FIG. 12A-12F: αHER2-eStcE is nontoxic to mice at every tested dose and distributes widely across tissues. 12A: SDS-PAGE of plasma from mice post retro-orbital injection of PBS or IRdye 680RD-labeled αHER2-eStcE (αHER2-eStcE-680) at the indicated doses. αHER2-eStcE is indicated by the black arrow. 12B: SDS-PAGE of plasma and tissues post retro-orbital injection of 10 mg/kg αHER2-eStcE-680, indicated by the black arrow. 12C: Necropsy analysis 3 hours post retro-orbital injection of 10 mg/kg αHER2-eStcE-680 revealed no abnormalities. 12D: Mucin Western blot on plasma and tissues 4 hours post retro-orbital injection of 5 mg/kg StcE or conjugate. Mucin bands are denoted by black arrows. 12E: Treatment regimen and protocol for FITC-dextran permeability assay. 12F: Concentration of FITC-dextran in plasma of BALB/c mice treated with PBS or αHER2-eStcE (n=5 animals per group). Data are mean±s.d. P-values were determined using two-tailed unpaired t-test.

FIG. 13A-13I: In the 4T07^{MUC1,HER2} murine model of breast cancer progression, αHER2-eStcE reduces lung metastatic burden and the prosurvival mechanosignaling markers, p-FAK-Y397 and cyclin D1. 13A-13B: 4T07^{MUC1} cells (13A) and OVCAR-3 cells (13B) were treated with 50 nM StcE for 2 hours, washed 1× with 2 mM EDTA followed by 5× with DPBS, then cultured for the indicated times. Cells were then lysed and subjected to Western blotting for MUC1 (13A) and MUC16 (13B). Mucin bands are denoted by black arrows. 13C: Bioluminescent imaging of animals described in FIG. 4E. 13D: Total flux measurements quantified from (13C). 13E: Plot depicting mouse masses of animals described in FIG. 4E. 13F-13G: H&E staining of lungs from PBS-treated (13F) or αHER2-eStcE treated (13G) animals (n=7 animals per group, 2 slides per animal). Percent area of lung metastases is quantified in FIG. 4G. 13H: Quantification of images from FIG. 20 using the IHC profiler plugin in ImageJ. Percent positive corresponds to positive DAB staining in the cytosol. 131: Quantification of images from FIG. 21 using the IHC profiler plugin in ImageJ. Percent positive corresponds to positive DAB staining in the cytosol. 13J: Quantification of images from FIG. 22 using the IHC profiler plugin in ImageJ. Percent positive corresponds to positive DAB staining in the nucleus. 13K: Treatment regimen for BALB/c mice injected intravenously (I.V.) via tail vein with 4T07^{MUC1,HER2} cells. Doxycycline was included in the chow for the duration of the experiment to maintain MUC1 ectodomain expression. αHER2-eStcE at 10 mg/kg or an equimolar quantity of αHER2-eStcEE447D or aGFP-eStcE were injected I.V. every other day starting on day 0 (n=9 animals per group). 13L: Total flux of the indicated days normalized to the total flux on day 0 for each mouse quantified from FIG. 24. Data are mean±s.e.m. P-values were determined using Mann-Whitney test (13D-13E), two-tailed unpaired t-test (13H-13J) , or Tukey-corrected one-way ANOVA (I). * p<0.05, ** p<0.005, *** p<0.0005.

FIG. 14A-14O: In the EMT6^{HER2} murine model of breast cancer progression, αHER2-eStcE reduces mucin levels on EMT6^{HER2} cells but not immune cells and alters the tumor immune microenvironment. 14A: Plot depicting mouse masses of animals described in FIG. 4H. Mouse masses for αHER2 treated mice were not measured. 14B: Treatment regimen for BALB/c mice injected with EMT6^{HER2} orthotopically into the mammary fat pad. αHER2-eStcE, αHER2-eStcEE447D, or aGFP-eStcE were injected four times intraperitoneally (I.P.) every other day starting on day 8 (n=3-6 animals per group). The dose was 10 mg/kg for αHER2-eStcE or an equimolar quantity of αHER2-eStcE^{E447D} or αGFP-eStcE. 14C: Average growth curves of EMT6^{HER2} tumors for animals described in (14B). Mice were euthanized once tumor size reached approximately 1500 mm³ or when mice developed ulcerated tumors. 14D: Short-term treatment regimen for BALB/c mice injected with EMT6^{HER2} orthotopically into the mammary fat pad. αHER2-eStcE or αHER2 were injected twice intraperitoneally (I.P.) every other day starting on day 8 (n=6-9 animals per group). The dose was 10 mg/kg for αHER2-eStcE and an equimolar quantity (2.8 nmol) of αHER2. 14E: Total cell surface mucin staining of CD45⁻/HER2⁺ (EMT6^{HER2} subset) and CD45⁺/HER2⁻ (immune subset) cells isolated from animals described in (13D) using Alexa Fluor 647-labeled StcE^{E447D} (AF647-StcE^{E447D}) 19. Subsets were defined as shown in FIG. 25. 14F: Mean fluorescence intensity (MFI) values derived from (14E) (n=2-5 biological replicates). 14G-141: T-statistical stochastic neighbor embedding (tSNE) plots depicting immune cell subsets of tumor-infiltrating lymphocytes from untreated (14G), αHER2 treated (14H), and αHER2-eStcE treated (141) animals described in (14D). Immune subsets were defined as shown in FIG. 26. 14J: Live single CD45+ cells per gram of tumor. 14K: Percent of tumor-infiltrating Ly6G+ cells as a fraction of total CD45+ cells. 14L: Percent of PD-1+ cells in the Ly6G+ cell population. 14M: Percent of tumor-infiltrating cDCs, cDC Type 1 (cDC1s), and cDC Type 2 (cDC2s) as a fraction of total CD45+ cells. 14N-14O : Percent of PD-L1+ (14N) and GzmB+ (14O ) cells in cDC, cDC1, and cDC2 populations. n=6-9 animals per group. Data are mean±s.e.m (14A-14B) or mean±s.d (14F,¹⁴J-14O ). P-values were determined using Tukey-corrected one-way (14J-14L) and two-way ANOVA (14A, 14C, 14F, 14M^{-14O ) . * p<}0.05, ** p<0.005, *** p<0.0005.

FIG. 15A-15G: Design, expression, and characterization of engineered StcE mutants. 15A: Docking of glycopeptide Ac-P(GalNAc) TLTH-NMe into the structure of StcE determined using AlphaFold⁶⁵. The INS domain (blue) is highlighted. The Zn²⁺ active site is depicted in orange while mutated residues are shown in teal. The glycopeptide backbone is shown in green and the GalNAc sugar is depicted in yellow. 15B: SDS-PAGE of purified StcE and StcE mutants. 15C: Digestion of rhMUC16 with 50 nM StcE or StcE mutants at 37° C. for 1 hour. 15D: Representative flow plots related to FIG. 2E-2F showing surface MUC1 levels of Hela cells treated with StcE variants at indicated concentrations. 15E: MUC1 cleavage curves for StcE and StcE mutants corresponding to mean fluorescence intensity from FIG. 2E-2F and (15D) (n=3 biological replicates). 15F: Representative flow plots related to FIG. 2G-2H depicting cell surface binding of StcE variants on Hela cells measured by anti-His staining (n=3 biological replicates). 15G: Binding curves for StcE and StcE mutants corresponding to normalized mean fluorescence intensity from FIG. 2g-h and (15F) (n=3 biological replicates). Data are mean±s.d.

FIG. 16A-16D: αHER2-eStcE does not need to bind mucins stably in order to deplete cellular mucins. 16A-16B: Representative flow plots showing the change in CD43 cell surface levels (16A) and binding (16B) to K562^{HER2} cells following different incubation times with 100 nM of αHER2, αHER2-eStcE, or StcE. 16C-16D: Time-dependent CD43 cleavage determined via quantification of the normalized median fluorescence intensities (16C) and plot of the median fluorescence intensities depicting αHER2, αHER2-eStcE, or StcE cell surface residency (16D) from (16A-16B).

FIG. 17A-17D: Validation of αIgG1-eStcE. 17A: SDS-PAGE of purified αHER2-eStcE, αHER2-StcE, and αIgG1-eStcE. 17B: Flow plots depicting specific binding of αIgG1-eStcE to mouse IgG1 antibodies. CellTrace Violet stained K562^{HER2} cells were mixed with unstained K562 cells, stained with primary mouse IgG1 anti-HER2 and secondary Alexa Fluor 647-labeled αIgG1-eStcE or no primary and Alexa Fluor 647-labeled αHER2-eStcE. 17C: Cell surface binding curves derived from normalized median fluorescence intensities from (17B) (n=3 biological replicates). 17D: Flow plots of αIgG1-eStcE activity in a mixed cell cutting assay. Mixed K562 cells and K562^{HER2} cells were treated overnight with αHER2-eStcE or 10 μg/mL anti-HER2 mouse IgG1 and αIgG1-eStcE.

FIG. 18A-18B: Gating strategy for mixed NK cell killing assay. 18A-18B: Gating strategy used to define the populations in FIG. 4D using a representative 10 nM αHER2-eStcE treated K562^{±HER2} sample (18A) and a representative 10 nM αHER2-eStcE and NK cell treated K562^{±HER2} sample (18B). K562^{±HER2} were gated from NK cells using FSC-A vs SSC-A given the different sizes of the two cell populations. Values shown on the graph are the percentage of cells from the parent population in each gate from these representative replicates.

FIG. 19A-19C: αHER2-eStcE potentiates macrophage phagocytosis in a mixed cell assay. 19A: Setup for mixed cell macrophage phagocytosis assay using MCF7^{±HER2} cells. 19B: Relative binding index of MCF7^{±HER2} cells treated with the phagocytosis inhibitor cytochalasin D, StcE, or αHER2-eStcE (n=3 biological replicates). 19C: Representative confocal microscopy images used for (19B). Data are mean±s.d. P-values were determined using multiple unpaired t-tests with two-stage Benjamini, Kreiger, and Yekutieli false discovery rate correction. * p<0.05, ** p<0.005, *** p<80 0.0005.

FIG. 20A-20B: In the 4T07^{MUC1,HER2} murine model of breast cancer progression, αHER2-eStcE reduces prosurvival mechanosignaling marker, pAkt. 20A-20B: pAkt immunohistochemistry of lungs from PBS-treated (20A) or αHER2-eStcE treated (20B) animals described in FIG. 4E. Each image represents a unique field-of-view.

FIG. 21A-21B: In the 4T07^{MUC1,HER2} murine model of breast cancer progression, αHER2-eStcE reduces the prosurvival mechanosignaling marker, p-FAK-Y397. 21A-21B: p-FAK-Y397 immunohistochemistry of lungs from PBS-treated (21A) or αHER2-eStcE treated (21B) animals described in FIG. 4E. Each image represents a unique field-of-view.

FIG. 22A-22B: In the 4T07^{MUC1,HER2} murine model of breast cancer progression, αHER2-eStcE reduces the prosurvival mechanosignaling marker, cyclin D1. 22A-22B: Cyclin D1 immunohistochemistry of lungs from PBS-treated (21A) or αHER2-eStcE treated (21B) animals described in FIG. 4E. Each image represents a unique field-of-view.

FIG. 23A-23B: Generation and validation of αGFP-eStcE and αHER2-eStcE^{E447D}. 23A: SDS-PAGE of purified αHER2-eStcE^{E447D}, αGFP-eStcE, and αHER2. The two αHER2-eStcE lanes represent different purification batches. 23B: Flow plots depicting surface CD43 levels of mixed K562^{±HER2} cells treated with StcE, αHER2, αHER2-eStcE, αGFP-eStcE, or αHER2-eStcE^{E447D} overnight.

FIG. 24: Bioluminescent imaging of animals described in FIG. 13K.

FIG. 25: Gating strategy for EMT6^{HER2} and immune cells. Gating strategy for Extended Data FIG. 14E-14F. Fluorescence minus one controls (FMO) were used to define negative staining gates. The values given are the percentage of cells from the parent population in each gate from these representative replicates.

FIG. 26A-26B: Gating strategy for EMT6^{HER2} immune subset profiling. 26A: Gating strategy used to define different immune subsets in Extended Data FIG. 14G-14O from live single cells. Plots are from a representative αHER2-eStcE treated mouse, with the percentages of cells within each gate of the parent population from this representative sample shown. 26B: Defining gates for positive PD-1, PD-L1, and GzmB staining in FIG. 14L, 14N-14O with immune subsets from the same mouse as in (26A). Top plots show gates on stained populations and bottom plots show gates on a fully unstained sample. The values given are the percentage of cells from the parent population in each gate from the representative samples shown.

### DETAILED DESCRIPTION

Before the mucin-active proteases and methods of the present disclosure are described in greater detail, it is to be understood that the mucin-active proteases and methods are not limited to particular embodiments described, as such may, of course, vary. It is also to be understood that the terminology used herein is for the purpose of describing particular embodiments only, and is not intended to be limiting, since the scope of the mucin-active proteases and methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the mucin-active proteases and methods. The upper and lower limits of these smaller ranges may independently be included in the smaller ranges and are also encompassed within the mucin-active proteases and methods, subject to any specifically excluded limit in the stated range. Where the stated range includes one or both of the limits, ranges excluding either or both of those included limits are also included in the mucin-active proteases and methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the mucin-active proteases and methods belong. Although any mucin-active proteases and methods similar or equivalent to those described herein can also be used in the practice or testing of the mucin-active proteases and methods, representative illustrative mucin-active proteases and methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present mucin-active proteases and methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the mucin-active proteases and methods, which are, for clarity, described in the context of separate embodiments, may also be provided in combination in a single embodiment. Conversely, various features of the mucin-active proteases and methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present mucin-active proteases and methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Mucin-Active Proteases

Provided by the present disclosure are mucin-active proteases stably associated with a targeting moiety. By “mucin-active protease” is meant an enzyme that catalyzes the hydrolysis of a peptide bond in a mucin domain of a mucin domain-containing glycoprotein (or “mucin”). Mucins are characterized by the presence of one or more mucin domains, which are enriched in proline, threonine, and serine (PTS) amino acids. The serine and threonine amino acids in these mucin domains (also called “PTS domains”) are heavily modified by glycans pointing out in all directions as bristles, giving them a “bottle-brush” like conformation. Due to the hydroxyl groups of the densely packed saccharide polymers, many mucins have a high capacity to bind water giving them a gel-like consistency. Mucins consist mainly of O-glycans in which large glycan chains are attached via N-acetylgalactosamine (GalNAc), and often have a high sialic acid content which renders mucins negatively charged in water and increases their rigidity. The complexity and size of the various glycan chains and the thereby resulting variety of mucins provides a high degree of resistance against proteases.

Mucins are present in high density on all mucosal surfaces including the gastrointestinal, respiratory, reproductive, hepatic, pancreatic and renal epithelium, where they function as protection and barriers against extraneous agents, various microbial pathogens and cells.

The generic structure of transmembrane, i.e., membrane-bound, mucins encompasses a mucin domain, glycan side chains, a central protein core (also called mucin protein backbone), a transmembrane domain and a cytoplasmic tail. Secreted mucins contain only a mucin domain, glycan side chains, and a mucin protein backbone.

The human mucin family (MUC) encompasses 21 mucins (MUC1-21). MUC2, MUC5AC, MUC5B, MUC6, MUC7, MUC8, MUC9 and MUC19 are secreted mucins that protect the epithelium from inflammation, pH changes, toxins and pathogens, while MUC1, MUC3A/B, MUC4, MUC11, MUC12, MUC13, MUC15, MUC16, MUC17, MUC20, MUC21 and MUC22 are transmembrane mucins that may also function as barriers against toxins and pathogens. Aside from the MUC family, there are other O-glycosylated proteins that are “mucin-type” O-glycoproteins and characterized by a mucin domain. As used herein, the terms “mucin domain-containing glycoprotein” or “mucin” will generally refer to those proteins recognized as mucins (e.g., belonging to a mucin family) as well as those proteins containing a mucin domain or otherwise recognized as “mucin-type” or “mucin-like”.

Mucin domain or mucin-type O-glycoproteins are also present either as secreted or as transmembrane mucins on the surface of nearly every cell in the human body, particularly at outer surfaces that lack an impermeable layer, such as the surfaces of the digestive, genital, and respiratory system tracts. Mucin domain-containing glycoprotein contain Ser/Thr-linked a-GalNAc as the initiating, anchoring O-linked glycan (O-glycan). The O-glycan can terminate with a single GalNAc, like the transferrin receptor, or be elaborated to a few dozen O-glycans, like the LDL-receptor, or many dozens, like PSGL-1.

O-linked glycans influence the secondary, tertiary, and quaternary structure of protein, and maintain protein stability, heat resistance, hydrophilicity, and protease resistance. Furthermore, O-linked glycans are involved in immunologic recognition, nonspecific protein interactions, receptor-mediated signaling, modulation of the activity of enzymes and signaling molecules, protein expression, and protein processing.

According to some embodiments, the mucin-active protease is a mucin-selective protease. By “mucin-selective protease” (which may be used interchangeably herein with the term “mucin-specific protease”) is meant a mucin-active protease that preferentially cleaves mucin domain-containing glycoproteins as compared to non-mucin domain-containing glycoproteins.

In certain embodiments, the mucin-active protease selectively recognizes a joint glyco-peptide epitope (an epitope comprising a combination of a particular amino acid sequence and glycosylation status thereof), such that the activity of the enzyme is gated on the glycosylation status of the protein.

According to some embodiments, the mucin-active protease cleaves at a glycan-peptide cleavage motif comprising: S/T *-X-S/T, S/T *-S/T, X-S/T*, S/T *-X, and/or S/T *-X-X-X-X (where * denotes glycosylation of the S or T residue and X is any amino acid residue).

A mucin-active protease of the present disclosure may cleave one or more of a variety of mucin domain-containing glycoproteins. In certain embodiments, the mucin-active protease cleaves one or any combination of C1 esterase inhibitor (C1-INH), cell adhesion molecule 1 (CADM1), CD43, CD44, CD45, CD68, growth-regulated alpha protein (CXCL1), endomucin (EMCN), growth hormone A1 (GHA1), anaerobic glycerol-3-phosphate dehydrogenase subunit A (GLPA), anaerobic glycerol-3-phosphate dehydrogenase subunit C (GLPC), platelet glycoprotein Ib alpha chain (GP1BA), hepatitis A virus cellular receptor 1 (HAVCR1), heart of glass (HEG), mucosal addressin cell adhesion molecule 1 (MADCAM1), mucin-1 (MUC1), Mucin-2 (MUC2), mucin-3A (MUC3A), mucin-3B (MUC3B), mucin-4 (MUC4), mucin-5AC (MUC5AC), mucin-5B (MUC5B), mucin-6 (MUC6), mucin-7 (MUC7), mucin-8 (MUC8), mucin-9 (MUC9), mucin-10 (MUC10), mucin-11 (MUC11), mucin-12 (MUC12), mucin-13 (MUC13), mucin-14 (MUC14), mucin-15 (MUC15), mucin-16 (MUC16), mucin-17 (MUC17), mucin-18 (MUC18), mucin-19 (MUC19), mucin-20 (MUC20), mucin-21 (MUC21), mucin-22 (MUC22), mucin-like protein 3 (MUCL3), prostate androgen-regulated mucin-like protein 1 (PARM1), paired immunoglobulin-like type 2 receptor alpha (PILRA), podocalyxin (PODXL), proteoglycan 4 (PRG4), P-selectin glycoprotein ligand 1 (PSGL1), and/or T-cell immunoglobulin and mucin domain-containing protein 4 (TIMD4), the amino acid sequences of which are available, e.g., in the UniProt database.

In certain embodiments, the mucin-active protease is a eukaryotic mucin-active protease. In other embodiments, the mucin-active protease is a prokaryotic mucin-active protease.

When the mucin-active protease is a prokaryotic mucin-active protease, in certain embodiments, the mucin-active protease is secreted protease of C1 esterase inhibitor (StcE) from Escherichia coli O157: H7. According to some embodiments, the StcE comprises 70% or greater, 75% or greater, 80% or greater, 85% or greater, 90% or greater, 95% or greater, 96% or greater, 97% or greater, 98% or greater, 99% or greater, or 100% amino acid identity with the amino acid sequence set forth in SEQ ID NO:1, or a functional fragment thereof which retains mucin-active protease activity.

According to some embodiments, the mucin-active protease is modified relative to a parental (e.g., wild-type) mucin-active protease in order to confer upon the mucin-active protease one or more desirable properties. Non-limiting examples of such modifications include those that confer reduced enzymatic activity (e.g., via one or more amino acid substitutions, deletions and/or insertions at or near the active site of the protease), reduced non-specific binding (e.g., via one or more amino acid substitutions, deletions and/or insertions within or near a binding domain of the protease), and/or reduced size relative to a parental (e.g., wild-type) mucin-active protease. Reduced enzymatic activity and/or reduced non-specific binding relative to a parental (e.g., wild-type) mucin-active protease may be desirable, e.g., to ensure that the activity of an otherwise toxic mucin-active protease is sufficiently low, such that hydrolysis only occurs when the mucin-active protease is concentrated at its target via binding of the targeting moiety. As proof of concept and demonstrated in the Experimental section below, the inventors rationally designed a modified StcE (sometimes referred to herein as a “variant” or “mutant” StcE) with reduced enzymatic activity and binding while retaining specificity for mucins. This modified StcE (the amino acid sequence of which is set forth in SEQ ID NO:2) comprises deletion of the INS domain and the C domain, thereby reducing enzymatic activity and decreasing nonspecific cell surface affinity, respectively. This modified StcE further comprises the amino acid substitution ddStcE^{W366A} (“eStcE”) near the active site of StcE for reduced enzymatic activity. Accordingly, in some embodiments, the mucin-active protease is a StcE comprising one or more deletions relative to the amino acid sequence set forth in SEQ ID NO:1. In certain embodiments, the one or more deletions comprises a deletion of all or a portion of the C domain. According to some embodiments, the one or more deletions comprises a deletion of all or a portion of the INS domain. In certain embodiments, the StcE comprises one or more amino acid substitutions deletions and/or insertions at or near the active site of the protease. Non-limiting examples of such amino acid substitutions include a substitution at W366, H367, Y457, or any combination thereof. In certain embodiments, the one or more amino acid substitutions comprise W366A, H367A, or both. According to some embodiments, the StcE comprises a deletion of all or a portion of the C domain, a deletion of all or a portion of the INS domain, and a W366A substitution.

In certain embodiments, the mucin-active protease is Pic, ZmpB, ZmpC, BT4244, AM0627, AM0908, AM1514, SmEnhancin, VIBHAR2194, CpaA, ImpA, or OgpA. As will be appreciated with the benefit of the present disclosure, such mucin-active proteases may be engineered to include one or more modifications that confer reduced enzymatic activity (e.g., via one or more amino acid substitutions, deletions and/or insertions at or near the active site of the protease), reduced non-specific binding (e.g., via one or more amino acid substitutions, deletions and/or insertions within or near a binding domain of the protease), and/or reduced size relative to a parental (e.g., wild-type) Pic, ZmpB, ZmpC, BT4244, AM0627, AM0908, AM1514, SmEnhancin, VIBHAR2194, CpaA, ImpA, or OgpA protease.

The amino acid sequences of non-limiting example mucin-active proteases of the present disclosure are provided in Table 1.

TABLE 1
Mucin-active protease amino acid sequences

| | | SEQ ID |
|---|---|---|
| Description | Amino Acid Sequence | NO: |
| StcE amino | ADNNSAIYFNTSQPINDLQGSLAAEVKFAQSQILPAHPKEGDSQPHLT | 1 |
| acid sequence | SLRKSLLLVRPVKADDKTPVQVEARDDNNKILGTLTLYPPSSLPDTIYH | |
| with signal | LDGVPEGGIDFTPHNGTKKIINTVAEVNKLSDASGSSIHSHLTNNALVEI | |
| peptide | HTANGRWVRDIYLPQGPDLEGKMVRFVSSAGYSSTVFYGDRKVTLSV | |
| (residues 1-35) | GNTLLFKYVNGQWFRSGELENNRITYAQHIWSAELPAHWIVPGLNLVI | |
| removed. | KQGNLSGRLNDIKIGAPGELLLHTIDIGMLTTPRDRFDFAKDKEAHREY | |
| INS domain, | FQTIPVSRMIVNNYAPLHLKEVMLPTGELLTDMDPGNGGWHSGTMRQ | |
| W366 and | RIGKELVSHGIDNANYGLNSTAGLGENSHPYVVAQLAAHNSRGNYAN | |
| C domain | GIQVHGGSGGGGIVTLDSTLGNEFSHEVGHNYGLGHYVDGFKGSVH | |
| indicated, in | RSAENNNSTWGWDGDKKRFIPNFYPSQTNEKSCLNNQCQEPFDGHK | |
| order, by | FGFDAMAGGSPFSAANRFTMYTPNSSAIIQRFFENKAVFDSRSSTGFS | |
| underlining | KWNADTQEMEPYEHTIDRAEQITASVNELSESKMAELMAEYAVVKVH | |
| | MWNGNWTRNIYIPTASADNRGSILTINHEAGYNSYLFINGDEKVVSQG | |
| | YKKSFVSDGQFWKERDVVDTREARKPEQFGVPVTTLVGYYDPEGTL | |
| | SSYIYPAMYGAYGFTYSDDSQNLSDNDCQLQVDTKEGQLRFRLANHR | |
| | ANNTVMNKFHINVPTESQPTQATLVCNNKILDTKSLTPAPEGLTYTVN | |
| | GQALPAKENEGCIVSVNSGKRYCLPVGQRSGYSLPDWIVGQEVYVDS | |
| | GAKAKVLLSDWDNLSYNRIGEFVGNVNPADMKKVKAWNGQYLDFSK | |
| | PRSMRVVYK | |
| ddStcE^{W366A} | ADNNSAIYFNTSQPINDLQGSLAAEVKFAQSQILPAHPKEGDSQPHLT | 2 |
| amino acid | SLRKSLLLVRPVKADDKTPVQVEARDDNNKILGTLTLYPPSSLPDTIYH | |
| sequence | LDGVPEGRSGELENNRITYAQHIWSAELPAHWIVPGLNLVIKQGNLSG | |
| (ΔC, ΔINS, | RLNDIKIGAPGELLLHTIDIGMLTTPRDRFDFAKDKEAHREYFQTIPVSR | |
| and W366A) | MIVNNYAPLHLKEVMLPTGELLTDMDPGNGGAHSGTMRQRIGKELVS | |
| with signal | HGIDNANYGLNSTAGLGENSHPYVVAQLAAHNSRGNYANGIQVHGG | |
| peptide | SGGGGIVTLDSTLGNEFSHEVGHNYGLGHYVDGFKGSVHRSAENNN | |
| (residues 1-35) | STWGWDGDKKRFIPNFYPSQTNEKSCLNNQCQEPFDGHKFGFDAMA | |
| removed. | GGSPFSAANRFTMYTPNSSAIIQRFFENKAVFDSRSSTGFSKWNADT | |
| | QEMEPYEHTIDRAEQITASVNELSESKMAELMAEYAVVKVHMWNGN | |
| | WTRNIYIPTASADNRGSILTINHEAGYNSYLFINGDEKVVSQGYKKSFV | |
| | SDGQFWKERDVVDTREARKPEQFGVPVTTLVGYYDPEGTLSSYIYPA | |
| | MYGAYGFTYSDDSQNLSDNDCQLQVDTKEGQLRFRLANHRANNTVM | |
| | NKFHINVPTESQPTQATLVCNNKILDTKSLTPAPEGLTYTVNGQALPAK | |
| Pic from | MNKVYSLKYCPVTGGLIAVSELARRVIKKTCRRLTHILLAGIPAICLCYS | 3 |
| Escherichia | QISQAGIVRSDIAYQIYRDFAENKGLFVPGANDIPVYDKDGKLVGRLGK | |
| coli 042 | APMADFSSVSSNGVATLVSPQYIVSVKHNGGYRSVSFGNGKNTYSLV | |
| | DRNNHPSIDFHAPRLNKLVTEVIPSAVTSEGTKANAYKYTERYTAFYR | |
| | VGSGTQYTKDKDGNLVKVAGGYAFKTGGTTGVPLISDATIVSNPGQT | |
| | YNPVNGPLPDYGAPGDSGSPLFAYDKQQKKWVIVAVLRAYAGINGAT | |
| | NWWNVIPTDYLNQVMQDDFDAPVDFVSGLGPLNWTYDKTSGTGTLS | |
| | QGSKNWTMHGQKDNDLNAGKNLVFSGQNGAIILKDSVTQGAGYLEF | |
| | KDSYTVSAESGKTWTGAGIITDKGTNVTWKVNGVAGDNLHKLGEGTL | |
| | TINGTGVNPGGLKTGDGIVVLNQQADTAGNIQAFSSVNLASGRPTVVL | |
| | GDARQVNPDNISWGYRGGKLDLNGNAVTFTRLQAADYGAVITNNAQ | |
| | QKSQLLLDLKAQDTNVSEPTIGNISPFGGTGTPGNLYSMILNSQTRFYI | |
| | LKSASYGNTLWGNSLNDPAQWEFVGMDKNKAVQTVKDRILAGRAKQ | |
| | PVIFHGQLTGNMDVAIPQVPGGRKVIFDGSVNLPEGTLSQDSGTLIFQ | |
| | GHPVIHASISGSAPVSLNQKDWENRQFTMKTLSLKDADFHLSRNASL | |
| | NSDIKSDNSHITLGSDRAFVDKNDGTGNYVIPEEGTSVPDTVNDRSQY | |
| | EGNITLNHNSALDIGSRFTGGIDAYDSAVSITSPDVLLTAPGAFAGSSL | |
| | TVHDGGHLTALNGLFSDGHIQAGKNGKITLSGTPVKDTANQYAPAVYL | |
| | TDGYDLTGDNAALEITRGAHASGDIHASAASTVTIGSDTPAELASAETA | |
| | ASAFAGSLLEGYNAAFNGAITGGRADVSMHNALWTLGGDSAIHSLTV | |
| | RNSRISSEGDRTFRTLTVNKLDATGSDFVLRTDLKNADKINVTEKATG | |
| | SDNSLNVSFMNNPAQGQALNIPLVTAPAGTSAEMFKAGTRVTGFSRV | |
| | TPTLHVDTSGGNTKWILDGFKAEADKAAAAKADSFMNAGYKNFMTEV | |
| | NNLNKRMGDLRDTNGDAGAWARIMSGAGSADGGYSDNYTHVQVGF | |
| | DKKHELDGVDLFTGVTMTYTDSSADSHAFSGKTKSVGGGLYASALFE | |
| | SGAYIDLIGKYIHHDNDYTGNFASLGTKHYNTHSWYAGAETGYRYHLT | |
| | EDTFIEPQAELVYGAVSGKTFRWKDGDMDLSMKNRDFSPLVGRTGV | |
| | ELGKTFSGKDWSVTARAGTSWQFDLLNNGETVLRDASGEKRIKGEK | |
| | DSRMLFNVGMNAQIKDNMRFGLEFEKSAFGKYNVDNAVNANFRYMF | |
| ZmpB | MNKRKIAAIILATMITNLSATTIDVLAQELNTKNNSKVEVSHDDESHQAR | 4 |
| | VSKFDLYNSDKLDAYNQEFQVSRSNIKSINNNGGKYNSSTIDKAIDGNL | |
| | ETHWETGKPNDANFTNEVVVTFNEITNIDRIVYSARRDSARGKGFAKE | |
| | FEIYASLKDEGDDFNLVSSGEYTESTRDLVEIKFNPTDFKRLKFKFKKA | |
| | DQNWASAAEFMFYKEDKLNEKFNGLFTDSSMNKVSEEFNTLEKLNAF | |
| | ENELKDHPIYDLYKEGLNNARAILTETSENPTKATLGQITYNLNDDYNN | |
| | QYRMPYKNIKAIKNNGRHYAAQNIEKAIDNDVNTYWETGTLNSSSFNN | |
| | EVEVEFNDLVTLDRIVYGSRQSDLKGFAEEVYIYASRTSKGDTYKLVA | |
| | TGAHEATKGLVEAKFEPTEFKRVKFKFKKSKQNSATLNELMFYKPDEV | |
| | YSSIPKLFTDGTMSELSEEFNSLEKINAFKEKAKNHPLYNDFNETIELAE | |
| | SLISNPRKEDVLELEMRGDSISEAKKRKVWNFQDWQITGLSARAGDKI | |
| | TVYVDVAEGDPTPTLLYKQSLTQHGGATSFQLKPGKNEITIPEINYESN | |
| | GIPKDVIQGGDLFFTNYKSDSQKRAPKVRIEGASKYPVFILGKSDENEV | |
| | MKELEAYVEKIKAEPKTTPNIFAVSSNKSLEFVQATYALDWYKKNNKT | |
| | PKYTAEQWDQYIADAMGFWGFDNSKDVNSDFNFRIMPMVKNLSGGA | |
| | FMNAGNGVIGIRPGNQDAILAANKGWGVAHELGHNFDTGGRTIVEVT | |
| | NNMMPLFFESKYKTKTRITDQNIWENNTYPKVGLDDYSNNELYNKAD | |
| | STHLAQLAPLWQLYLYDNTFYGKFERQFRERDFGNKNREDIYKSWVV | |
| | AASDAMELDLTEFFARHGIRVDDKVKEDLAKYPKPDKKIYYLNDLAMN | |
| | YKGDGFTENAKVSVSTSGSNGNIKLSFSVDDENKDNILGYEIRRDGKY | |
| | VGFTSNDSFVDTKSNLDEDGVYVVTPYDRKLNTLNPIEVNALQPTLSV | |
| | NPVITLALGEEFNEEEYIVAKDIKGNSLSESVKVKSSNVNTSKVGEYEV | |
| | LYSLEDSKGNEYTKTSKVNVVSRKEYMSDLTPKQSSNGWGTVRKDK | |
| | SISGGVIGLTRDGDFVDYNKGLGLHSNAEYVYDLEGKDYDYFESYVG | |
| | VDKAMSSRPASSVIFKVLVDGEEKFNSGVMRSTTPQKYVKVDVKNAK | |
| | ELKLIVNDAGDGDSSDHASFGDAKLATLSSKPIIKGENLAYSMDEKVDL | |
| | MKGITATDIEDGNITSKVQIKSSDFVEGKSGIFTVVYSVTDSDGLTSEC | |
| | SRTIAVTDKETQLSDLNWKSATIGSGSVRKDRAVSGNQIRLLNEDNSV | |
| | ETFAKGIGTHSYSEIVYNSEGYDIFDTWVGIDRHVADKKVSSVKFKVYV | |
| | DGELKAETDVMRIDTPKKRLVVDVRNSKEIKLVVDVADNGNNWDHAD | |
| | WADAKFRNLAEYDASELNKAIEEAKKLDLNNYTEESSEALKNAISKGE | |
| | EALLSKDKETINSALEELNKEMNSLVKVDLNAVINIPDKYLLKSIQNQLN | |
| | KTGDITLGDMYSLTTLTLSGVEDLTGLENAKNLETLNMDYNEVKDLRP | |
| | LSKLKKLNTLNAQEQFIAAGELKPSNGKVIGDSKVYNREGKNVAKTIRV | |
| | VDKNGNTILEQDAKDEFTINTKDLSSGLYGVHVLFEDEGFSGVMFYLF | |
| | NV | |
| ZmpC from | LSAYNSQLSIGVGEHLPEPLKIEGYQYIGYIKTKKQDNTELSRTVDGKY | 5 |
| Streptococcus | SAQRDSQPNSTKTSDVVHSADLEWNQGQGKVSLQGEASGDDGLSE | |
| pneumoniae | KSSIAADNLSSNDSFASQVEQNPDHKGESVVRPTVPEQGNPVSATTV | |
| | QSAEEEVLATTNDRPEYKLPLETKGTQEPGHEGEAAVREDLPVYTKP | |
| | LETKGTQGPGHEGEAAVREEEPAYTEPLATKGTQEPGHEGKATVRE | |
| | ETLEYTEPVATKGTQEPEHEGEAAVEEELPALEVTTRNRTEIQNIPYTT | |
| | EEIQDPTLLKNRRKIERQGQAGTRTIQYEDYIVNGNVVETKEVSRTEVA | |
| | PVNEVVKVGTLVKVKPTVEITNLTKVENKKSITVSYNLIDTTSAYVSAKT | |
| | QVFHGDKLVKEVDIENPAKEQVISGLDYYTPYTVKTHLTYNLGENNEE | |
| | NTETSTQDFQLEYKKIEIKDIDSVELYGKENDRYRRYLSLSEAPTDTAK | |
| | YFVKVKSDRFKEMYLPVKSITENTDGTYKVTVAVDQLVEEGTDGYKD | |
| | DYTFTVAKSKAEQPGVYTSFKQLVTAMQSNLSGVYTLASDMTADEVS | |
| | LGDKQTSYLTGAFTGSLIGSDGTKSYAIYDLKKPLFDTLNGATVRDLDI | |
| | KTVSADSKENVAALAKAANSANINNVAVEGKISGAKSVAGLVASATNT | |
| | VIENSSFTGKLIANHQDSNKNDTGGIVGNITGNSSRVNKVRVDALISTN | |
| | ARNNNQTAGGIVGRLENGALISNSVATGEIRNGQGYSRVGGIVGSTW | |
| | QNGRVNNVVSNVDVGDGYVITGDQYAAADVKNASTSVDNRKADRFA | |
| | TKLSKDQIDAKVADYGITVTLDDTGQDLKRNLREVDYTRLNKAEAERK | |
| | VAYSNIEKLMPFYNKDLVVHYGNKVATTDKLYTTELLDVVPMKDDEVV | |
| | TDINNKKNSINKVMLHFKDNTVEYLDVTFKENFINSQVIEYNVTGKEYIF | |
| | TPEAFVSDYTAITNNVLSDLQNVTLNSEATKKVLGAANDAALDNLYLD | |
| | RQFEEVKANIAEHLRKVLAMDKSINTTGDGVVEYVSEKIKNNKEAFML | |
| | GLTYMNRWYDINYGKMNTKDLSTYKFDFNGNNETSTLDTIVALGNSG | |
| | LDNLRASNTVGLYANKLASVKGEDSVFDFVEAYRKLFLPNKTNNEWF | |
| | KENTKAYIVEMKSDIAEVREKQESPTADRKYSLGVYDRISAPSWGHKS | |
| | MLLPLLTLPEESVYISSNMSTLAFGSYERYRDSVDGVILSGDALRTYVR | |
| | NRVDIAAKRHRDHYDIWYNLLDSASKEKLFRSVIVYDGFNVKDETGRT | |
| | YWARLTDKNIGSIKEFFGPVGKWYEYNSSAGAYANGSLTHFVLDRLL | |
| | DAYGTSVYTHEMVHNSDSAIYFEGNGRREGLGAELYALGLLQSVDSV | |
| | NSHILALNTLYKAEKDDLNRLHTYNPVERFDSDEALQSYMHGSYDVM | |
| | YTLDAMEAKAILAQNNDVKKKWFRKIENYYVRDTRHNKDTHAGNKVR | |
| | PLTDEEVANLTSLNSLIDNDIINRRSYDDSREYKRNGYYTISMFSPVYA | |
| | ALSNSKGAPGDIMFRKIAYELLAEKGYHKGFLPYVSNQYGAEAFASGS | |
| | KTFSSWHGRDVALVTDDLVFKKVFNGEYSSWADFKKAMFKQRIDKQ | |
| | DNLKPITIQYELGNPNSTKEVTITTAAQMQQLINEAAAKDITNIDRATSH | |
| | TPASWVHLLKQKIYNAYLRTTDDFRNSIYK | |
| BT4244 | KDTEKSIINSSFSISEEYLIQNLDKSSTSVQIPINTSMELAQWSVSYEAN | 6 |
| from | WLQCSKQKTAAEGTFLRITVNENTGETKRTANIKVTSTTATYTITVNQY | |
| Bacteroides | AKGEVIVEGDIKVTPTGGKASEHQEGQDIENTYDGKFSTDGAAPFHTP | |
| thetaiotaomicron | WGQSAKFPVTLEYYFKGDTEIDYLIYYTRSGNGNFGKVKVYTTTNPDR | |
| | SDYTLQGEYDFKEQNAPSKVSFSEGIKATGIKFEVLSGLGDFVSCDEM | |
| | EFYKTNTDKTLDKQLLTVFTDITCTEIKNNVTNEQIQALPDYFVRIAEAV | |
| | RDNTYDKWEKEFRIRSYEPYSNIAEWADKLMTKKYSDLDNPTGISVKA | |
| | GDDIIVLVGDTYGQNISMQCIWETGTEYKQTASSGDVYMLNPGVNKLT | |
| | MKGEGQLFVMYNTELTSNTAKPIKIHIPLGSGTVNGFFDLKEHKTDEK | |
| | YAELLKKSTHKYFCIRGEKIMFYFHRNKLLEYVPNNILSAIHLWDNIVG | |
| | WQQELMGIDDVRPSQVNNHLFAISPEGSYMWASDYQIGFVYTYLGNI | |
| | LLEDNVMAAEDNAWGPAHEIGHVHQAAINWASSTESSNNLFSNFIIYK | |
| | LGKYKSRGNGLGSVATARYANGQAWYNMGDATHQNEDTETHMRMN | |
| | WQLWIYYHRCEYKTDFWQTLFKLMREVNMTEGEDPGKKQLEFAKMA | |
| | SKAANQNLTDFFEMWGFFEPVNTTIEQYGTYKYYVSDAMIREAKEYM | |
| | AQFPAPKHAFQYIEDRKKSEFPSNDYRYSAVGDVGYYTQFKENQKIT | |
| | KAITAELAGRKVSIQNGDEAVAFELRENDENGKLLYFSTFTTFEIPSSIL | |
| | MVNAKLYAVQADGKRILL | |
| AM0627 | ANTPEHIGNDLKLFKDSSCTSLKPDVKNTSAFQSDAMKELATKILAGH | 7 |
| from | YKPDYLYAEYRALPSPRQTGKNLRIGDGFSKYDNMTGVYLEKGRHVV | |
| Akkermansia | LVGKTEGQEISLLLPNLMRKPAEGVQPTKDPNGWGLHKKQIPLKEGIN | |
| muciniphila | IIDVETPANAYISYFTEDAGKAPKIPVHFVTGKANGYFDTTRGDTNKDW | |
| | VRLLDQAVSPIMDARGKYIQVAYPVEFLKKFTKDRGTELINAYDKLIGIQ | |
| | YQLMGLDKYGKIPENRVLARVNFNYYMFRDGDGVAYLGNDGTMRMV | |
| | TDPENVLKGDACWGFSHEVGHVMQMRPMTWGGMTEVSNNIFSLQA | |
| | AAKTGNESRLKRQGSYDKARKEIIEGEIAYLQSKDVFNKLVPLWQLHL | |
| | YFTKNGHPDFYPDVMEYLRNNAGNYGGNDTVKYQFEFVKACCDVTK | |
| | TDLTDFFEKWGFFKPGKFHIGDYAQYDFNVTPEMVEETKKWIAGKGY | |
| | PKPETDITELSE | |
| AM0908 | KYPSLDVPEDILLHVKEAASSQSPYSGNHSVKQAVDGSMESANWHVP | 8 |
| from | GVHHVEGEFVFEVPETIHYIIFSGANFNEIAVSAMSGSSWKDLGKFDIG | |
| Akkermansia | GSRMIRFKKPLQKVRKIRLTVDYPEGSSPSFTVREISFYKRVESALNRK | |
| muciniphila | LLKVFKDTSCSSINPRCTLTDLKALPEFLQMIAKKIKSGDYEDKEFRIAS | |
| | YKAYSHPEFAAKVRNINALNKFDNPTGIVAEKGDEILVFVGPTHGEDIG | |
| | LASVSPAGIESSSYPLNEGVNKIRINRSGLLYVMYHTDISPPKKPITVHI | |
| | PVGSGIVNGYFDVTRHTDKDWKRMISNAPHSMFDIVGRNSMMILHTK | |
| | YLKDYSPDSITKSVRVWDESVKAMWKIMGFDKYPQPHNNRQLGVSV | |
| | EGGAHMFATWYYCGYSIGDQGNTLKNEVLAPGVLQGNRLWGIGHEI | |
| | GHCYQHPFNWRSMSESSNNFFAQLILDQVTNAINGNEQASDMENPC | |
| | KYLLSEAVKGMPFHDLNGWAKWGFAQYSFYLYFHKLGINPEFYPRLF | |
| | ESLRRKPLSRQAYEVSEAHLALYERICNISRTDFTDDFEIFNWFVPIDR | |
| | KGHQYGDYSFKMTEEMARASKARIAAKRYPKPKFRIAFLHQHGKTVN | |
| | LWGQNLHGSQLNGYWTKYKQNAKLSPSVSASKKDNMIIVRNGENAA | |
| | AFCVVTNGKVVGYYDRQKFDVSGVEWNDTSKVYAIPIQTAEPYKLIYA | |
| | AGRS | |
| AM1514 | QDNLAKRKAAQELTAERKLKEKKAREAAEKQRIKREREIREKKEKERL | 9 |
| from | AAQKAYEEAQEEKARQAAEAARKLQEQAEREEREKRRREELERRER | |
| Akkermansia | EEEARRQEEDTPVEEEPEPEGRFPQPVKNRMPELSVYSIPCRDDIQT | |
| muciniphila | EKDKLLETWSWDKAEKMEGMEEFPTGSSPWKKGKDAGRMQALLEK | |
| | CREWKDAKLASLKACPAAKDFPGVPENGAQTVRRTVEIDSNIGGWHS | |
| | TGLYAPPGAEISCSLSGAPKDGSISVRIGCHTDSLHKLDEWKRVPEIT | |
| | MQVPAGRGRVKMVNPMGGLVYVNVGQRPRRGKVFKVQISGAVPSP | |
| | LFVMGKTTPEQWAEQLENTKAPWGEIRMPRLIVTMPVEQLKQCPDVQ | |
| | KTAEFLQKNMALQDWIMGWDTKPDRLHHPMRFVVDRQISAGAGHSG | |
| | YPAMATKDWTNSIATGSIIHSGSWGLWHELGHNHQSPPFTMEGQTEV | |
| | SVNIFSMVCEVMGTGKDFESCWGGGMGPYGMSAEMKKYFSGTQTY | |
| | NEAPNKVQLFFWVELMYYLGFDAFRQVALQFHDKPYDNGELSDEKK | |
| | WEWVMNAFSKVTGKNMGPFFKIWRTPVSERATGRMKDLPAWLPSK | |
| | DYPACYTAEE | |
| SmEnhancin | MPTITKTQSLYTLTRPVWLDAAGMSKGIDHDRQHLGIILAAGQVLRVR | 10 |
| from | QTNAAFTGKLTLRLLNDDSKTEAEFSVGASWVEAQVNAVSVPFIDTPY | |
| Serratia | GQGEPALEFEYPDTAKALPVYRRGENEAAFFARWDDRDAEFALVDA | |
| marcescens | EYAVLLVPKISKPALKSLGEAKNIDGLIAYYDRIFTFYNALTGVSFEAEH | |
| | ESDRNIRNRYFMKADKHGAGAAYYGGRWTAETSSSVSSFWLTPKDS | |
| | NWGSLHEIAHGYQGHFMGDKYFSTGEMWNNIYAACYQDVMLGERK | |
| | YQEGWLYNYGNAAGVEKKIVDLIAQGTPLNQWDLRSKLFFAMLMVDK | |
| | AGKNAFTHFNQQYRQSCNTPGFVPSEHALLDLLSASFAAAGEQIDVT | |
| | PFVELTGGVITQTQRDLNAFSHAKAVYPLYQLVDAQSLPAVLAQLKLD | |
| | SSLRLVDSSQLKASGLKGDVALSLDIDDFAQIYGEDLVLMEGARYAHK | |
| | VRIDAPTLALNALPIGVYTLRLPTGKDRKYRPTAGYLTVKQGKSAVTLR | |
| | FERKAASPIVSQEINLLGLSDAVFATVVVDRAKGAVVVDVTSTTPHAYF | |
| | PGETYARVTLRDRQGAVRFSKEVQGTQATLSHDELPFAEGDDIEIYHV | |
| | EPSRVRLNPAAAGVIDNKNKTNVLRIETSGLKNPALSGDPEQALLMRL | |
| | EQAAQRLRRLLPAYYAPSSSFKDDIYLAIGAFAEPQREPLLATYRDCLP | |
| | ADNTPPGANVGNAFTVAGKGIGDRQFLTCAVDLVKKTVTVTLAAGIAH | |
| | HYFADSYAFLSVTDADGNTLLSYDVIGSQNQQAKTWVLPLSGYGGEVI | |
| | HLRHEEPDNRLTIVNEMQHVRLAEKGKQQAYCITPVGLKRIKD | |
| VIBHAR2194 | QVIQFNNETPVNNLQGDFEAMVIYAQNITVPHYNDKGDPRPHLTALRD | 11 |
| from | TLLLVKPVEPLNVSSQVKVIARDSQGNQLGALSLNTPEQLPNHDGPNL | |
| Vibrio | DIMYAKDMWSVTLPATWIQPGLTLELLNGSTSGLLSDLDIGAPNELLIN | |
| harveyi | TMDLGMLTNPRGRFYFADNPELHKDYFQKIPVSKLIVNPYETVKLDEV | |
| | MLPDGDLLTELDPSTGTWHQGDMRADTTKILMSHGINLANYGINSSTS | |
| | VSERAHPYTANQITAIAAVGRYQNGVVAHGGSGGNGMVTIDSSVGNE | |
| | WSHEVGHNFGLGHWPGGTDGTTHRPSTDINSAWGWDLFQKRFIANF | |
| | MWNKRNGEDQVCCSDGIGIPAFEGYKFNRDAMGGGEPTSPISKYTLH | |
| | TPYVLEKIQTFMEGKATFDESSPTGFTKWNDETKAMQAFQQPAILLSK | |
| | SITSQSQLNTIKDDIDGSVLLGYINDFDITKVETGDGRWIRDIYLPSAEN | |
| | VAVGKAVNIARYSGYGVTAHVNGQLVNLNRGDSKFYISDGKAWLETT | |
| | EDQVAEGKPTRIPTDYGVPVTTLVGYYDPQQQLDSYIFPALHGAYGFV | |
| | YQPTPVDRLDTTGCYVKVYNGQDHQTDNYQLVGFRFDDNVMNKFHI | |
| | NLKQTDDPTRAEVVCDNAVLSSLDIEKPKQALEVSIVQSDSLKPSENN | |
| | KPVAHAGEDQSVLSGATITLSADKSTDADGDKLTYVWEQISGLPASIQ | |
| | ASNAMTTNVVLAESTQEQSYVFSVLVSDGKSSSSDMVTIIAQPQPTQN | |
| | HAPEVSLPSSIDAKPGEVIEITATASDPDGDVLSFKWSTSGLAYQTMS | |
| | GGTIQLSVPDVTVDSQFLLSVVVSDPKGESASANTTLNVKASGNTCSV | |
| | SDPNATNYDAWSASKSYSGGDLVSYKQLVWKAKHWSQNNQPDSSD | |
| | AWELLSDVVLPWSSQAAYSGGAQVTYNGVKFEAKWWTRGEQPNVS | |
| | SVWINKGAACQ | |
| CpaA | MNFKLKTSLIIGAIVASSLVYAATVLSPNQNNNSGSIPSGYSDLEFNLAN | 12 |
| | GNWVKNLTLPTSANNLDKITIRSSAAYSSYLDTSNTNIPLEVLKINSGDV | |
| | YQFIFNSSQNKWIAQLATVSPTNGATYEVVPLTTASMQKVLIQNEKWA | |
| | QTIALPSDVRDGTTVQVVSTASASSDIDKTNLLFPSSFILKNGSEYWFK | |
| | YYSALGKWVPEYIKPQKLNVQQIGTSLATVNSPLTEVSFGDGNWVSN | |
| | FTLPTTASDRDKIIIKSTATWSAKINNTNINSQATLTLKTGDQYEFMYVS | |
| | DKGYWQLISSPTKVIDSAATIPATLPNMTQPTLKVKLSTSNWQPTLQLP | |
| | AKAQVGDKVVIVSNASADTYINAANGLSTAIKNGENRRFIYTAQGWTV | |
| | DSYTIDMLLVSSPEVNSILGESAAKLRMIEGVNLTNLTAENSNARFYLR | |
| | NVGYLTYKIPAATLKEAISTGRDDTTVQNERKRVLADGVYYQGNEPGD | |
| | GGCGWAWINASAYNMIGANDIAGCSFAAMRHEVGHNLGLYHNGSTNI | |
| | GSGFAHPLGSTAMGGNNINFYSSPYLYNPKYGVRLGVEGKIDAVSVIN | |
| | LNAQKISLYN | |
| IMPa | MSLSTTAFPSLQGENMSRSPIPRHRALLAGFCLAGALSAQAATQEEIL | 13 |
| | DAALVSGDSSQLTDSHLVALRLQQQVERIRQTRTQLLDGLYQNLSQA | |
| | YDPGAASMWVLPANPDNTLPFLIGDKGRVLASLSLEAGGRGLAYGTN | |
| | VLTQLSGTNAAHAPLLKRAVQWLVNGDPGAATAKDFKVSVVGVDKTA | |
| | ALNGLKSAGLQPADAACNALTDASCASTSKLLVLGNGASAASLSATVR | |
| | ARLQAGLPILFVHTNGWNQSSTGQQILAGLGLQEGPYGGNYWDKDR | |
| | VPSSRTRTRSVELGGAYGQDPALVQQIVDGSWRTDYDWSKCTSYVG | |
| | RTTCDDVPGLSDFSKRVDVLKGALDAYNQKAQNLFALPGTTSLRLWL | |
| | LWADAVRQNIRYPMDKAADTARFQETFVADAIVGYVREAGAAQKELG | |
| | SYAGQRQQSMPVSGSEETLTLTLPSAQGFTAIGRMAAPGKRLSIRIED | |
| | AGQASLAVGLNTQRIGSTRLWNTRQYDRPRFLKSPDIKLQANQSVAL | |
| | VSPYGGLLQLVYSGATPGQTVTVKVTGAASQPFLDIQPGEDSSQAIAD | |
| | FIQALDADKADWLEIRSGSVEVHAKVEKVRGSIDKDYGGDVQRFIREL | |
| | NEVFIDDAYTLAGFAIPNQAKTPAIQQECAARGWDCDSETLHKLPGTQ | |
| | HINVDQYAQCGGGCSGNPYDQTWGLNPRGWGESHELGHNLQVNRL | |
| | KVYGGRSGEISNQIFPLHKDWRVLREFGQNLDDTRVNYRNAYNLIVA | |
| | GRAEADPLAGVYKRLWEDPGTYALNGERMAFYTQWVHYWADLKND | |
| | PLQGWDIWTLLYLHQRQVDKSDWDANKAALGYGTYAQRPGNSGDA | |
| | SSTDGNDNLLLGLSWLTQRDQRPTFALWGIRTSAAAQAQVAAYGFAE | |
| | QPAFFYANNRTNEYSTVKLLDMSQGSPAWPFP | |
| OgpA | MLKRLLSAFFSLFFLGAASGTSFAEVTVPDALKDRIALKKTARQLNIVY | 14 |
| | FLGSDTEPVPDYERRLSELLLYLQQFYGKEMQRHGYGARSFGLDIKS | |
| | PGRVNIIEYKAKNPAAHYPYENGGGWKAAQELDEFFKAHPDRKKSQH | |
| | TLIIMPTWNDEKNGPDNPGGVPFYGMGRNCFALDYPAFDIKHLGQKT | |
| | REGRLLTKWYGGMAHELGHGLNLPHNHQTASDGKKYGTALMGSGN | |
| | YTFGTSPTFLTPASCALLDACEVFSVTPSQQFYEGKPEVEVGDVAISF | |
| | KGDQILVSGNYKSPQTVKALNVYIQDPPYAVNQDYDAVSFSRRLGKK | |
| | SGKFSMKIDKKELEGLNNNEFRISLMFILANGLHMQKHFTFHWDALQD | |
| | YRDGSKS | |

According to some embodiments, provided is a mucin-active protease comprising an amino acid sequence comprising 70% or greater, 75% or greater, 80% or greater, 85% or greater, 90% or greater, 91% or greater, 92% or greater, 93% or greater, 94% or greater, 95% or greater, 96% or greater, 97% or greater, 98% or greater, 99% or greater, or 100% amino acid identity with the amino acid sequence set forth in any one of SEQ ID NOs: 1-14, or a functional fragment thereof which retains mucin-active protease activity. In certain embodiments, provided is a “modified”, “variant” or “mutant” version of any of the mucin-active proteases in Table 1, where the mucin-active protease comprises one or more conservative amino acid substitutions relative to a mucin-active protease amino acid sequence set forth in Table 1. A “conservative substitution” is one in which an amino acid is substituted for another amino acid that has similar properties, such that one skilled in the art of peptide chemistry would expect the secondary structure and hydropathic nature of the polypeptide to be substantially unchanged. Modifications may be made in the structure of the polynucleotides and polypeptides contemplated in particular embodiments, polypeptides include polypeptides having at least about and still obtain a functional molecule that encodes a variant or derivative polypeptide with desirable characteristics. When it is desired to alter the amino acid sequence of a polypeptide to create an equivalent, or even an improved, variant polypeptide, one skilled in the art, for example, can change one or more of the codons of the encoding DNA sequence.

As summarized above, a mucin-active protease of the present disclosure is stably associated with a targeting moiety. According to some embodiments, the targeting moiety targets the mucin-active protease to cell surface, extracellular and/or secreted mucins, and the mucin-active protease degrades the mucins. For example, in certain embodiments, the targeting moiety binds to a cell surface molecule of a target cell, where the target cell comprises cell surface mucin domain-containing glycoproteins, and where it is desirable to cleave the cell surface mucin domain-containing glycoproteins using the mucin-active protease, e.g., for therapeutic purposes. In other words, the targeting moiety provides for degradation of cell surface, extracellular and/or secreted mucin domain-containing glycoproteins by the mucin-active protease in a targeted manner in vivo to treat a mucin-associated condition. According to some embodiments, the targeting moiety binds to an extracellular and/or secreted molecule (e.g., an extracellular and/or secreted mucin domain-containing glycoprotein, or an extracellular and/or secreted molecule which colocalizes with extracellular and/or secreted mucin domain-containing glycoproteins), and where it is desirable to cleave the extracellular and/or secreted mucin domain-containing glycoproteins using the mucin-active protease, e.g., for therapeutic purposes. That is, the targeting moiety provides for degradation of extracellular and/or secreted mucin domain-containing glycoproteins by the mucin-active protease in a targeted manner in vivo to treat a mucin-associated condition.

The targeting moiety may vary and may be selected based, e.g., on the nature of the molecule to be targeted, e.g., cell surface molecule on the target cell, or an extracellular or secreted molecule. Non-limiting examples of a targeting moiety that may be employed include a polypeptide, an antibody, a ligand, an aptamer, a nanoparticle, and a small molecule.

In certain embodiments, the targeting moiety specifically binds the target molecule, e.g., a cell surface molecule of the target cell, or an extracellular or secreted target molecule. As used herein, a first molecule “specifically binds” or “preferentially binds” to a target if it binds with greater affinity, avidity, more readily, and/or with greater duration than it binds to other substances, e.g., in a sample. In certain embodiments, the targeting moiety “specifically binds” the target molecule if it binds to or associates with the target molecule with an affinity or Ka (that is, an association rate constant of a particular binding interaction with units of 1/M) of, for example, greater than or equal to about 10⁴ M⁻¹. Alternatively, affinity may be defined as an equilibrium dissociation constant (KD) of a particular binding interaction with units of M (e.g., 10⁻² M to 10⁻¹³ M, or less). In certain aspects, specific binding means the targeting moiety binds to the target molecule with a KD of less than or equal to about 10⁻⁵ M, less than or equal to about 10⁻⁶ M, less than or equal to about 10⁻⁷ M, less than or equal to about 10⁻⁸ M, or less than or equal to about 10⁻⁹ M, 10⁻¹⁰ M, 10⁻¹¹ M, or 10⁻¹² M or less. The binding affinity of the targeting moiety for the target molecule can be readily determined using conventional techniques, e.g., by competitive ELISA (enzyme-linked immunosorbent assay), equilibrium dialysis, by using surface plasmon resonance (SPR) technology (e.g., the BIAcore 2000 or BIAcore T200 instrument, using general procedures outlined by the manufacturer); by radioimmunoassay; or the like. According to some embodiments, the targeting moiety is an antibody. By “antibody” is meant an antibody or immunoglobulin of any isotype (e.g., IgG (e.g., IgG1, IgG2, IgG3, or IgG4), IgE, IgD, IgA, IgM, etc.), whole antibodies (e.g., antibodies composed of a tetramer which in turn is composed of two dimers of a heavy and light chain polypeptide); single chain antibodies (e.g., scFv); fragments of antibodies (e.g., fragments of whole or single chain antibodies) which retain specific binding to the target molecule (e.g., a cell surface molecule of a target cell), including, but not limited to single chain Fv (scFv), Fab, (Fab′)₂, (scFv′)₂, and diabodies; chimeric antibodies; monoclonal antibodies, human antibodies, humanized antibodies (e.g., humanized whole antibodies, humanized half antibodies, or humanized antibody fragments, e.g., humanized scFv); and fusion proteins comprising an antigen-binding portion of an antibody and a non-antibody protein. In certain embodiments, the antibody is selected from an IgG, single chain Fv (scFv), Fab, (Fab)₂, (scFv′) 2, or a single variable domain located on a heavy chain (VHH). According to some embodiments, the antibody is a VHH (sometimes referred to herein and elsewhere as a “nanobody”). The antibody may be detectably labeled, e.g., with an in vivo imaging agent, a radioisotope, an enzyme which generates a detectable product, a fluorescent protein, and the like.

An antibody included as the targeting moiety will vary based on the cell to be targeted. In some embodiments, the antibody specifically binds to an antigen on the surface of a target cell. Target cells of interest include, but are not limited to, cells that are relevant to a particular disease or condition, e.g., a mucin-associated condition. According to some embodiments, the target cell is selected from a cancer cell, an immune cell, and an endothelial cell. As such, in some embodiments, the target cells are cancer cells. By “cancer cell” is meant a cell exhibiting a neoplastic cellular phenotype, which may be characterized by one or more of, for example, abnormal cell growth, abnormal cellular proliferation, loss of density dependent growth inhibition, anchorage-independent growth potential, ability to promote tumor growth and/or development in an immunocompromised non-human animal model, and/or any appropriate indicator of cellular transformation. “Cancer cell” may be used interchangeably herein with “tumor cell”, “malignant cell” or “cancerous cell”, and encompasses cancer cells of a solid tumor, a semi-solid tumor, a primary tumor, a metastatic tumor, and the like. In certain embodiments, the cancer cell is a carcinoma cell.

According to some embodiments, when the target cell is a cancer cell, the targeting moiety specifically binds to a tumor antigen on the surface of the cancer cell. Non-limiting examples of tumor antigens to which the targeting moiety may specifically bind include 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD22, CD25, CD27L, CD30, CD33, CD37, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), EpCAM, ephrin A4 (EFNA4), epidermal growth factor receptor (EGFR), EGFRvlll, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GLUT3, glycoprotein non-metastatic B (GPNMB), guanylate cyclase 2 C (GUCY2C), HCAM, human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), sodium-dependent phosphate transport protein 2B (NaPi2b), Nectin-4, NMB, NOTC_{H}3, p-cadherin (p-CAD), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM⁻¹), trophoblast cell-surface antigen (TROP-2), and VEGF-A.

Non-limiting examples of antibodies that specifically bind to tumor antigens which may be employed as a targeting moiety include Adecatumumab, Ascrinvacumab, Cixutumumab, Conatumumab, Daratumumab, Drozitumab, Duligotumab, Durvalumab, Dusigitumab, Enfortumab, Enoticumab, Figitumumab, Ganitumab, Glembatumumab, Intetumumab, Ipilimumab, Iratumumab, Icrucumab, Lexatumumab, Lucatumumab, Mapatumumab, Narnatumab, Necitumumab, Nesvacumab, Ofatumumab, Olaratumab, Panitumumab, Patritumab, Pritumumab, Radretumab, Ramucirumab, Rilotumumab, Robatumumab, Seribantumab, Tarextumab, Teprotumumab, Tovetumab, Vantictumab, Vesencumab, Votumumab, Zalutumumab, Flanvotumab, Altumomab, Anatumomab, Arcitumomab, Bectumomab, Blinatumomab, Detumomab, Ibritumomab, Minretumomab, Mitumomab, Moxetumomab, Naptumomab, Nofetumomab, Pemtumomab, Pintumomab, Racotumomab, Satumomab, Solitomab, Taplitumomab, Tenatumomab, Tositumomab, Tremelimumab, Abagovomab, Igovomab, Oregovomab, Capromab, Edrecolomab, Nacolomab, Amatuximab, Bavituximab, Brentuximab, Cetuximab, Derlotuximab, Dinutuximab, Ensituximab, Futuximab, Girentuximab, Indatuximab, Isatuximab, Margetuximab, Rituximab, Siltuximab, Ublituximab, Ecromeximab, Abituzumab, Alemtuzumab, Bevacizumab, Bivatuzumab, Brontictuzumab, Cantuzumab, Cantuzumab, Citatuzumab, Clivatuzumab, Dacetuzumab, Demcizumab, Dalotuzumab, Denintuzumab, Elotuzumab, Emactuzumab, Emibetuzumab, Enoblituzumab, Etaracizumab, Farletuzumab, Ficlatuzumab, Gemtuzumab, Imgatuzumab, Inotuzumab, Labetuzumab, Lifastuzumab, Lintuzumab, Lorvotuzumab, Lumretuzumab, Matuzumab, Milatuzumab, Nimotuzumab, Obinutuzumab, Ocaratuzumab, Otlertuzumab, Onartuzumab, Oportuzumab, Parsatuzumab, Pertuzumab, Pinatuzumab, Polatuzumab, Sibrotuzumab, Simtuzumab, Tacatuzumab, Tigatuzumab, Trastuzumab, Tucotuzumab, Vandortuzumab, Vanucizumab, Veltuzumab, Vorsetuzumab, Sofituzumab, Catumaxomab, Ertumaxomab, Depatuxizumab, Ontuxizumab, Blontuvetmab, Tamtuvetmab, or a tumor antigen-binding variant thereof. As used herein, “variant” is meant the antibody specifically binds to the particular antigen (e.g., HER2 for trastuzumab) but has fewer or more amino acids than the parental antibody (e.g., is a fragment (e.g., scFv) of the parental antibody), has one or more amino acid substitutions relative to the parental antibody, or a combination thereof.

In certain embodiments, the targeting moiety is an antibody approved by the United States Food and Drug Administration and/or the European Medicines Agency (EMA) for use as a therapeutic antibody (e.g., for targeting certain disease-associated cells in a patient, etc.), or a fragment thereof (e.g., a single-chain version of such an antibody, such as an scFv version of the antibody) that retains the ability to specifically bind the target antigen.

When an antibody is employed as the targeting moiety, the mucin-active protease may be stably associated with (e.g., conjugated to, fused to, or the like) any convenient portion of the antibody. In certain embodiments, the mucin-active protease is stably associated with a light chain of the antibody, e.g., a kappa (κ) light chain or fragment thereof or a lambda (λ) light chain or fragment thereof. According to some embodiments, the antibody light chain or fragment thereof includes a light chain variable region (V_{L}). Such an antibody light chain or fragment thereof may further include an antibody light chain constant region (C_{L}) or fragment thereof. In certain embodiments, the antibody light chain or fragment thereof is a full-length antibody light chain—that is, an antibody light chain that includes a V_{L} and a C_{L}. In certain embodiments, the mucin-active protease is stably associated with a V_{L} (if present) or a CL (if present), e.g., at or near the N-terminus of a V_{L} or at or near the C-terminus of a C_{L}.

When an antibody is employed as the targeting moiety, the mucin-active protease may be stably associated with a heavy chain or fragment thereof of the antibody. In certain embodiments, the antibody heavy chain or fragment thereof includes a y, a, 0, 8, or u antibody heavy chain or fragment thereof. According to some embodiments, the antibody heavy chain or fragment thereof is an IgG heavy chain or fragment thereof, e.g., a human IgG1 heavy chain or fragment thereof. In certain embodiments, the antibody heavy chain or fragment thereof comprises a heavy chain variable region (V_{H}). Such an antibody heavy chain or fragment thereof may further include a heavy chain constant region or fragment thereof. For example, when the antibody includes a heavy chain constant region or fragment thereof, the antibody heavy chain constant region or fragment thereof may include one or more of a C_{H}1 domain, C_{H}2 domain, and/or C_{H}3 domain. According to some embodiments, the antibody heavy chain is a full-length antibody heavy chain—that is, an antibody heavy chain that includes a VH, a C_{H}1 domain, a C_{H}2 domain, and a C_{H}3 domain. In certain embodiments, the mucin-active protease is stably associated with an Fc region of the antibody. According to some embodiments, the mucin-active protease is stably associated with the antibody at or near the N-terminus of a VH or at or near the C-terminus of a C_{H}3 domain.

According to certain embodiments, the targeting moiety is a ligand. As used herein, a “ligand” is a substance that forms a complex with a biomolecule in nature to serve a biological purpose. The ligand may be a substance selected from a circulating factor, a secreted factor, a cytokine, a growth factor, a hormone, a peptide, a polypeptide, a small molecule, and a nucleic acid, that forms a complex with the target molecule, e.g., a cell surface molecule on the surface of a target cell. In certain aspects, when the targeting moiety is a ligand, the ligand is modified in such a way that complex formation with the target molecule occurs, but the normal biological result of such complex formation does not occur. In certain embodiments, the ligand is the ligand of a cell surface receptor present on a target cell. Cell surface receptors of interest include, but are not limited to, receptor tyrosine kinases (RTKs), non-receptor tyrosine kinases (non-RTKs), growth factor receptors, etc. When the mucin-active protease is stably associated with a ligand as the targeting moiety, the mucin-active protease may be stably associated with any suitable region of the ligand, e.g., a region of attachment that does not interfere or substantially interfere with the ability of the ligand to bind (e.g., specifically bind) the target molecule.

In certain embodiments, the targeting moiety is an aptamer. By “aptamer” is meant a nucleic acid (e.g., an oligonucleotide) that has a specific binding affinity for the target molecule. Aptamers exhibit certain desirable properties for targeted delivery of the mucin-active protease, such as ease of selection and synthesis, high binding affinity and specificity, low immunogenicity, and versatile synthetic accessibility. Aptamers that bind to cell surface molecules are known and include, e.g., TTA1 (a tumor targeting aptamer to the extracellular matrix protein tenascin-C). Aptamers that find use in the context of the present disclosure include those described in Zhu et al. (2015) ChemMedChem 10 (1): 39-45; Sun et al. (2014) Mol. Ther. Nucleic Acids 3: e¹⁸²; and Zhang et al. (2011) Curr. Med. Chem. 18 (27): 4185-4194.

According to some embodiments, the targeting moiety is a nanoparticle. As used herein, a “nanoparticle” is a particle having at least one dimension in the range of from 1 nm to 1000 nm, from 20 nm to 750 nm, from 50 nm to 500 nm, including 100 nm to 300 nm, e.g., 120-200 nm. The nanoparticle may have any suitable shape, including but not limited to spherical, spheroid, rod-shaped, disk-shaped, pyramid-shaped, cube-shaped, cylinder-shaped, nanohelical-shaped, nanospring-shaped, nanoring-shaped, arrow-shaped, teardrop-shaped, tetrapod-shaped, prism-shaped, or any other suitable geometric or non-geometric shape. In certain aspects, the nanoparticle includes on its surface one or more of the other targeting moieties described herein, e.g., antibodies, ligands, aptamers, small molecules, etc. Nanoparticles that find use in the context of the present disclosure include those described in Wang et al. (2010) Pharmacol. Res. 62 (2): 90-99; Rao et al. (2015) ACS Nano 9 (6): 5725-5740; and Byrne et al. (2008) Adv. Drug Deliv. Rev. 60 (15): 1615-1626.

In some embodiments, the targeting moiety is a small molecule. By “small molecule” is meant a compound having a molecular weight of 1000 atomic mass units (amu) or less. In some embodiments, the small molecule is 750 amu or less, 500 amu or less, 400 amu or less, 300 amu or less, or 200 amu or less. In certain aspects, the small molecule is not made of repeating molecular units such as are present in a polymer. In certain aspects, the target molecule is a cell surface receptor for which the ligand is a small molecule, and the targeting moiety is the small molecule ligand (or a derivative thereof) of the receptor. Small molecules that find use as targeting moieties are known. As just one example, folic acid (FA) derivatives have been shown to effectively target certain types of cancer cells by binding to the folate receptor, which is overexpressed, e.g., in many epithelial tumors. See, e.g., Vergote et al. (2015) Ther. Adv. Med. Oncol. 7 (4): 206-218. In another example, the small molecule sigma-2 has proven to be effective in targeting cancer cells. See, e.g., Hashim et al. (2014) Molecular Oncology 8 (5): 956-967. Sigma-2 is the small molecule ligand for sigma-2 receptors, which are overexpressed in many proliferating tumor cells including pancreatic cancer cells. In certain embodiments, a small molecule is employed as the targeting moiety, and it has been demonstrated in the context of a small molecule drug conjugate (SMDC) that the small molecule is effective at targeting a drug to a target cell of interest by binding to a cell surface molecule on the target cell.

In certain embodiments, on-target enzymatic activity is achieved by the use of a low affinity mucin-active protease stably associated with a high affinity targeting moiety. For example, according to some embodiments, the mucin-active protease is mutated such that its substrate affinity (measured, e.g., by effective Kd) is 2-100,000 fold lower than the parental (e.g., wild-type) mucin-active protease. In some embodiments, the effective substrate Kd of the mucin-active protease is in the micromolar range. In certain embodiments, the targeting moiety exhibits target affinity (measured, e.g., by effective Kd) 2-100,000 fold higher than the mucin-active protease's substrate affinity. According to some embodiments, the targeting moiety exhibits an effective target Kd in the nanomolar range.

As summarized above, the mucin-active protease is stably associated with the targeting moiety. By “stably associated” is meant a physical association between two entities in which the mean half-life of association is one day or more in phosphate buffered saline (PBS) at 4° C. In some embodiments, the physical association between the two entities has a mean half-life of one day or more, one week or more, one month or more, including six months or more, e.g., 1 year or more, in PBS at 4° C. According to some embodiments, the stable association arises from a covalent bond between the two entities, a non-covalent bond between the two entities (e.g., an ionic or metallic bond), or other forms of chemical attraction, such as hydrogen bonding, Van der Waals forces, and the like.

In certain embodiments, the mucin-active protease is stably associated with the targeting moiety via fusion of a protein domain comprising the mucin-active protease and a protein domain comprising the targeting moiety. In other words, the mucin-active protease may be part of a fusion protein comprising the mucin-active protease fused directly or indirectly to the targeting moiety. According to some embodiments, the protein domain comprising the mucin-active protease is fused indirectly via a linker to the protein domain comprising the targeting moiety. Non-limiting examples of a linker that may be employed include a glycine-serine linker. A non-limiting example of a fusion protein comprising a mucin-active protease fused to a targeting moiety is provided in the Experimental section below.

According to some embodiments, the mucin-active protease is stably associated with the targeting moiety via conjugation. The term “conjugation” or “conjugated” generally refers to a chemical linkage, either covalent or non-covalent, usually covalent, that proximally associates one molecule of interest with a second molecule of interest. In certain embodiments, the mucin-active protease is conjugated to the targeting moiety via a linker. If present, the linker molecule(s) may be of sufficient length to permit the mucin-active protease and targeting moiety to allow some flexible movement between the mucin-active protease and targeting moiety. Linker molecules may be, e.g., about 6-50 atoms long. Linker molecules may also be, e.g., aryl acetylene, ethylene glycol oligomers containing 2-10 monomer units, diamines, diacids, amino acids, or combinations thereof.

Where the linkers are peptides, the linkers can be of any suitable length, such as from 1 amino acid (e.g., Gly) to 20 or more amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids, and may be 1, 2, 3, 4, 5, 6, or 7 amino acids in length.

Flexible linkers include glycine polymers (G) n, glycine-serine polymers, glycine-alanine polymers, alanine-serine polymers, and other flexible linkers known in the art. Glycine and glycine-serine polymers may be used where relatively unstructured amino acids are of interest, and may serve as a neutral tether between components. The ordinarily skilled artisan will recognize that design of conjugates can include linkers that are all or partially flexible, such that the linker can include a flexible linker as well as one or more portions that confer a less flexible structure.

According to some embodiments, the mucin-active protease is conjugated to the targeting moiety via a non-cleavable linker. Non-cleavable linkers of interest include, but are not limited to, thioether linkers. An example of a thioether linker that may be employed includes a succinimidyl 4-(N-maleimidomethyl)cyclohexane-1-carboxylate (SMCC) linker.

In certain embodiments, the mucin-active protease is conjugated to the targeting moiety via a cleavable linker. According to some embodiments, the linker is a chemically-labile linker, such as an acid-cleavable linker that is stable at neutral pH (bloodstream pH 7.3-7.5) but undergoes hydrolysis upon internalization into the mildly acidic endosomes (pH 5.0-6.5) and lysosomes (pH 4.5-5.0) of a target cell (e.g., a cancer cell). Chemically-labile linkers include, but are not limited to, hydrazone-based linkers, oxime-based linkers, carbonate-based linkers, ester-based linkers, etc. In certain embodiments, the linker is an enzyme-labile linker, such as an enzyme-labile linker that is stable in the bloodstream but undergoes enzymatic cleavage upon internalization into a target cell, e.g., by a lysosomal protease (such as cathepsin or plasmin) in a lysosome of the target cell (e.g., a cancer cell). Enzyme-labile linkers include, but are not limited to, linkers that include peptidic bonds, e.g., dipeptide-based linkers such as valine-citrulline (VC) linkers, such as a maleimidocaproyl-valine-citruline-p-aminobenzyl (MC-vc-PAB) linker, a valyl-alanyl-para-aminobenzyloxy (Val-Ala-PAB) linker, and the like. Chemically-labile linkers, enzyme-labile, and non-cleavable linkers are known and described in detail, e.g., in Ducry & Stump (2010) Bioconjugate Chem. 21:5-13; Nolting, B. (2013) Methods Mol Biol. 1045:71-100; Tsuchikama and An (2018) Protein & Cell 9 (1): 33-46; and elsewhere.

Numerous strategies are available for linking the mucin-active protease and targeting moiety directly, or indirectly via a linker. For example, the mucin-active protease may be derivatized by covalently attaching a linker to the mucin-active protease, where the linker has a functional group capable of reacting with a “chemical handle” on the targeting moiety. Also by way of example, the targeting moiety may be derivatized by covalently attaching a linker to the targeting moiety, where the linker has a functional group capable of reacting with a “chemical handle” on the mucin-active protease. The functional group on the linker may vary and may be selected based on compatibility with the chemical handle on the mucin-active protease or targeting moiety. According to one embodiment, the chemical handle is provided by incorporation of an unnatural amino acid having the chemical handle into the mucin-active protease or targeting moiety. Unnatural amino acids which find use for preparing the conjugates of the present disclosure include those having a functional group selected from an azide, alkyne, alkene, amino-oxy, hydrazine, aldehyde (e.g., formylglycine, e.g., SMARTag™ technology from Catalent Pharma Solutions), nitrone, nitrile oxide, cyclopropene, norbornene, iso-cyanide, aryl halide, and boronic acid functional group. Unnatural amino acids which may be incorporated into a mucin-active protease or targeting moiety of a conjugate of the present disclosure, which unnatural amino acid may be selected to provide a functional group of interest, are known and described in, e.g., Maza et al. (2015) Bioconjug. Chem. 26 (9): 1884-9; Patterson et al. (2014) ACS Chem. Biol. 9:592-605; Adumeau et al. (2016) Mol. Imaging Biol. (2): 153-65; and elsewhere. An unnatural amino acid may be incorporated into a mucin-active protease or targeting moiety via chemical synthesis or recombinant approaches, e.g., using a suitable orthogonal amino acyl tRNA synthetase-tRNA pair for incorporation of the unnatural amino acid during translation of the a mucin-active protease or targeting moiety in a host cell.

The functional group of an unnatural amino acid present in the mucin-active protease or targeting moiety may be an azide, alkyne, alkene, amino-oxy, hydrazine, aldehyde, asaldehyde, nitrone, nitrile oxide, cyclopropene, norbornene, iso-cyanide, aryl halide, boronic acid, diazo, tetrazine, tetrazole, quadrocyclane, iodobenzene, or other suitable functional group, and the functional group on the linker is selected to react with the functional group of the unnatural amino acid (or vice versa). As just one example, an azide-bearing unnatural amino acid (e.g., 5-azido-L-norvaline, or the like) may be incorporated into the mucin-active protease or targeting moiety and the linker portion of a linker-agent moiety may include an alkyne functional group, such that the mucin-active protease or targeting moiety and linker-agent moiety are covalently conjugated via azide-alkyne cycloaddition. Conjugation may be carried out using, e.g., a copper-catalyzed azide-alkyne cycloaddition reaction.

In certain embodiments, the chemical handle on the mucin-active protease or targeting moiety does not involve an unnatural amino acid. A mucin-active protease or targeting moiety containing no unnatural amino acids may be conjugated by utilizing, e.g., nucleophilic functional groups of the mucin-active protease or targeting moiety (such as the N-terminal amine or the primary amine of lysine, or any other nucleophilic amino acid residue) as a nucleophile in a substitution reaction with a moiety bearing a reactive leaving group or other electrophilic group. An example would be to prepare a mucin-active protease-linker moiety bearing an N-hydroxysuccinimidyl (NHS) ester and allow it to react with the targeting moiety under aqueous conditions at elevated pH (˜10) or in polar organic solvents such as DMSO with an added non-nucleophilic base, such as N,N-diisopropylethylamine.

It will be appreciated that the particular approach for attaching a linker, mucin-active protease and/or targeting moiety to each other may vary depending upon the particular linker, mucin-active protease and/or targeting moiety and functional groups selected and employed for conjugating the various components to each other.

### Methods of Producing Mucin-Active Proteases

Using the information provided herein, the mucin-active proteases and fusion proteins of the present disclosure may be prepared using standard techniques well known to those of skill in the art. For example, a nucleic acid sequence(s) encoding the amino acid sequence of a mucin-active protease of the present disclosure can be used to express the mucin-active proteases or fusion proteins. The polypeptide sequences provided herein (see, e.g., Tables 1 and 2) can be used to determine appropriate nucleic acid sequences encoding the mucin-active proteases or fusion proteins and the nucleic acids sequences then used to express one or more mucin-active proteases or fusion proteins. The nucleic acid sequence(s) can be optimized to reflect particular codon “preferences” for various expression systems according to standard methods well known to those of skill in the art. Using the sequence information provided, the nucleic acids may be synthesized according to a number of standard methods known to those of skill in the art.

Once a nucleic acid(s) encoding a subject mucin-active protease or fusion protein is synthesized, it can be amplified and/or cloned according to standard methods. Molecular cloning techniques to achieve these ends are known in the art. A wide variety of cloning and in vitro amplification methods suitable for the construction of recombinant nucleic acids are known to persons of skill in the art and are the subjects of numerous textbooks and laboratory manuals.

Expression of natural or synthetic nucleic acids encoding the mucin-active proteases or fusion proteins of the present disclosure can be achieved by operably linking a nucleic acid encoding the mucin-active protease or fusion protein to a promoter (which is either constitutive or inducible), and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the mucin-active protease or fusion protein. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression plasmids which typically contain a strong promoter to direct transcription, a ribosome binding site for translational initiation, and a transcription/translation terminator, each in functional orientation to each other and to the protein-encoding sequence. Examples of regulatory regions suitable for this purpose in E. coli are the promoter and operator region of the E. coli tryptophan biosynthetic pathway, the leftward promoter of phage lambda (PL), and the L-arabinose (araBAD) operon. The inclusion of selection markers in DNA vectors transformed in E. coli is also useful. Examples of such markers include genes specifying resistance to ampicillin, tetracycline, or chloramphenicol. Expression systems for expressing mucin-active proteases or fusion proteins are available using, for example, E. coli, Bacillus sp. and Salmonella. E. coli systems may also be used.

The mucin-active protease or fusion protein gene(s) may also be subcloned into an expression vector that allows for the addition of a tag (e.g., FLAG, his (e.g., hexahistidine), and the like) at the C-terminal end or the N-terminal end of the mucin-active protease or fusion protein to facilitate purification. Methods of transfecting and expressing genes in mammalian cells are known in the art. Transducing cells with nucleic acids can involve, for example, incubating lipidic microparticles containing nucleic acids with cells or incubating viral vectors containing nucleic acids with cells within the host range of the vector. The culture of cells used in the present disclosure, including cell lines and cultured cells from tissue (e.g., tumor) or blood samples is well known in the art.

Once the nucleic acid encoding a subject mucin-active protease or fusion protein is isolated and cloned, one can express the nucleic acid in a variety of recombinantly engineered cells known to those of skill in the art. Examples of such cells include bacteria, yeast, filamentous fungi, insect (e.g., those employing baculoviral vectors), and mammalian cells.

Isolation and purification of a subject mucin-active protease or fusion protein can be accomplished according to methods known in the art. For example, a protein can be isolated from a lysate of cells genetically modified to express the protein constitutively and/or upon induction, or from a synthetic reaction mixture, by immunoaffinity purification (or precipitation using Protein L or A), washing to remove non-specifically bound material, and eluting the specifically bound mucin-active protease or fusion protein. The isolated mucin-active protease or fusion protein can be further purified by dialysis and other methods normally employed in protein purification methods. In one embodiment, the mucin-active protease or fusion protein may be isolated using metal chelate chromatography methods. Mucin-active proteases and fusion proteins of the present disclosure may contain modifications to facilitate isolation, as discussed elsewhere herein.

The mucin-active proteases or fusion proteins may be prepared in substantially pure or isolated form (e.g., free from other polypeptides). The protein can be present in a composition that is enriched for the polypeptide relative to other components that may be present (e.g., other polypeptides or other host cell components). Purified mucin-active proteases or fusion proteins may be provided such that the mucin-active protease or fusion protein is present in a composition that is substantially free of other expressed proteins, e.g., less than 90%, usually less than 60% and more usually less than 50% of the composition is made up of other expressed proteins.

The mucin-active proteases or fusion proteins produced by prokaryotic cells may require exposure to chaotropic agents for proper folding. During purification from E. coli, for example, the expressed protein can be optionally denatured and then renatured. This can be accomplished, e.g., by solubilizing the bacterially produced mucin-active proteases or fusion proteins in a chaotropic agent such as guanidine HCl. The mucin-active protease or fusion protein is then renatured, either by slow dialysis or by gel filtration. Alternatively, nucleic acid encoding the mucin-active protease or fusion protein may be operably linked to a secretion signal sequence such as pelB so that the mucin-active proteases or fusion proteins are secreted into the periplasm in correctly-folded form.

The present disclosure also provides cells that produce the mucin-active proteases or fusion proteins of the present disclosure, where suitable cells include eukaryotic cells (e.g., mammalian cells) and prokaryotic cells, e.g., bacterial cells. When bacterial cells are employed, in some embodiments, endotoxin is removed from the mucin-active protease or fusion protein subsequent to expression, and/or the bacterial cells are genetically modified such that they do not produce endotoxin. The present disclosure provides a recombinant host cell (also referred to herein as a “genetically modified host cell”) that is genetically modified with one or more nucleic acids comprising a nucleotide sequence encoding a mucin-active protease or fusion protein of the present disclosure.

### Nucleic Acids, Expression Vectors and Cells

In view of the section above regarding methods of producing the mucin-active proteases and fusion proteins of the present disclosure, it will be appreciated that the present disclosure also provides nucleic acids, expression vectors and cells.

According to some embodiments, provided is a nucleic acid encoding a mucin-active protease or fusion protein of the present disclosure. In certain embodiments, the mucin-active protease is stably associated with the targeting moiety via fusion of a protein domain comprising the mucin-active protease and a protein domain comprising the targeting moiety, and where the nucleic acid encodes the protein domain comprising the mucin-active protease fused to the protein domain comprising the targeting moiety.

Also provided are expression vectors comprising any of the nucleic acids of the present disclosure. Expression of natural or synthetic nucleic acids encoding the mucin-active proteases and fusion proteins of the present disclosure can be achieved by operably linking a nucleic acid encoding the mucin-active protease or fusion protein to a promoter (which is either constitutive or inducible) and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the mucin-active protease or fusion protein. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

Cells that comprise any of the nucleic acids and/or expression vectors of the present disclosure are also provided. Also provided are methods of making a mucin-active protease or fusion protein of the present disclosure, the methods including culturing a cell of the present disclosure under conditions suitable for the cell to express the mucin-active protease or fusion protein, where the mucin-active protease or fusion protein is produced. The conditions for culturing the cell such that the mucin-active protease or fusion protein is expressed may vary. Such conditions may include culturing the cell in a suitable container (e.g., a cell culture plate or well thereof), in suitable medium (e.g., cell culture medium, such as DMEM, RPMI, MEM, IMDM, DMEM/F-12, or the like) at a suitable temperature (e.g., 32° C.-42° C., such as 37° C.) and pH (e.g., pH 7.0-7.7, such as pH 7.4) in an environment having a suitable percentage of CO₂, e.g., 3% to 10%, such as 5%).

### Compositions

As summarized above, the present disclosure also provides compositions. According to some embodiments, a composition of the present disclosure includes a mucin-active protease of the present disclosure, e.g., a mucin-active protease fused or conjugated to a targeting moiety. For example, the mucin-active protease may be any of the mucin-active proteases described in the Mucin-Active Proteases section hereinabove or in the Experimental section below, which descriptions are incorporated but not reiterated herein for purposes of brevity.

In certain aspects, a composition of the present disclosure includes the mucin-active protease present in a liquid medium. The liquid medium may be an aqueous liquid medium, such as water, a buffered solution, or the like. One or more additives such as a salt (e.g., NaCl, MgCl₂, KCl, MgSO₄), a buffering agent (a Tris buffer, N-(2-Hydroxyethyl) piperazine-N′-(2-ethanesulfonic acid) (HEPES), 2-(N-Morpholino) ethanesulfonic acid (MES), 2-(N-Morpholino) ethanesulfonic acid (MES), sodium salt 3-(N-Morpholino) propanesulfonic acid (MOPS), N-tris[Hydroxymethyl]methyl-3-aminopropanesulfonic acid (TAPS), etc.), a solubilizing agent, a detergent (e.g., a non-ionic detergent such as Tween-20, etc.), a nuclease inhibitor, a protease inhibitor, glycerol, a chelating agent, and the like may be present in such compositions.

Aspects of the present disclosure further include pharmaceutical compositions. In some embodiments, a pharmaceutical composition of the present disclosure includes a mucin-active protease of the present disclosure, and a pharmaceutically acceptable carrier.

The mucin-active protease can be incorporated into a variety of formulations for therapeutic administration. More particularly, the mucin-active proteases can be formulated into pharmaceutical compositions by combination with appropriate, pharmaceutically acceptable excipients or diluents, and may be formulated into preparations in solid, semi-solid, liquid or gaseous forms, such as tablets, capsules, powders, granules, ointments, solutions, injections, inhalants and aerosols.

Formulations of the mucin-active proteases for administration to an individual (e.g., suitable for human administration) are generally sterile and may further be free of detectable pyrogens or other contaminants contraindicated for administration to a patient according to a selected route of administration.

In pharmaceutical dosage forms, the mucin-active proteases can be administered in the form of their pharmaceutically acceptable salts, or they may also be used alone or in appropriate association, as well as in combination, with other pharmaceutically active compounds. The following methods and carriers/excipients are merely examples and are in no way limiting.

For oral preparations, the mucin-active proteases can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The mucin-active proteases can be formulated for parenteral (e.g., intravenous, intra-arterial, intraosseous, intramuscular, intracerebral, intracerebroventricular, intrathecal, subcutaneous, etc.) administration. In certain aspects, the mucin-active proteases are formulated for injection by dissolving, suspending or emulsifying the mucin-active proteases in an aqueous or non-aqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Pharmaceutical compositions that include the mucin-active proteases may be prepared by mixing the mucin-active proteases having the desired degree of purity with optional physiologically acceptable carriers, excipients, stabilizers, surfactants, buffers and/or tonicity agents. Acceptable carriers, excipients and/or stabilizers are nontoxic to recipients at the dosages and concentrations employed, and include buffers such as phosphate, citrate, and other organic acids; antioxidants including ascorbic acid, glutathione, cysteine, methionine and citric acid; preservatives (such as ethanol, benzyl alcohol, phenol, m-cresol, p-chlor-m-cresol, methyl or propyl parabens, benzalkonium chloride, or combinations thereof); amino acids such as arginine, glycine, ornithine, lysine, histidine, glutamic acid, aspartic acid, isoleucine, leucine, alanine, phenylalanine, tyrosine, tryptophan, methionine, serine, proline and combinations thereof; monosaccharides, disaccharides and other carbohydrates; low molecular weight (less than about 10 residues) polypeptides; proteins, such as gelatin or serum albumin; chelating agents such as EDTA; sugars such as trehalose, sucrose, lactose, glucose, mannose, maltose, galactose, fructose, sorbose, raffinose, glucosamine, N-methylglucosamine, galactosamine, and neuraminic acid; and/or non-ionic surfactants such as Tween, Brij Pluronics, Triton-X, or polyethylene glycol (PEG).

The pharmaceutical composition may be in a liquid form, a lyophilized form or a liquid form reconstituted from a lyophilized form, wherein the lyophilized preparation is to be reconstituted with a sterile solution prior to administration. The standard procedure for reconstituting a lyophilized composition is to add back a volume of pure water (typically equivalent to the volume removed during lyophilization); however solutions comprising antibacterial agents may be used for the production of pharmaceutical compositions for parenteral administration.

An aqueous formulation of the mucin-active proteases may be prepared in a pH-buffered solution, e.g., at pH ranging from about 4.0 to about 7.0, or from about 5.0 to about 6.0, or alternatively about 5.5. Examples of buffers that are suitable for a pH within this range include phosphate-, histidine-, citrate-, succinate-, acetate-buffers and other organic acid buffers. The buffer concentration can be from about 1 mM to about 100 mM, or from about 5 mM to about 50 mM, depending, e.g., on the buffer and the desired tonicity of the formulation.

A tonicity agent may be included to modulate the tonicity of the formulation. Example tonicity agents include sodium chloride, potassium chloride, glycerin and any component from the group of amino acids, sugars as well as combinations thereof. In some embodiments, the aqueous formulation is isotonic, although hypertonic or hypotonic solutions may be suitable. The term “isotonic” denotes a solution having the same tonicity as some other solution with which it is compared, such as physiological salt solution or serum. Tonicity agents may be used in an amount of about 5 mM to about 350 mM, e.g., in an amount of 100 mM to 350 mM.

A surfactant may also be added to the formulation to reduce aggregation and/or minimize the formation of particulates in the formulation and/or reduce adsorption. Example surfactants include polyoxyethylensorbitan fatty acid esters (Tween), polyoxyethylene alkyl ethers (Brij), alkylphenylpolyoxyethylene ethers (Triton-X), polyoxyethylene-polyoxypropylene copolymer (Poloxamer, Pluronic), and sodium dodecyl sulfate (SDS). Examples of suitable polyoxyethylenesorbitan-fatty acid esters are polysorbate 20, (sold under the trademark Tween 20™) and polysorbate 80 (sold under the trademark Tween 80™). Examples of suitable polyethylene-polypropylene copolymers are those sold under the names Pluronic® F68 or Poloxamer 188™. Examples of suitable Polyoxyethylene alkyl ethers are those sold under the trademark Brij™. Example concentrations of surfactant may range from about 0.001% to about 1% w/V.

A lyoprotectant may also be added in order to protect the mucin-active proteases against destabilizing conditions during a lyophilization process. For example, known lyoprotectants include sugars (including glucose and sucrose); polyols (including mannitol, sorbitol and glycerol); and amino acids (including alanine, glycine and glutamic acid). Lyoprotectants can be included, e.g., in an amount of about 10 mM to 500 nM.

In some embodiments, the pharmaceutical composition includes the mucin-active protease, and one or more of the above-identified components (e.g., a surfactant, a buffer, a stabilizer, a tonicity agent) and is essentially free of one or more preservatives, such as ethanol, benzyl alcohol, phenol, m-cresol, p-chlor-m-cresol, methyl or propyl parabens, benzalkonium chloride, and combinations thereof. In other embodiments, a preservative is included in the formulation, e.g., at concentrations ranging from about 0.001 to about 2% weight/volume (w/v).

### Methods of Use

The present disclosure provides methods of using the mucin-active proteases of the present disclosure. For example, in certain embodiments, provided are methods of treating a mucin-associated condition in a subject in need thereof. Such methods comprise administering to the subject an effective amount of a mucin-active protease of the present disclosure, wherein upon administration of the mucin-active protease to the subject, the targeting moiety targets the mucin-active protease to cell surface, extracellular and/or secreted mucins. In certain embodiments, the targeting moiety targets the mucin-active protease to target cells comprising cell surface mucins, and the mucin-active protease degrades the cells surface mucins.

In certain embodiments, the targeting moiety binds to a cell surface molecule of a target cell, where the target cell comprises cell surface mucin domain-containing glycoproteins, and where it is desirable to cleave the cell surface mucin domain-containing glycoproteins using the mucin-active protease, e.g., for therapeutic purposes. In other words, the targeting moiety provides for degradation of cell surface, extracellular and/or secreted mucin domain-containing glycoproteins by the mucin-active protease in a targeted manner in vivo to treat a mucin-associated condition. According to some embodiments, the targeting moiety binds to an extracellular and/or secreted molecule (e.g., an extracellular and/or secreted mucin domain-containing glycoprotein, or an extracellular and/or secreted molecule which colocalizes with extracellular and/or secreted mucin domain-containing glycoproteins), and where it is desirable to cleave the extracellular and/or secreted mucin domain-containing glycoproteins using the mucin-active protease, e.g., for therapeutic purposes. That is, the targeting moiety provides for degradation of extracellular and/or secreted mucin domain-containing glycoproteins by the mucin-active protease in a targeted manner in vivo to treat a mucin-associated condition.

In certain embodiments, the mucin-associated condition is a cell proliferative disorder. By “cell proliferative disorder” is meant a disorder wherein unwanted cell proliferation of one or more subset(s) of cells in a multicellular organism occurs, resulting in harm, for example, pain or decreased life expectancy to the organism. Cell proliferative disorders include, but are not limited to, cancer, pre-cancer, benign tumors, blood vessel proliferative disorders (e.g., arthritis, restenosis, and the like), fibrotic disorders (e.g., hepatic cirrhosis, atherosclerosis, and the like), psoriasis, epidermic and dermoid cysts, lipomas, adenomas, capillary and cutaneous hemangiomas, lymphangiomas, nevi lesions, teratomas, nephromas, myofibromatosis, osteoplastic tumors, dysplastic masses, mesangial cell proliferative disorders, and the like.

In some embodiments, the mucin-associated condition is cancer. The subject methods may be employed for the treatment of a large variety of cancers. “Tumor”, as used herein, refers to all neoplastic cell growth and proliferation, whether malignant or benign, and all pre-cancerous and cancerous cells and tissues. The terms “cancer” and “cancerous” refer to or describe the physiological condition in mammals that is typically characterized by unregulated cell growth/proliferation. Examples of cancers that may be treated using the subject methods include, but are not limited to, carcinoma, lymphoma, blastoma, and sarcoma. More particular examples of such cancers include squamous cell cancer, small-cell lung cancer, non-small cell lung cancer, adenocarcinoma of the lung, squamous carcinoma of the lung, cancer of the peritoneum, hepatocellular cancer, gastrointestinal cancer, pancreatic cancer, glioblastoma, cervical cancer, ovarian cancer, liver cancer, bile duct cancer, bladder cancer, hepatoma, breast cancer, colon cancer, colorectal cancer, endometrial or uterine carcinoma, salivary gland carcinoma, kidney cancer, prostate cancer, vulval cancer, thyroid cancer, hepatic carcinoma, various types of head and neck cancer, and the like. In certain embodiments, the individual has a cancer selected from a solid tumor, recurrent glioblastoma multiforme (GBM), non-small cell lung cancer, metastatic melanoma, melanoma, peritoneal cancer, epithelial ovarian cancer, glioblastoma multiforme (GBM), metastatic colorectal cancer, colorectal cancer, pancreatic ductal adenocarcinoma, squamous cell carcinoma, esophageal cancer, gastric cancer, neuroblastoma, fallopian tube cancer, bladder cancer, metastatic breast cancer, pancreatic cancer, soft tissue sarcoma, recurrent head and neck cancer squamous cell carcinoma, head and neck cancer, anaplastic astrocytoma, malignant pleural mesothelioma, squamous non-small cell lung cancer, rhabdomyosarcoma, metastatic renal cell carcinoma, basal cell carcinoma (basal cell epithelioma), and gliosarcoma. In certain aspects, the individual has a cancer selected from melanoma, Hodgkin lymphoma, renal cell carcinoma (RCC), bladder cancer, non-small cell lung cancer (NSCLC), and head and neck squamous cell carcinoma (HNSCC).

In certain embodiments, the mucin-associated condition is cancer, and the cancer comprises a solid tumor. According to some embodiments, the solid tumor is a carcinoma or a sarcoma. When the solid tumor is a carcinoma, in certain embodiments, the carcinoma is a basal cell carcinoma, squamous cell carcinoma, renal cell carcinoma, ductal carcinoma in situ (DCIS), invasive ductal carcinoma, or adenocarcinoma. According to some embodiments, when the cancer comprises a solid tumor, the solid tumor is immune-infiltrated. In certain embodiments, the mucin-associated condition is cancer, and the cancer is a myeloma, a leukemia, a lymphoma, or mixed type. In certain embodiments, when the mucin-associated condition is cancer, the cancer is susceptible to mechanical stress. According to some embodiments, when the mucin-associated condition is cancer, the cancer is sensitive to ferroptosis. In certain embodiments, when the mucin-associated condition is cancer, the cancer is of a mucinous subtype. By “mucinous subtype” is meant individual cancer cells are suspended in a secreted matrix of polysaccharides and glycoproteins.

The methods of the present disclosure may be used to treat a variety of other mucin-associated conditions, non-limiting examples of which include viral infection (e.g., a respiratory virus infection), cystic fibrosis, bacterial endocarditis and/or gut dysbiosis.

The mucin-active proteases of the present disclosure may be administered via a route of administration selected from oral (e.g., in tablet form, capsule form, liquid form, or the like), parenteral (e.g., by intravenous, intra-arterial, subcutaneous, intramuscular, or epidural injection), topical, intra-nasal, intra-tumoral administration, or intraperitoneal (IP) administration.

The mucin-active proteases of the present disclosure may be administered (e.g., in a pharmaceutical composition) in a therapeutically effective amount. By “therapeutically effective amount” is meant a dosage sufficient to produce a desired result, e.g., an amount sufficient to effect beneficial or desired therapeutic (including preventative) results, such as a reduction in a symptom of a cancer (e.g., a carcinoma), viral infection, cystic fibrosis, bacterial endocarditis and/or gut dysbiosis as compared to a control. With respect to cancer, in some embodiments, the therapeutically effective amount is sufficient to slow the growth of a tumor, reduce the size of a tumor, and/or the like. An effective amount can be administered in one or more administrations.

Provided are methods of treating a mucin-associated condition in a subject in need thereof. By treatment is meant at least an amelioration of one or more symptoms associated with the mucin-associated condition of the individual, where amelioration is used in a broad sense to refer to at least a reduction in the magnitude of a parameter, e.g., symptom, associated with the mucin-associated condition being treated. As such, treatment also includes situations where the mucin-associated condition, or at least one or more symptoms associated therewith, are completely inhibited, e.g., prevented from happening, or stopped, e.g., terminated, such that the individual no longer suffers from the mucin-associated condition, or at least the symptoms that characterize the mucin-associated condition.

A mucin-active protease of the present disclosure may be administered to the individual alone or in combination with a second agent. Second agents of interest include, but are not limited to, agents approved by the United States Food and Drug Administration and/or the European Medicines Agency (EMA) for use in treating cancer. In some embodiments, the second agent is an immune checkpoint inhibitor. Immune checkpoint inhibitors of interest include, but are not limited to, a cytotoxic T-lymphocyte-associated antigen 4 (CTLA-4) inhibitor, a programmed cell death-1 (PD-1) inhibitor, a programmed cell death ligand-1 (PD-L1) inhibitor, a lymphocyte activation gene-3 (LAG-3) inhibitor, a T-cell immunoglobulin domain and mucin domain 3 (TIM-3) inhibitor, an indoleamine (2,3)-dioxygenase (IDO) inhibitor, a T cell immunoreceptor with Ig and ITIM domains (TIGIT) inhibitor, a V-domain Ig suppressor of T cell activation (VISTA) inhibitor, a B7-H3 inhibitor, and any combination thereof.

When a mucin-active protease of the present disclosure is administered with a second agent, the mucin-active protease and the second agent may be administered to the individual according to any suitable administration regimen. According to certain embodiments, the mucin-active protease and the second agent are administered according to a dosing regimen approved for individual use. In some embodiments, the administration of the mucin-active protease permits the second agent to be administered according to a dosing regimen that involves one or more lower and/or less frequent doses, and/or a reduced number of cycles as compared with that utilized when the second agent is administered without administration of the mucin-active protease. In certain aspects, the administration of the second agent permits the mucin-active protease to be administered according to a dosing regimen that involves one or more lower and/or less frequent doses, and/or a reduced number of cycles as compared with that utilized when the mucin-active protease is administered without administration of the second agent.

In some embodiments, one or more doses of the mucin-active protease and the second agent are administered concurrently to the individual. By “concurrently” is meant the mucin-active protease and the second agent are either present in the same pharmaceutical composition, or the mucin-active protease and the second agent are administered as separate pharmaceutical compositions within 1 hour or less, 30 minutes or less, or 15 minutes or less.

In some embodiments, one or more doses of the mucin-active protease and the second agent are administered sequentially to the individual.

In some embodiments, the mucin-active protease and the second agent are administered to the individual in different compositions and/or at different times. For example, the mucin-active protease may be administered prior to administration of the second agent, e.g., in a particular cycle. Alternatively, the second agent may be administered prior to administration of the mucin-active protease, e.g., in a particular cycle. The second agent to be administered may be administered a period of time that starts at least 1 hour, 3 hours, 6 hours, 12 hours, 24 hours, 48 hours, 72 hours, or up to 5 days or more after the administration of the first agent to be administered.

In one example, the second agent is administered to the individual for a desirable period of time prior to administration of the mucin-active protease. In certain aspects, when the individual has cancer, such a regimen “primes” the cancer cells to potentiate the anti-cancer effect of the mucin-active protease. Such a period of time separating a step of administering the second agent from a step of administering the mucin-active protease is of sufficient length to permit priming of the cancer cells, desirably so that the anti-cancer effect of the mucin-active protease is increased.

In some embodiments, administration of one agent is specifically timed relative to administration of the other agent. For example, in some embodiments, the mucin-active protease is administered so that a particular effect is observed (or expected to be observed, for example based on population studies showing a correlation between a given dosing regimen and the particular effect of interest).

In certain aspects, desired relative dosing regimens for agents administered in combination may be assessed or determined empirically, for example using ex vivo, in vivo and/or in vitro models; in some embodiments, such assessment or empirical determination is made in vivo, in a patient population (e.g., so that a correlation is established), or alternatively in a particular individual of interest.

In some embodiments, the mucin-active protease and the second agent are administered according to an intermittent dosing regimen including at least two cycles. Where two or more agents are administered in combination, and each by such an intermittent, cycling, regimen, individual doses of different agents may be interdigitated with one another. In certain aspects, one or more doses of a second agent is administered a period of time after a dose of the first agent. In some embodiments, each dose of the second agent is administered a period of time after a dose of the first agent. In certain aspects, each dose of the first agent is followed after a period of time by a dose of the second agent. In some embodiments, two or more doses of the first agent are administered between at least one pair of doses of the second agent; in certain aspects, two or more doses of the second agent are administered between at least one pair of doses of the first agent. In some embodiments, different doses of the same agent are separated by a common interval of time; in some embodiments, the interval of time between different doses of the same agent varies. In certain aspects, different doses of the mucin-active protease and the second agent are separated from one another by a common interval of time; in some embodiments, different doses of the different agents are separated from one another by different intervals of time.

One exemplary protocol for interdigitating two intermittent, cycled dosing regimens may include: (a) a first dosing period during which a therapeutically effective amount the mucin-active protease is administered to the individual; (b) a first resting period; (c) a second dosing period during which a therapeutically effective amount of the second agent is administered to the individual; and (d) a second resting period. A second exemplary protocol for interdigitating two intermittent, cycled dosing regimens may include: (a) a first dosing period during which a therapeutically effective amount the second agent is administered to the individual; (b) a first resting period; (c) a second dosing period during which a therapeutically effective amount of the mucin-active protease is administered to the individual; and (d) a second resting period.

In some embodiments, the first resting period and second resting period may correspond to an identical number of hours or days. Alternatively, in some embodiments, the first resting period and second resting period are different, with either the first resting period being longer than the second one or, vice versa. In some embodiments, each of the resting periods corresponds to 120 hours, 96 hours, 72 hours, 48 hours, 24 hours, 12 hours, 6 hours, 30 hours, 1 hour, or less. In some embodiments, if the second resting period is longer than the first resting period, it can be defined as a number of days or weeks rather than hours (for instance 1 day, 3 days, 5 days, 1 week, 2, weeks, 4 weeks or more).

If the first resting period's length is determined by existence or development of a particular biological or therapeutic event, then the second resting period's length may be determined on the basis of different factors, separately or in combination. Exemplary such factors may include type and/or stage of a cancer against which the therapy is administered; properties (e.g., pharmacokinetic properties) of the mucin-active protease, and/or one or more features of the patient's response to therapy with the mucin-active protease. In some embodiments, length of one or both resting periods may be adjusted in light of pharmacokinetic properties (e.g., as assessed via plasma concentration levels) of one or the other of the administered agents. For example, a relevant resting period might be deemed to be completed when plasma concentration of the relevant agent is below a pre-determined level, optionally upon evaluation or other consideration of one or more features of the individual's response.

In certain aspects, the number of cycles for which a particular agent is administered may be determined empirically. Also, in some embodiments, the precise regimen followed (e.g., number of doses, spacing of doses (e.g., relative to each other or to another event such as administration of another therapy), amount of doses, etc.) may be different for one or more cycles as compared with one or more other cycles.

The mucin-active protease and the second agent may be administered together or independently via any suitable route of administration. The mucin-active protease and the second agent may be administered via a route of administration independently selected from oral, parenteral (e.g., by intravenous, intra-arterial, subcutaneous, intramuscular, or epidural injection), topical, or intra-nasal administration. According to certain embodiments, the mucin-active protease and the second agent are both administered orally (e.g., in tablet form, capsule form, liquid form, or the like) either concurrently (in the same pharmaceutical composition or separate pharmaceutical compositions) or sequentially.

### Kits

Aspects of the present disclosure further include kits. In certain embodiments, the kits find use in practicing the methods of the present disclosure, e.g., methods of treating a mucin-associated condition in a subject in need thereof.

Accordingly, in certain embodiments, a kit of the present disclosure comprises any of the mucin-active proteases of the present disclosure (e.g., present in a pharmaceutical composition), and instructions for administering the mucin-active protease to an individual in need thereof. As will be appreciated, the kits of the present disclosure may include any of the mucin-active proteases having any of the features (e.g., targeting moieties, etc.) described above in the section relating to the mucin-active proteases of the present disclosure, which are not reiterated herein for purposes of brevity.

The kits of the present disclosure may include a quantity of the mucin-active protease, present in unit dosages, e.g., ampoules, or a multi-dosage format. As such, in certain embodiments, the kits may include one or more (e.g., two or more) unit dosages (e.g., ampoules) of a mucin-active protease of the present disclosure. The term “unit dosage”, as used herein, refers to physically discrete units suitable as unitary dosages for human and animal subjects, each unit containing a predetermined quantity of the mucin-active protease calculated in an amount sufficient to produce the desired effect. The amount of the unit dosage depends on various factors, such as the mucin-active protease employed, the effect to be achieved, and the pharmacodynamics associated with the mucin-active protease, in the individual. In yet other embodiments, the kits may include a single multi dosage amount of the mucin-active protease.

The instructions (e.g., instructions for use (IFU)) included in the kits may be recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g., portable flash drive, etc. In yet other embodiments, the actual instructions are not present in the kit, but means for obtaining the instructions from a remote source (e.g., via the internet) are provided. An example of this embodiment is a kit that includes a web address where the instructions can be viewed and/or from which the instructions can be downloaded. As with the instructions, the means for obtaining the instructions is recorded on a suitable substrate.

Notwithstanding the appended claims, the present disclosure is also defined by the following embodiments:
1. A mucin-active protease stably associated with a targeting moiety.
2. The mucin-active protease of embodiment 1, wherein the mucin-active protease cleaves at a glycan-peptide cleavage motif comprising: S/T *-X-S/T, S/T *-S/T, X-S/T*, S/T *-X, and/or S/T *-X-X-X-X, wherein * denotes glycosylation of the S or T residue and X is any amino acid residue.
3. The mucin-active protease of embodiment 1 or embodiment 2, wherein the mucin-active protease cleaves C1-INH, CADM1, CD43, CD44, CD45, CD68, CXCL1, EMCN, GHA1, GLPA, GLPC, GP1BA, HAVCR1, HEG, MADCAM1, MUC1, MUC2, MUC3A, MUC3B, MUC4, MUC5AC, MUC5B, MUC6, MUC7, MUC8, MUC9, MUC10, MUC11, MUC12, MUC13, MUC14, MUC15, MUC16, MUC17, MUC18, MUC19, MUC20, MUC21, MUC22, MUCL3, PARM1, PILRA, PODXL, PRG4, PSGL1, TIMD4, or a combination thereof.
4. The mucin-active protease of any one of embodiments 1 to 3, wherein the mucin-active protease is a eukaryotic mucin-active protease.
5. The mucin-active protease of any one of embodiments 1 to 3, wherein the mucin-active protease is a prokaryotic mucin-active protease.
6. The mucin-active protease of embodiment 5, wherein the mucin-active protease is a secreted protease of C1 esterase inhibitor (StcE) from Escherichia coli 0157: H7.
7. The mucin-active protease of embodiment 6, wherein the StcE comprises 70% or greater, 75% or greater, 80% or greater, 85% or greater, 90% or greater, 95% or greater, 96% or greater, 97% or greater, 98% or greater, 99% or greater, or 100% amino acid identity with the amino acid sequence set forth in SEQ ID NO:1, or a functional fragment thereof which retains mucin-active protease activity.
8. The mucin-active protease of embodiment 6 or embodiment 7, wherein the StcE comprises one or more deletions relative to the amino acid sequence set forth in SEQ ID NO:1.
9. The mucin-active protease of embodiment 8, wherein the one or more deletions comprises a deletion of all or a portion of the C domain.
10. The mucin-active protease of embodiment 8 or embodiment 9, wherein the one or more deletions comprises a deletion of all or a portion of the INS domain.
11. The mucin-active protease of any one of embodiments 6 to 10, wherein the StcE comprises one or more amino acid substitutions at or near the active site.
12. The mucin-active protease of embodiment 11, wherein the one or more amino acid substitutions comprise a substitution at W366, H367, Y457, or any combination thereof.
13. The mucin-active protease of embodiment 12, wherein the one or more amino acid substitutions comprise W366A, H367A, or both.
14. The mucin-active protease of any one of embodiments 6 to 13, wherein the StcE comprises a deletion of all or a portion of the C domain, a deletion of all or a portion of the INS domain, and a W366A substitution.
15. The mucin-active protease of any one of embodiments 1 to 3, wherein the mucin-active protease is Pic, ZmpB, ZmpC, BT4244, AM0627, AM0908, AM1514, SmEnhancin, VIBHAR2194, CpaA, ImpA, or OgpA.
16. The mucin-active protease of any one of embodiments 1 to 15, wherein the mucin-active protease is a mucin-selective protease.
17. The mucin-active protease of any one of embodiments 1 to 16, wherein the mucin-active protease selectively recognizes a joint glycopeptide epitope.
18. The mucin-active protease of any one of embodiments 1 to 17, wherein the targeting moiety is selected from the group consisting of: a polypeptide, a ligand, an aptamer, a nanoparticle, and a small molecule.
19. The mucin-active protease of embodiment 18, wherein the targeting moiety is a polypeptide.
20. The mucin-active protease of embodiment 19, wherein the targeting moiety comprises an antibody.
21. The mucin-active protease of embodiment 20, wherein the antibody is an IgG, single chain Fv (scFv), Fab, (Fab)₂, (scFv′) 2, or a single variable domain located on a heavy chain (VHH).
22. The mucin-active protease of embodiment 20, wherein the antibody is a VHH.
23. The mucin-active protease of any one of embodiments 1 to 22, wherein the targeting moiety specifically binds a cell surface molecule.
24. The mucin-active protease of any one of embodiments 1 to 23, wherein the targeting moiety specifically binds to a tumor antigen on the surface of the cancer cell.
25. The mucin-active protease of embodiment 24, wherein the tumor antigen is 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvlll, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GD2 ganglioside, glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTCH3, p-cadherin (p-CAD), programmed cell death receptor ligand 1 (PD-L1), programmed cell death receptor ligand 2 (PD-L2), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM⁻¹), Tn antigen, trophoblast cell-surface antigen (TROP-2), Wilms' tumor 1 (WT1), or VEGF-A.
26. The mucin-active protease of any one of embodiments 19 to 25, wherein the mucin-active protease is stably associated with the targeting moiety via fusion of a protein domain comprising the mucin-active protease and a protein domain comprising the targeting moiety.
27. The mucin-active protease of embodiment 26, wherein the protein domain comprising the mucin-active protease is fused via a linker to the protein domain comprising the targeting moiety.
28. The mucin-active protease of embodiment 27, wherein the linker is a glycine-serine linker.
29. The mucin-active protease of any one of embodiments 19 to 25, wherein the mucin-active protease is stably associated with the targeting moiety via conjugation.
30. A nucleic acid encoding the mucin-active protease of any one of embodiments 1 to 29.
31. The nucleic acid of embodiment 30, wherein the mucin-active protease is stably associated with the targeting moiety via fusion of a protein domain comprising the mucin-active protease and a protein domain comprising the targeting moiety, and wherein the nucleic acid encodes the protein domain comprising the mucin-active protease fused to the protein domain comprising the targeting moiety.
32. A cell comprising the nucleic acid of embodiment 30 or embodiment 31.
33. A cell comprising an expression vector comprising the nucleic acid of embodiment 30 or embodiment 31 operably linked to a promoter.
34. A method of producing a mucin-active protease, the method comprising culturing the cell of embodiment 33 under conditions suitable for the cell to express the mucin-active protease, wherein the mucin-active protease is produced.
35. A composition comprising the mucin-active protease of any one of embodiments 1 to 29.
36. A pharmaceutical composition, comprising:
the mucin-active protease of any one of embodiments 1 to 29; and
a pharmaceutically acceptable carrier.
37. A method of treating a mucin-associated condition in a subject in need thereof, the method comprising:
administering to the subject an effective amount of the mucin-active protease of any one of embodiments 1 to 29,
wherein upon administration of the mucin-active protease to the subject, the targeting moiety targets the mucin-active protease to cell surface, extracellular and/or secreted mucins, and the mucin-active protease degrades the mucins.
38. The method according to embodiment 37, wherein the mucin-associated condition is a proliferative disorder.
39. The method according to embodiment 38, wherein the proliferative disorder is cancer.
40. The method according to embodiment 39, wherein the cancer comprises a solid tumor.
41. The method according to embodiment 40, wherein the solid tumor is a carcinoma or a sarcoma.
42. The method according to embodiment 41, wherein the carcinoma is a basal cell carcinoma, squamous cell carcinoma, renal cell carcinoma, ductal carcinoma in situ (DCIS), invasive ductal carcinoma, or adenocarcinoma.
43. The method according to any one of embodiments 40 to 42, wherein the solid tumor is immune-infiltrated.
44. The method according to embodiment 43, wherein the mucin-active protease affects the activation state of immune cells in the tumor microenvironment.
45. The method according to embodiment 44, wherein the mucin-active protease increases activation of tumor infiltrating immune cells.
46. The method according to embodiment 39, wherein the cancer is a myeloma, a leukemia, a lymphoma, or mixed type.
47. The method according to any one of embodiments 39 to 46, wherein the cancer is susceptible to mechanical stress.
48. The method according to any one of embodiments 39 to 47, wherein the cancer is sensitive to ferroptosis.
49. The method according to any one of embodiments 39 to 47, wherein the cancer is of a mucinous subtype.
50. The method according to embodiment 37, wherein the mucin-associated condition is a viral infection.
51. The method according to embodiment 50, wherein the viral infection is a respiratory virus infection.
52. The method according to embodiment 37, wherein the mucin-associated condition is cystic fibrosis.
53. The method according to embodiment 37, wherein the mucin-associated condition is bacterial endocarditis.
54. The method according to embodiment 37, wherein the mucin-associated condition is gut dysbiosis.

The following examples are offered by way of illustration and not by way of limitation.

### Experimental

### Example 1-Mucinase Treatment Undermines Mucin-Driven Survival Pathways in Cancer Cells

Proteases from the bacterial kingdom with selectivity for mucin domains have been characterized¹¹⁻¹⁴. These “mucinases” act through recognition of joint peptide- and glycan-motifs, which have been mapped using mass spectrometry of cleavage products. As an initial candidate for therapeutic repurposing, the zinc metalloprotease StcE from E. coli serotype O157: H7 was chosen. StcE exhibits the motif S/T *-X-S/T, where the first Ser/Thr must bear an O-glycan (asterisk) in order for cleavage to occur¹². StcE is agnostic to the structure of the glycan and the identity of the X amino acid, which can also be absent. StcE is therefore a pan mucinase, able to act upon epitopes present across the natural mucins.

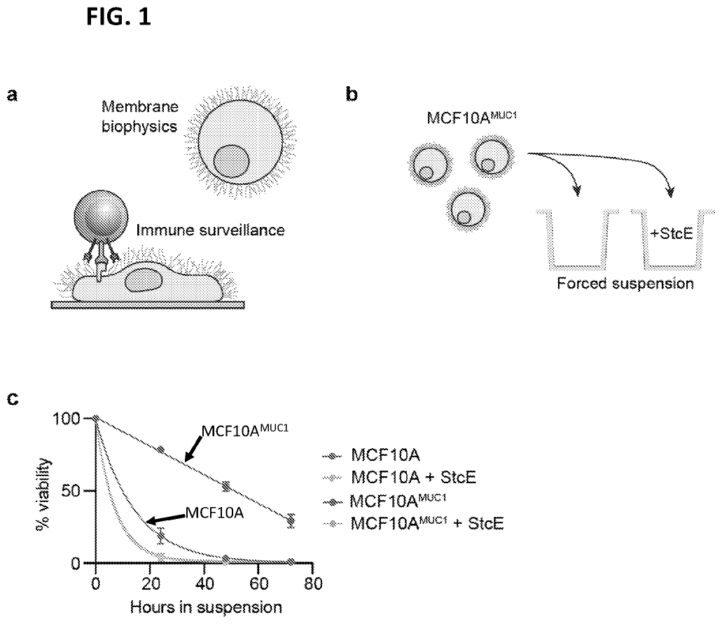

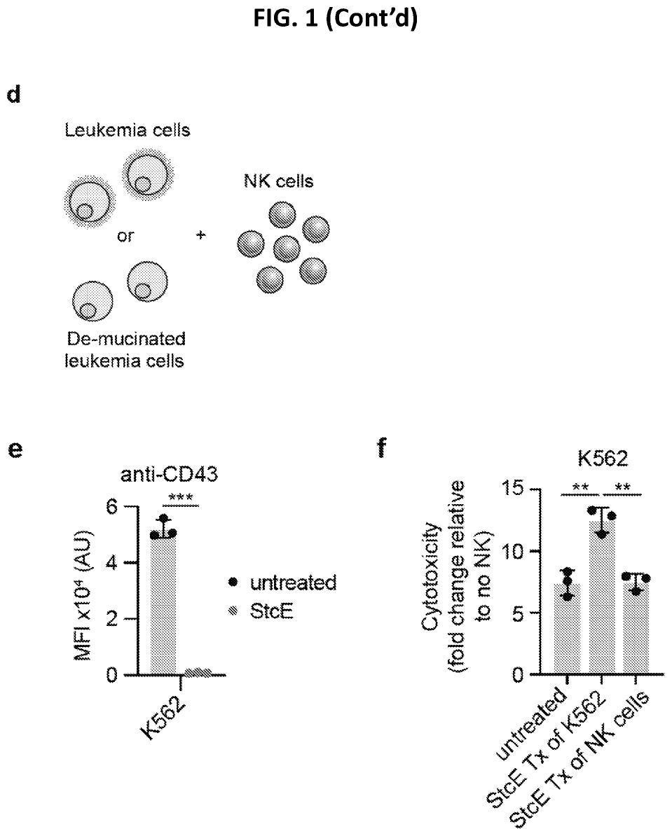

To begin, tested was whether treatment with StcE could reduce cell viability by undermining the biophysical function of mucins. Expression of the MUC1 ectodomain in mammary epithelial cells induces a bulky glycocalyx, which causes the cells to lift from their basement membrane and thrive in suspension in a manner characteristic of circulating metastatic tumor cells¹⁵. To model suspension survival in vitro, wild-type cells and cells overexpressing MUC1 were plated on ultralow attachment plates, treated with or without StcE, and counted by flow cytometry over three days to assess viability (FIG. 1b). Under these anchorage-free conditions, StcE treatment resulted in rapid cell death, consistent with the expected altered membrane biophysics¹⁶ (FIG. 1c). Meanwhile, StcE treatment of MUC1-expressing cells in standard tissue culture plates caused suspended cells to settle, after which they continued to divide, highlighting the low toxicity of mucinase treatment at nanomolar doses.

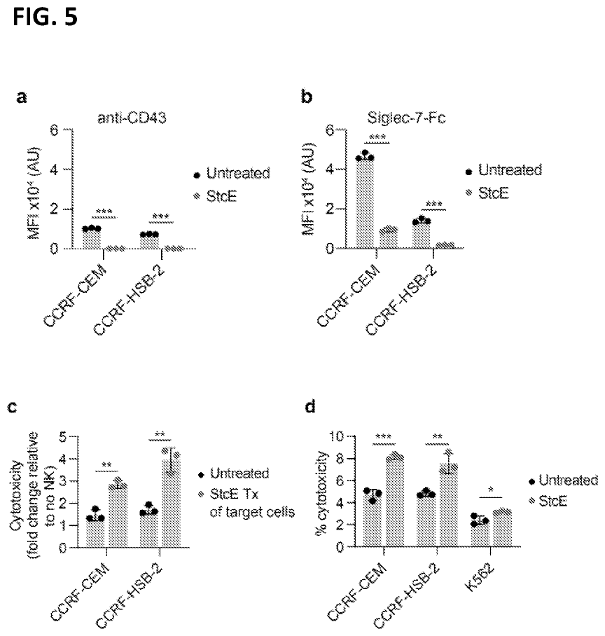

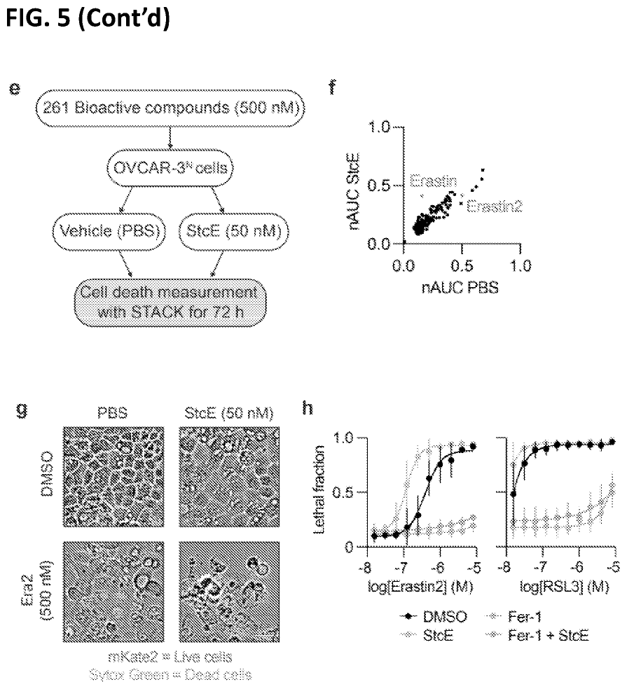

Next, tested was whether treatment with StcE could enhance immune surveillance of cancer cells. The mucin CD43 has been recently identified as a ligand on leukemia cells for the NK cell immune checkpoint receptor Siglec-717. In this model, removal of CD43 potentiates NK cell killing of leukemia cell lines. To assess whether mucinase treatment would have a similar effect, three leukemia cell lines were treated with or without endotoxin-free StcE (Methods), incubated them with healthy human blood donor NK cells, and quantified viability after 4 hours (FIG. 1d). StcE treatment resulted in loss of cell surface CD43 and overall Siglec-7 ligand residency, as expected (FIG. 1e and FIG. 5A-5B) 17. De-mucinated leukemia cells were susceptible to increased NK cell surveillance, while StcE treatment of NK cells had no effect relative to untreated control (FIG. 1f and FIG. 5c-d).

As the presence of mucins on the cell surface has been associated with decreased drug efficacy¹⁸, also explored was whether mucinase treatment would synergize with small molecule cytotoxic drugs. Using scalable time-lapse analysis of cell death kinetics (STACK) 19 (FIG. 5e), a 261 compound library was screened in the ovarian cancer cell line OVCAR-3. The commercial library of 261 small molecules targeted a range of biological pathways and had been used previously to evaluate treatments that modulate compound cytotoxicity⁵⁰. Erastin, which induces ferroptosis through inhibition of the cystine: glutamate antiporter system xc,²⁰ scored among the top hits for compound cell death enhanced by StcE treatment (FIG. 5f-g). A dose response with erastin², a more potent analog, confirmed enhancement of ferroptosis with StcE co-treatment that was fully suppressed by the ferroptosis inhibitor, ferrostatin-1. In contrast, there was no enhancement of ferroptosis induced by the mechanistically distinct compound RSL321 (FIG. 5h). Taken together, these results demonstrate that removal of mucins via mucinase treatment can reverse their pleotropic tumor-progressive roles.

### Example 2-Toxicity Profile of StcE Necessitates Tumor-Targeting

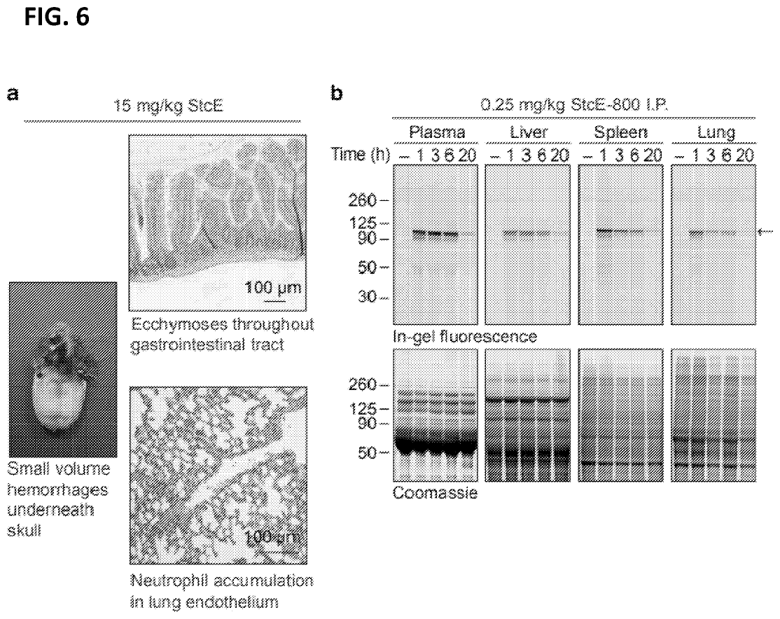

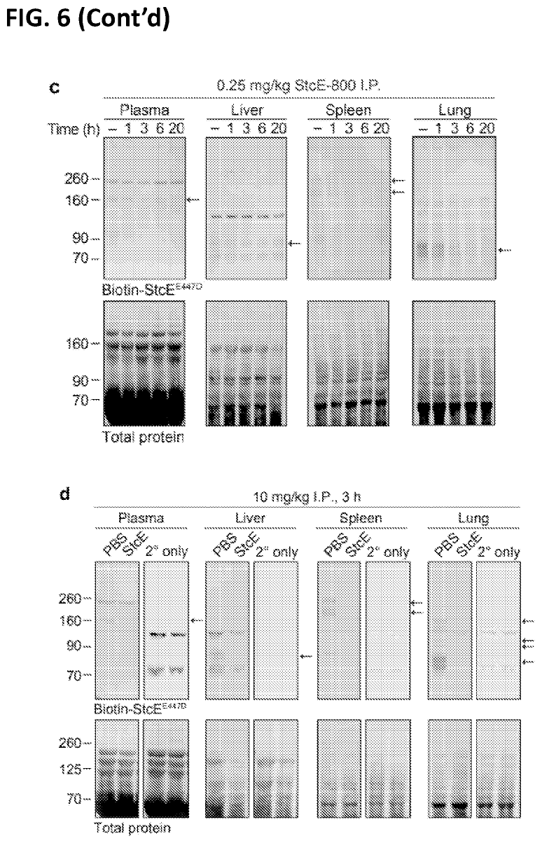

Bacterial enzymes are currently employed as frontline cancer therapeutics; for example, L-asparaginase from E. coli is used in childhood acute lymphoblastic leukemias²². As mucinases had not been tested as injectable therapeutics, StcE was assayed for activity and tolerability in vivo. The maximum tolerated dose for StcE treatment in Balb/c and C57BL/6 mice was 0.25 mg/kg. Necropsy and complete blood count (CBC) analyses performed 3 hours post injection of 15 mg/kg StcE revealed hemorrhages underneath the skull, ecchymoses throughout the gastrointestinal tract, neutrophil accumulation in the lungs, and platelet depletion (FIG. 6a). Western blot using a mucin-specific probe¹³ showed that StcE injected at 0.25 mg/kg remained in circulation for at least 20 hours and digested mucins throughout the body, though not as completely as higher doses (FIG. 6b-d and Table 3). As endothelial and white blood cell surface mucins are critical components of clotting and immune activation pathways, these findings established that an engineered mucinase variant with selectively for tumor mucins was necessary to avoid on target, off-tumor effects.

The clinical success of antibody-drug conjugates has shown that fusion of toxic therapeutic cargo to antibodies is a viable strategy for lowering on target, off-tumor toxicity and increasing on-target efficacy²³. More recently, antibody-enzyme conjugates have been designed to target the hydrolytic activity of an enzyme to specific subsets of cells²⁴. An important design principle of antibody-enzyme conjugates is to ensure that the activity of the enzyme is sufficiently low such that hydrolysis only occurs when the enzyme is concentrated at its target via binding of the antibody. Specifically, in previous work with an antibody of nanomolar affinity, micromolar enzymatic activity was shown to be effective for cell surface targets²⁵. As StcE is active at single digit nanomolar concentrations, our initial aim was to engineer a mucinase which retained its peptide and glycan specificity but exhibited activity within the micromolar range.
I.V. injection, 3 h, C57BL/6, n=1

TABLE 3
Complete blood count (CBC) analyses post I.V. injection of vehicle control
(PBS), StcE at 0.15 mg/kg, or StcE at 15 mg/kg (n = 1 animal per group).

| | | 0.15 mg/kg | 15 mg/kg | Reference | |
|---|---|---|---|---|---|
| | Control | StcE | StcE | Ranges | |
| White blood cell (WBC) | 5.86 | 4.44 | 3.94 | 5.5-9.3 | K/μL |
| Red blood cell (RBC) | 10.85 | 9.78 | 13.11 | 7.7-8.8 | M/μL |
| Hemoglobin (HGB) | 15.8 | 14 | 18.3 | 13.7-16.4 | gm/dL |
| Hematocrit (HCT) | 50.3 | 45.5 | 58.9 | 39.0-47.0% | |
| Mean corpuscular volume (MCV) | 46.4 | 46.5 | 44.9 | 52.0-68.7 | fL |
| Mean corpuscular hemoglobin (MCH) | 14.6 | 14.3 | 14 | 18.4-19.6 | pg |
| MHC concentration (MCHC) | 31.4 | 30.8 | 31.1 | 34.0-36.0 | g/dL |
| Platelet count | 1241 | 1345 | 82 | 675-1338 | K/μL |

| | | | | |
|---|---|---|---|---|
| Red cell distribution width (RDW) | 20 | 19.2 | 22.2 | % |
| Platelet distribution width (PDW) | 7.8 | 8.9 | 7.2 | fL |
| Mean platelet volume (MPV) | 6.6 | 7.3 | 6.8 | fL |
| Platelet-large cell ratio (P-LCR) | 4.9 | 8.7 | 5.5 | % |
| Procalcitonin (PCT) | 0.82 | 0.98 | 0.06 | % |
| Reticulocyte count | 4.88 | 4.52 | 6.56 | 1.0-2.8% |
| Immature reticulocyte fraction (IRF) | 50.3 | 54.9 | 52.3 | % |
| Low fluorescence reticulocytes (LFR) | 49.7 | 45.1 | 47.7 | % |
| Medium fluorescence | 24.6 | 18.7 | 28 | % |
| reticulocytes (MFR) | | | | |
| Reticulocyte absolute | 529480 | 442056 | 860016 | /μL |
| Platelet estimate | Adequate | Adequate | Decreased | K/μL |
| Neutrophils | 4 | 4 | 54 | 15-32% |
| Lymphocytes | 93 | 92 | 37 | 65-83% |
| Monocytes | 2 | 3 | 8 | 0-3% |
| Eosinophils | 1 | 1 | 1 | 0-3% |
| Basophils | 0 | 0 | 0 | % |
| RBC morphology | Normal | Normal | Normal | |
| Neutrophil absolute | 234 | 178 | 2128 | 825-2604 |
| Lymphocyte absolute | 5450 | 4085 | 1458 | 3685-7812 |
| Monocyte absolute | 117 | 133 | 315 | 0-279 |
| Eosinophil absolute | 59 | 44 | 39 | 0-279 |
| Basophil absolute | 0 | 0 | 0 | N/A |

### Example 3-Structure-Guided Engineering Reduces Activity, Binding, and Size of StcE

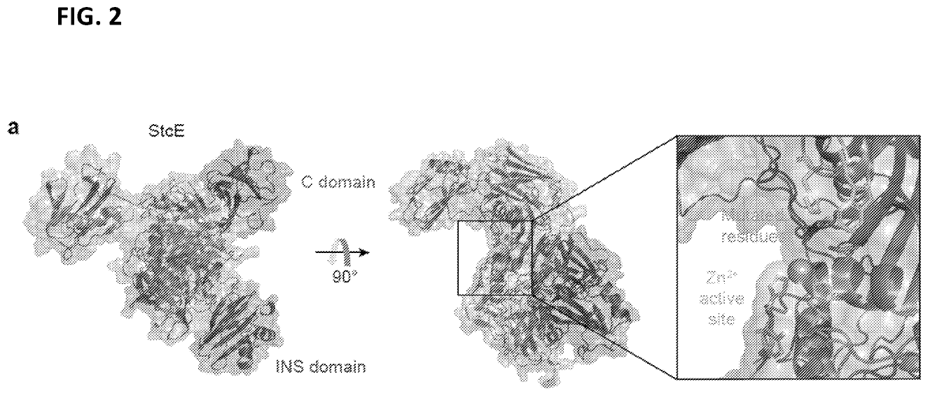

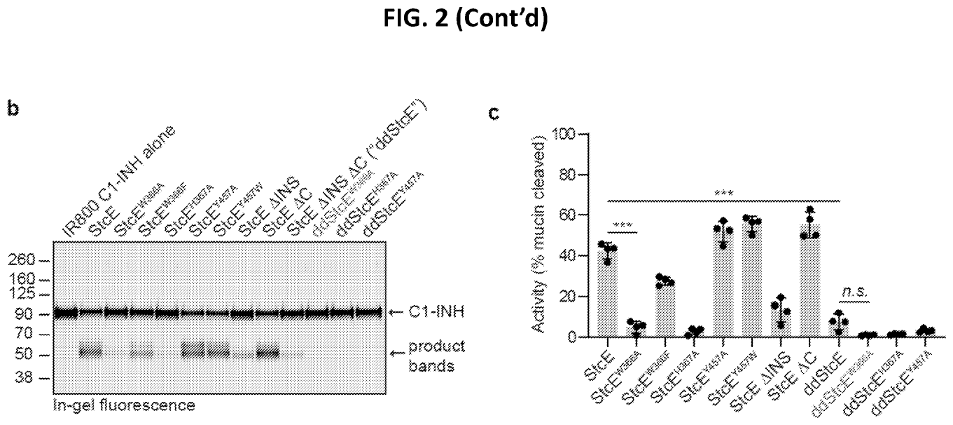

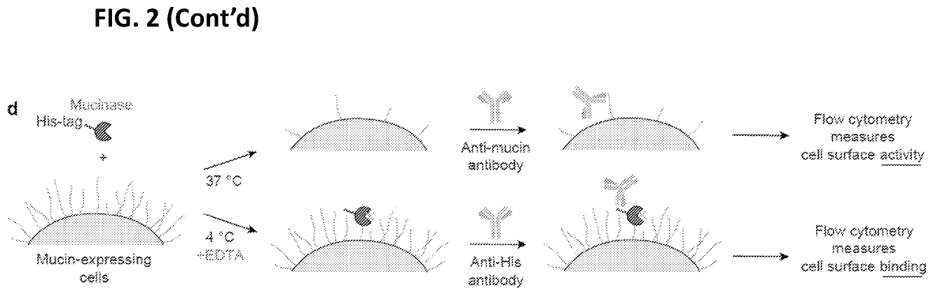

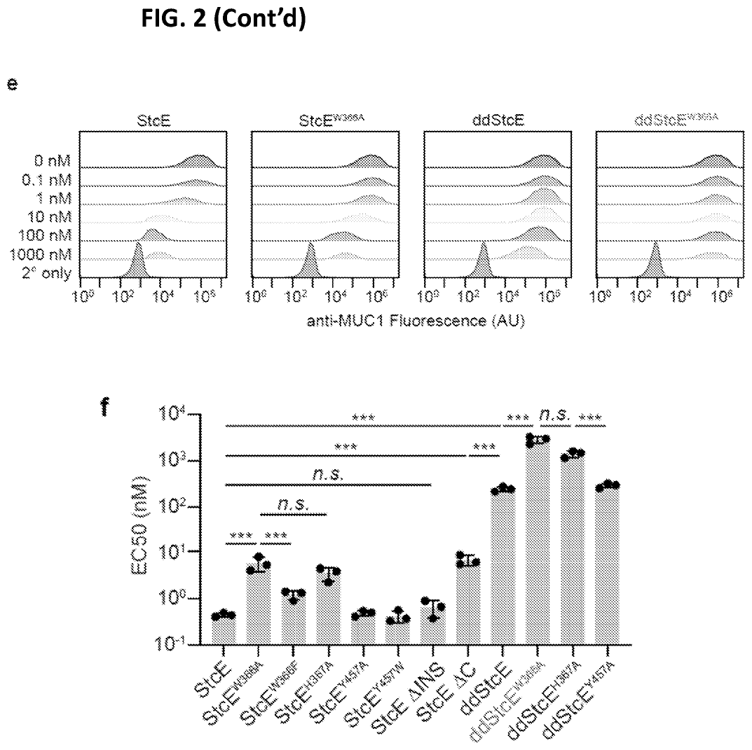

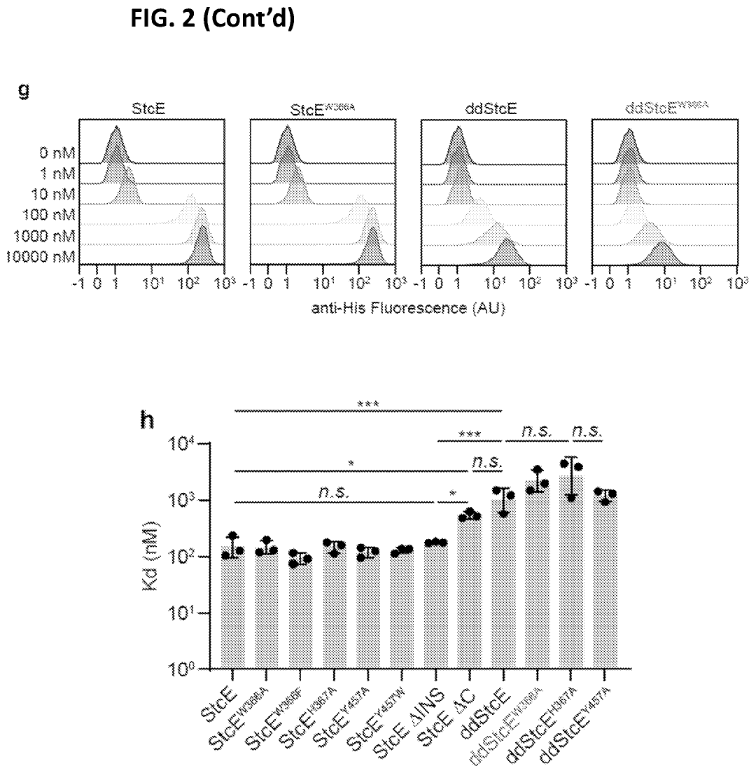

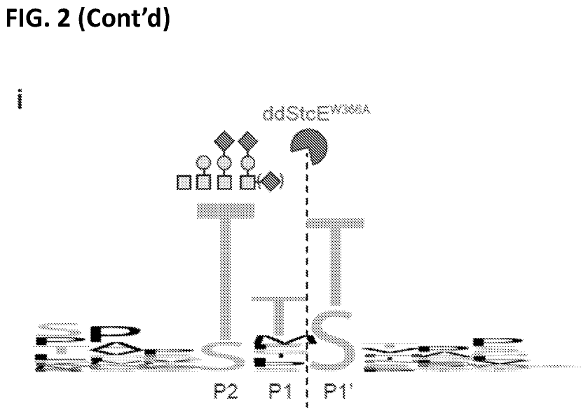

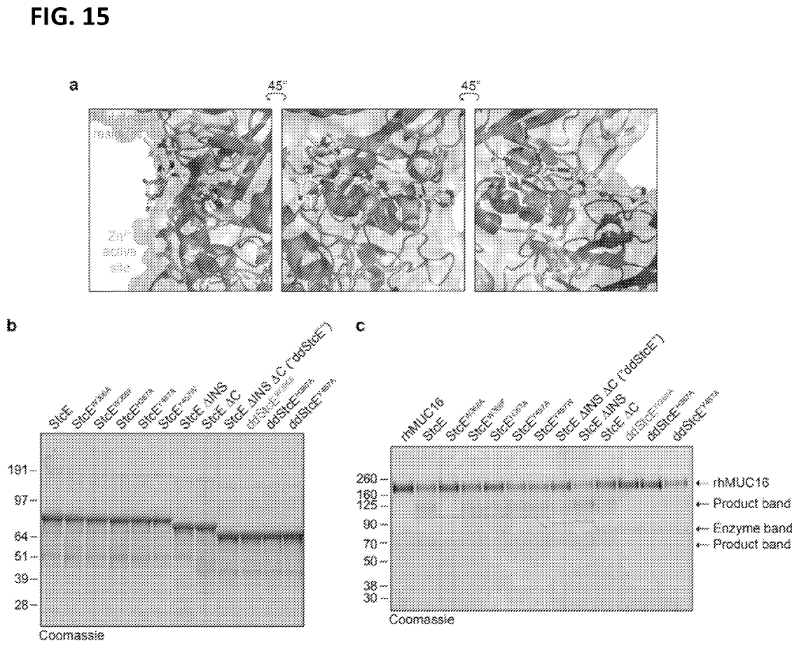

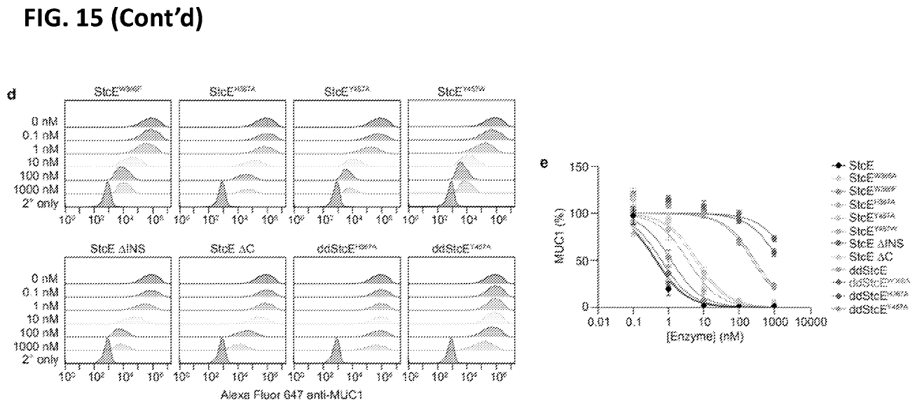

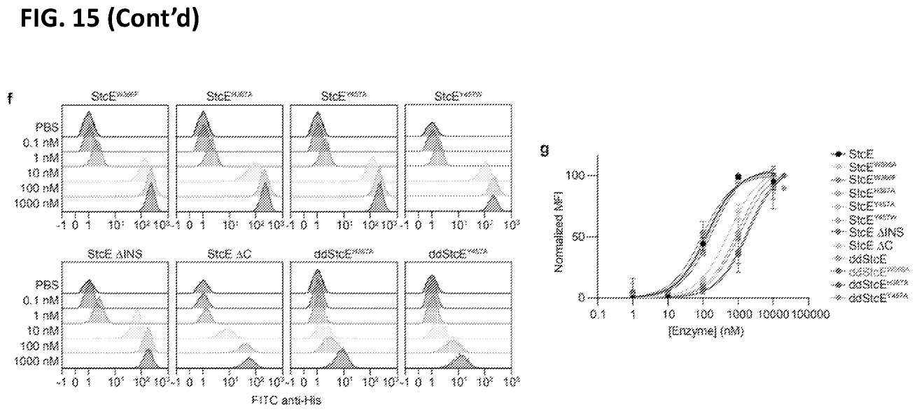

A previously published crystal structure²⁶ and prior docking studies¹² was utilized to rationally design a StcE mutant with reduced activity and cell surface binding but retained specificity for mucins. To begin, two domains were deleted, the C and INS domains (FIG. 2a, left), as removal of the INS domain had previously been observed to reduce enzymatic activity and removal of the C domain had been shown to decrease nonspecific cell surface binding²⁶, both of which were desirable for our targeted mucinase. Also mutated were amino acids found near the active site that were not predicted to interact with key substrate residues in the docked complex. Specifically, Trp366, His367, and Tyr457 line the active site but do not directly interact with (i) enzyme catalytic residues or (ii) substrate P2-P1′ residues, which comprise the S/T*-X-S/T cleavage motif of StcE, suggesting that these residues could be candidates for mutation aimed at reducing but not abrogating enzymatic activity (FIG. 2a, right and FIG. 15a)

Combinations of domain deletions and single point mutations yielded a total of eleven mutants for characterization, with expression yields ranging from 20-100 mg/L (Methods and FIG. 15B). In vitro activity against recombinant mucin substrates was quantified by densitometry following SDS-PAGE (FIG. 2b-c and FIG. 15C). To determine the half maximal effective concentration (EC50) of the mutants for degradation of mucins on live cell surfaces, cells were treated with various concentrations of enzymes for 1 hour at 37° C. and subjected to flow cytometry for cell surface MUC1 (FIG. 2d-f and FIG. 15d-e). To determine effective dissociation constants (Kd) of the mutants, cells were treated with the same concentrations of enzymes for 30 mins at 4° C. in the presence of the metal chelator and metalloprotease inhibitor EDTA, to prevent enzyme activity. In the latter case, binding was quantified via interaction of an anti-His tag antibody with the His-tagged mutants (FIG. 2d,g-h and FIG. 15f-g).

Combining the two domain deletions (ΔC and ΔINS) yielded ddStcE (double deletion StcE), which exhibited reduced activity and binding, along with a molecular weight reduction to 76 kDa relative to the 98 kDa parent enzyme. Nevertheless, ddStcE's EC50 and Kd on cells remained in the high nanomolar range. (for discussion of enzymatic activity relative to binder affinity, see above). Of the single point mutations, W336A and H367A most drastically reduced activity against recombinant and cell surface mucins (FIG. 2c,f). Adding W336A and H367A mutations to the ddStcE scaffold yielded ddStcE^{W366A} and ddStcE^{H367A}, which were active in the desired micromolar range, with approximate EC50 values of ˜3 and ˜1 μM, respectively, and effective Kd values of ˜2 μM each (FIG. 2f,h). ddStcE^{W366A}, referred to herein as engineered StcE or “eStcE”, was selected as the scaffold for the targeted enzyme, because it exhibited lowest activity against cell surface MUC1. The cleavage motif of eStcE was characterized using mass spectrometry as was previously done for StcE²⁷, revealing that the mutations did not alter substrate recognition (FIG. 2i).

### Example 4-a Nanobody-eStcE Conjugate Achieves Targeted Mucin Degradation

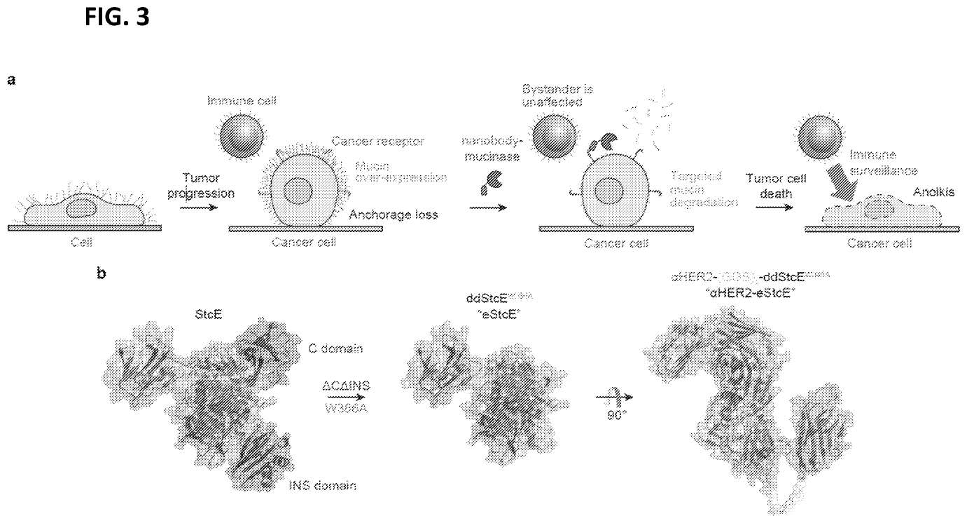

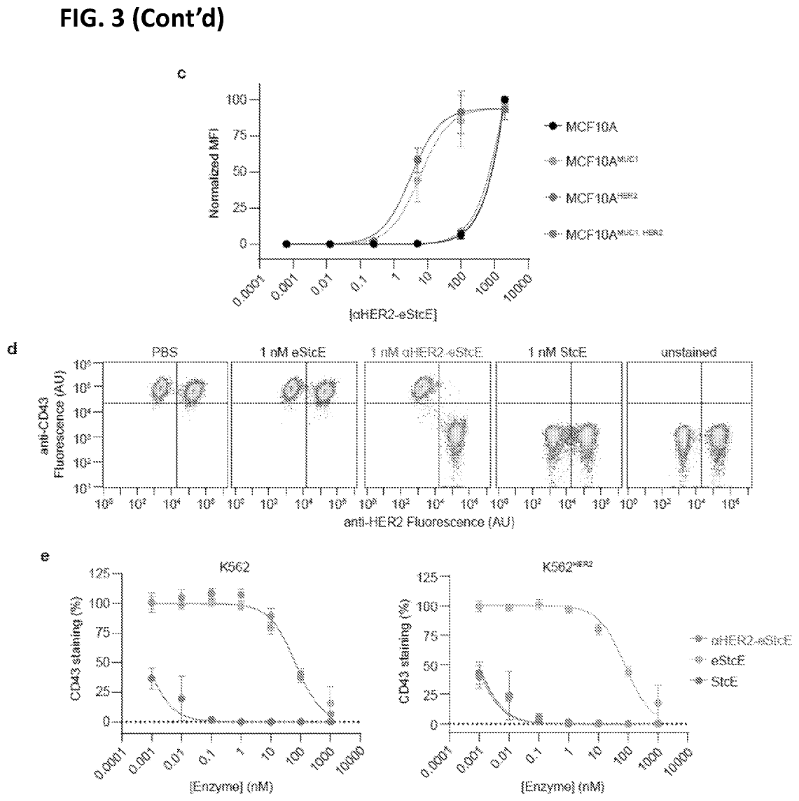

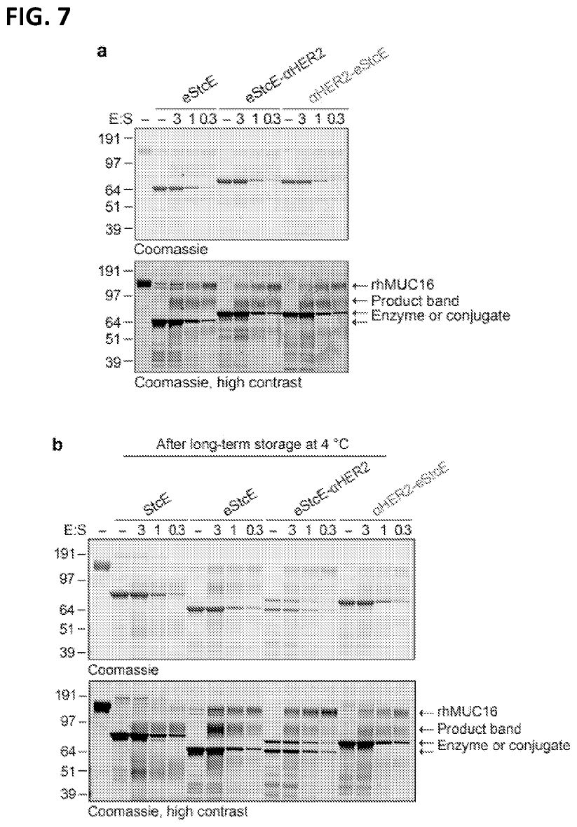

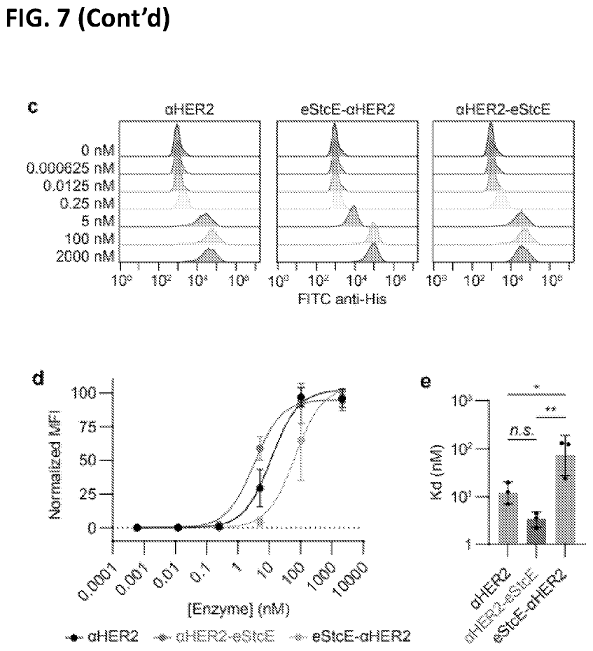

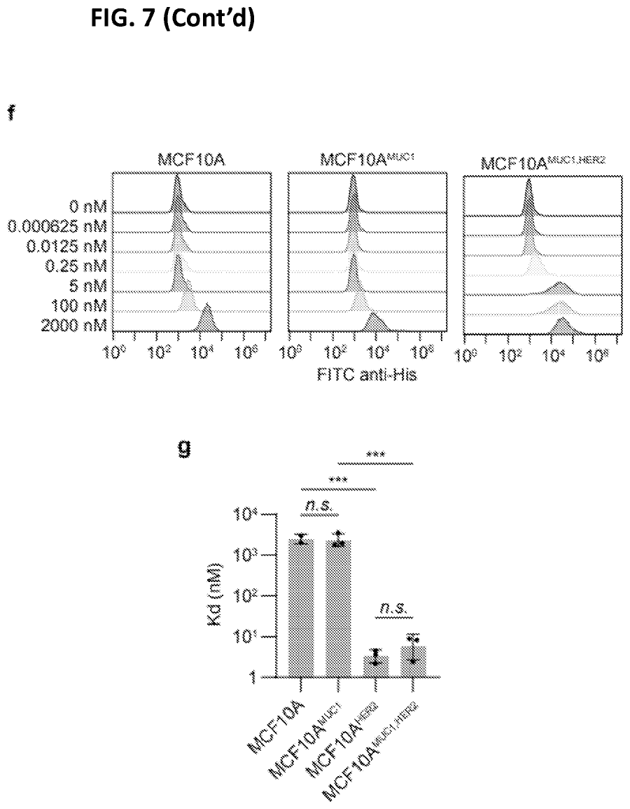

It was next envisioned that targeting eStcE to cancer cells would reverse biophysical and immunological tumor-progressive pathways while leaving bystander cells unaffected (FIG. 3a). As immune cells bind Fc-containing biologics through the Fc receptor²⁸, a genetic fusion to a nanobody was created rather than an antibody. The cell surface receptor HER2 was selected as the target antigen because it is upregulated in several carcinoma subtypes, including breast and ovarian, and is bound by a well validated nanobody, 5F7²⁹. Two different fusion orientations were designed and tested for expression yield, stability, mucinase activity, and cell surface HER2 binding (FIG. 3b). Both orientations of the chimera expressed in endotoxin-deficient ClearColi at 30-60 mg/L and were similarly active against recombinant MUC16 as eStcE alone (FIG. 7a). The conjugate with the mucinase C-terminal to the nanobody, referred to herein as “αHER2-eStcE”, was stable and retained activity after months at 4° C., while the other orientation “eStcE-αHER2” exhibited reduced activity following equivalent storage conditions (FIG. 7b). Effective dissociation constants (Kd) for nanobody-mucinase conjugates were determined via flow cytometry of HER2⁺ cells as described above, giving values of 11, 4, and 58 nM for αHER2, αHER2-eStcE, and eStcE-αHER2, respectively (FIG. 7c-e). Therefore, αHER2-eStcE was selected for further in cellulo and in vivo analyses due to its increased stability and binding to HER2⁺ cells.

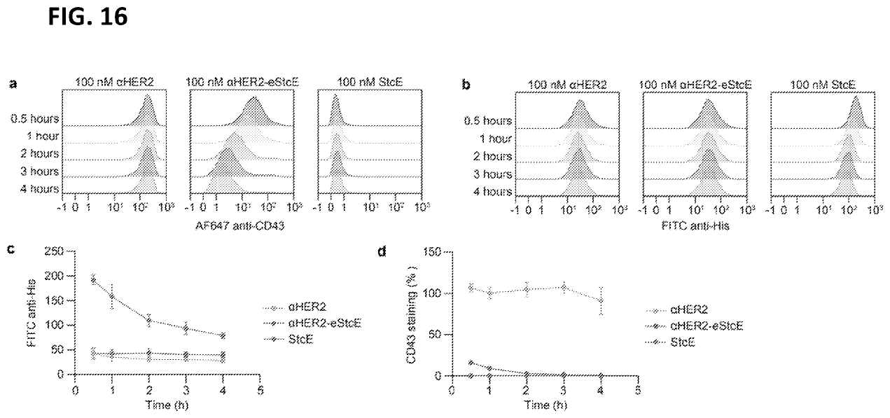

Cells transduced with a doxycycline-inducible MUC1 ectodomain construct were used to test the contributions of the nanobody and the enzyme components of αHER2-eStcE to mucin binding. αHER2-eStcE bound to HER2⁺ cell surfaces with a Kd value approximately three orders of magnitude higher relative to its binding to HER2-cells, indicating that αHER2-eStcE bound to cells via HER2 affinity and not mucin affinity (FIG. 3c and FIG. 7f-g). Indeed, HER2⁺ cells treated with αHER2-eStcE over a 4-hour time course exhibited an approximately 10-fold decrease in CD43 staining but did not display loss in αHER2-eStcE cell surface residency, indicating that the conjugate does not need to bind mucins stably in order to deplete cellular mucins (FIG. 16).

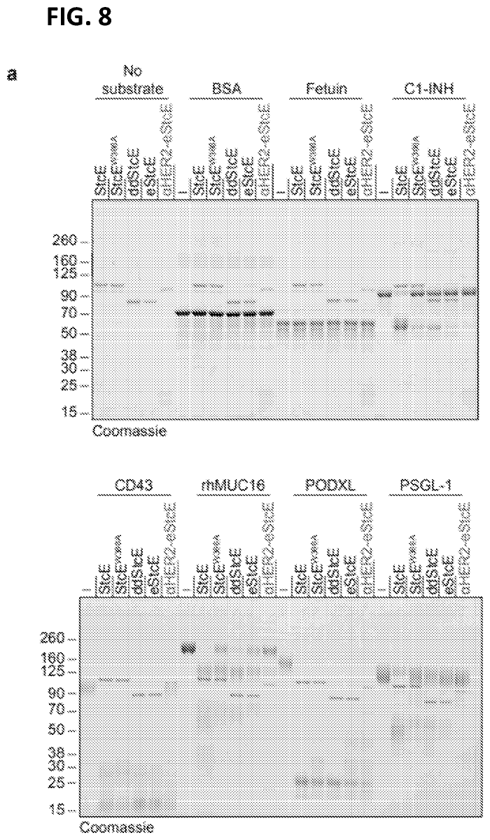

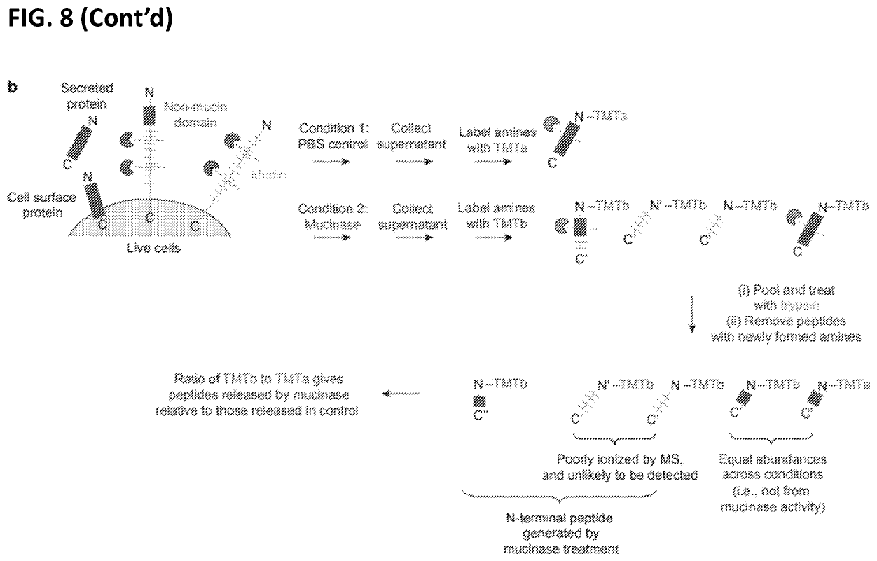

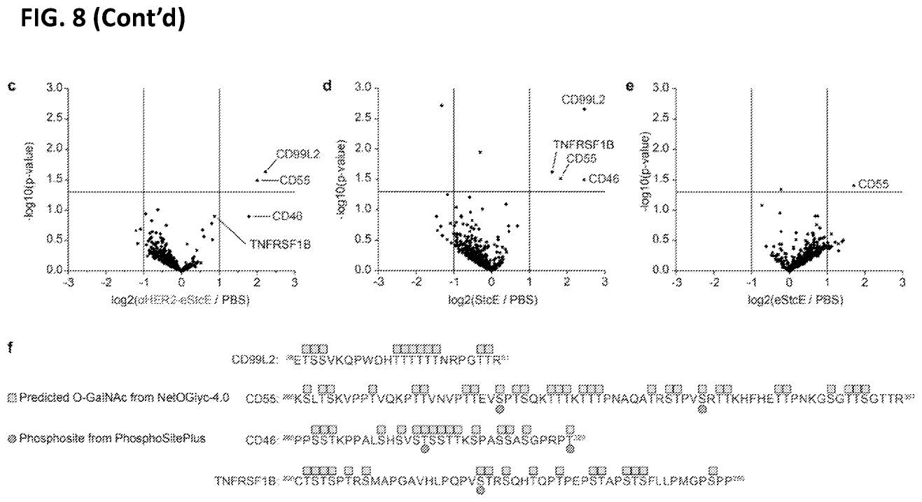

Cleavage assays with a panel of recombinant non-mucin and mucin proteins confirmed that the conjugates maintained mucin selectivity (FIG. 8a). In order to test the specificity of αHER2-eStcE for mucins versus non-mucin proteins on cell surfaces, terminal amine isotopic labeling of substrates (TAILS) mass spectrometry was employed, which is a method optimized for detection of novel peptides generated from protease digestion of live cells³⁰. A HER2+ suspension cell line was treated with vehicle control, StcE, eStcE, or αHER2-eStcE, and supernatants were collected and subjected to TAILS MS (FIG. 8b). Importantly, analysis of peptides generated relative to vehicle control confirmed selectivity for mucin peptides, similar cleavage profiles between StcE and αHER2-eStcE, and reduced activity for untargeted eStcE (FIG. 8c-f).

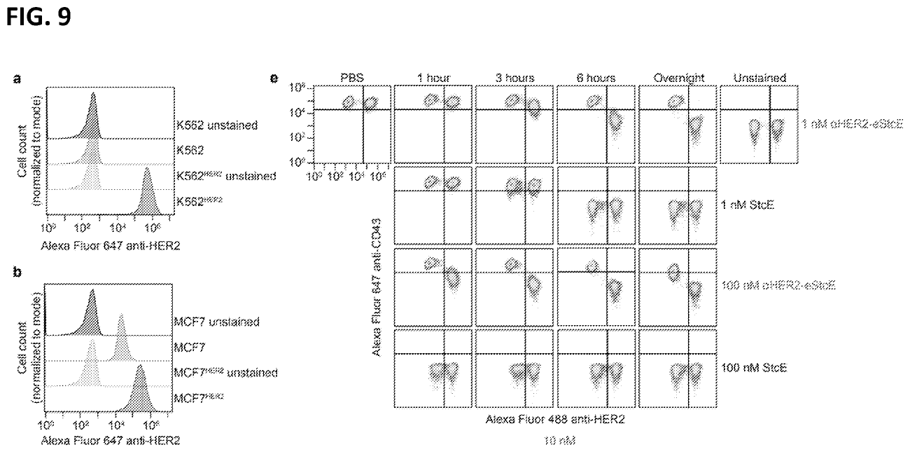

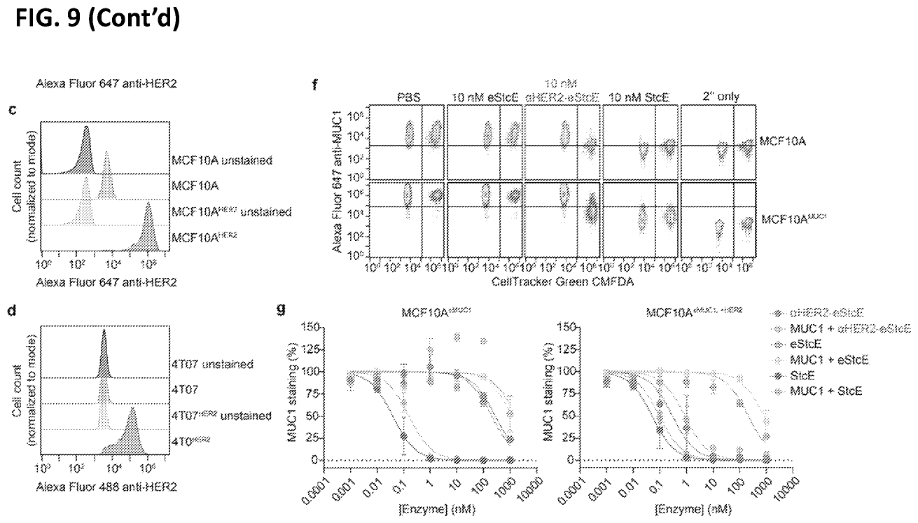

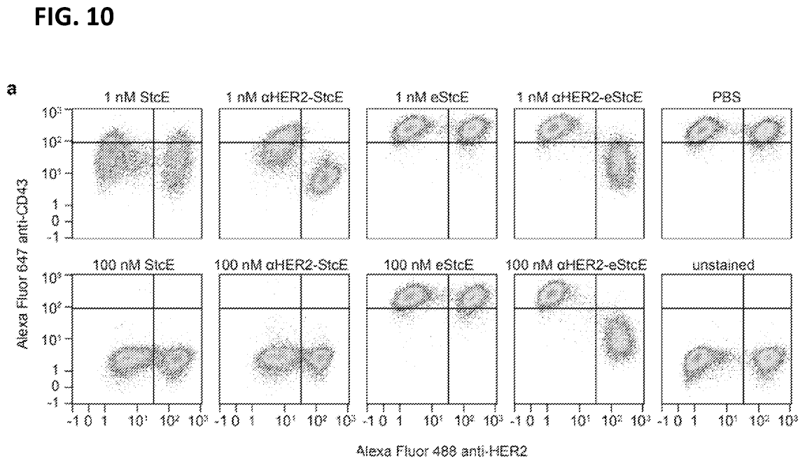

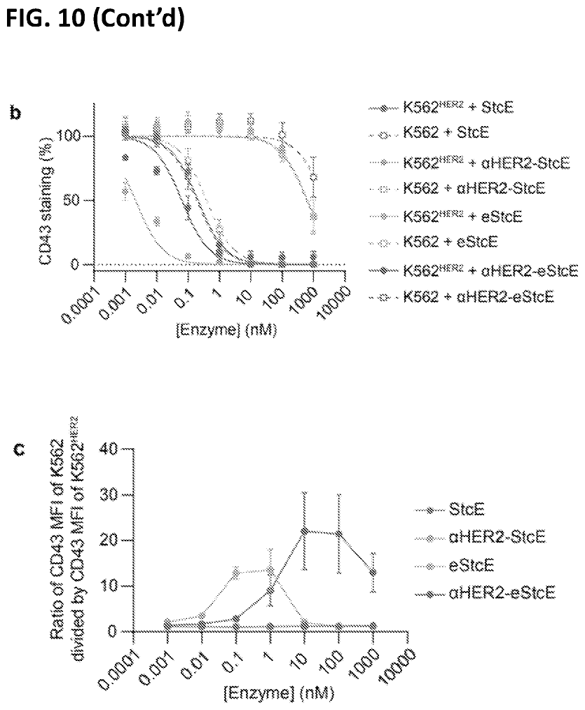

Next, the conjugate's on-target activity was tested through mixed cell assays with various human cancer cell lines engineered to express HER2 (FIG. 9a-d). Mixed HER2⁺ and HER2-cells were treated with StcE, eStcE, or αHER2-eStcE overnight, and depletion of cell surface mucins was analyzed via live cell flow cytometry. StcE treatment at 1 nM resulted in complete removal of cell surface mucins on both HER2⁺ and HER2-cells, while 1 nM of eStcE resulted in no discernable removal of mucins in either population. In contrast, 1 nM of αHER2-eStcE resulted in complete loss of cell surface mucins on HER2⁺ cells and no discernable loss of mucins on HER2-cells (FIG. 3d, quantified in FIG. 3e, time course in FIG. 9e). The same trend was observed at higher doses in another cell line interrogated for cell surface residency of a different mucin protein (FIG. 9f-g). A fusion of the parent enzyme StcE to the nanobody, “αHER2-StcE”, was unable to remove mucins solely on HER2⁺ cells at any tested concentration (0.001 to 1000 nM), confirming the need for engineering of a lower activity mutant (FIG. 10).

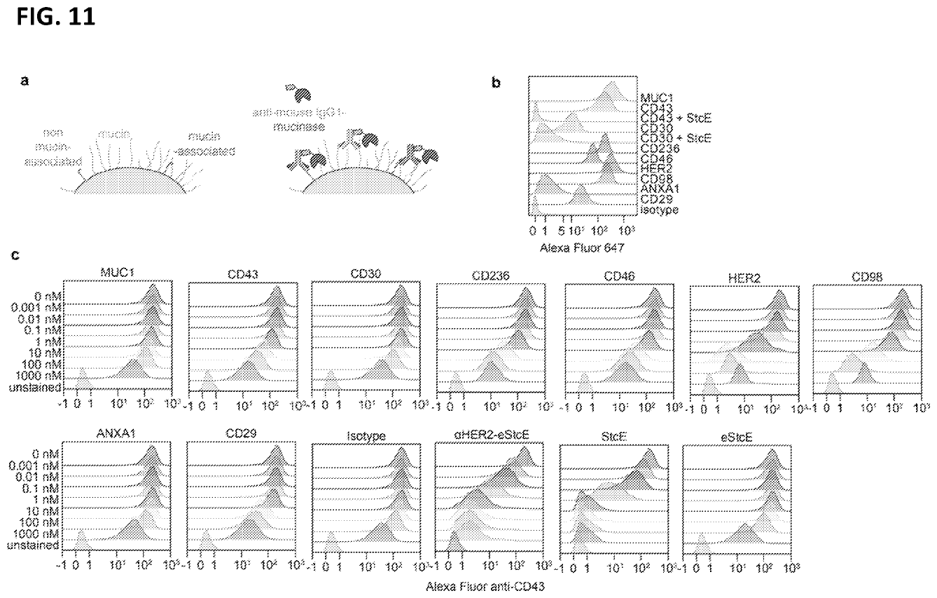

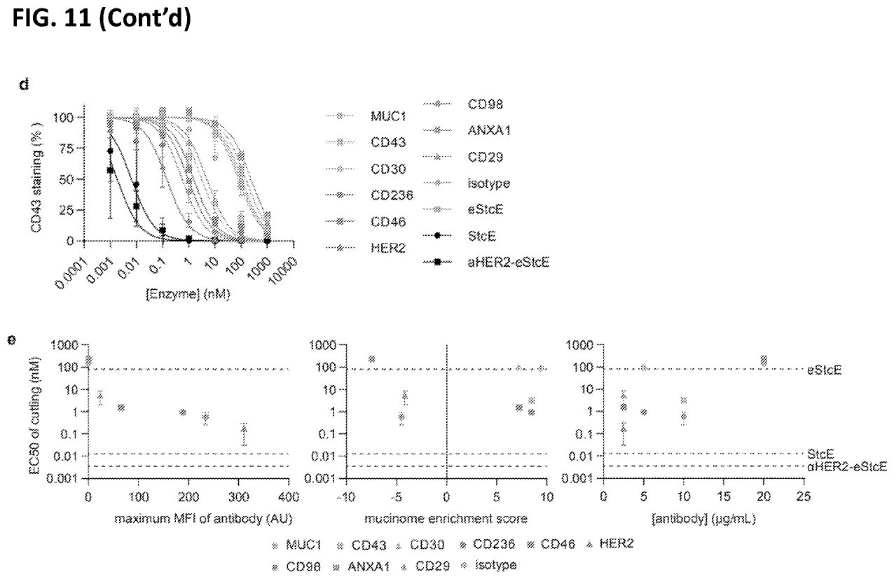

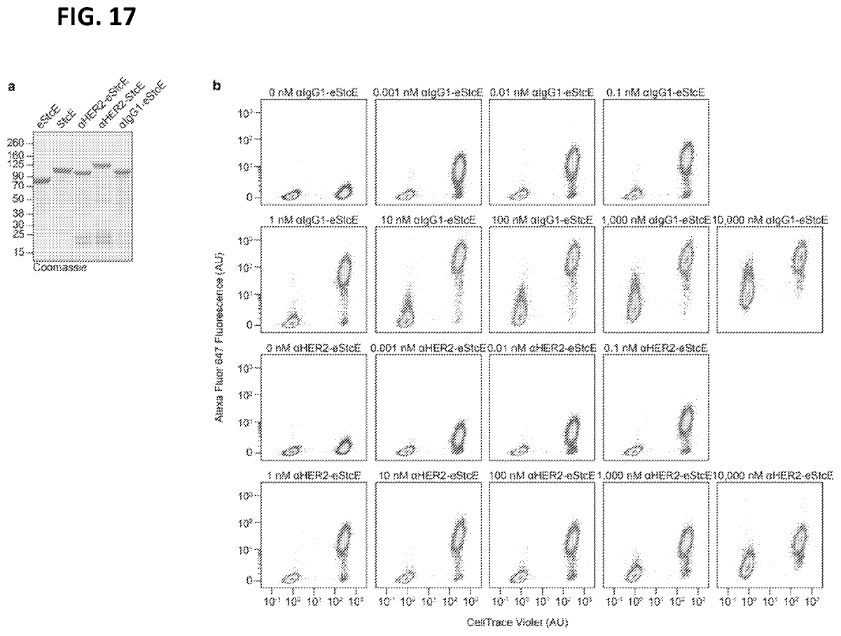

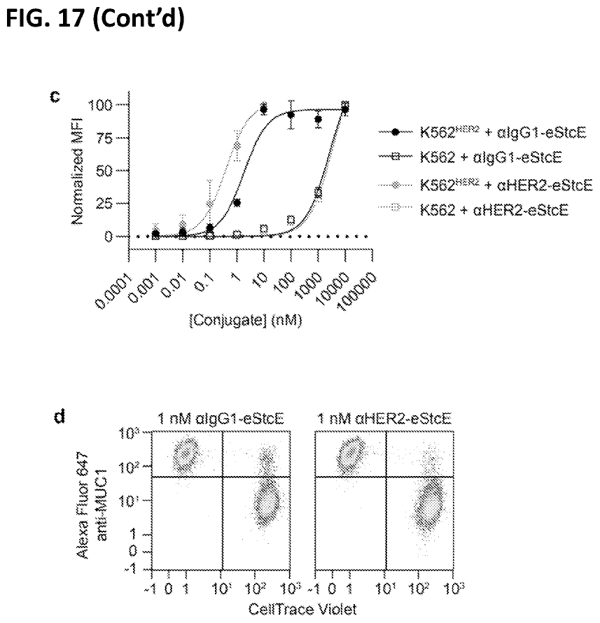

Finally, to address the generalizability of the targeted mucin degradation approach, a previously published and well-validated anti-mouse IgG1 Fc nanobody (TP1107)⁵¹ was fused to eStcE to generate a conjugate termed “αIgG1-eStcE”, and it was confirmed that it performed comparably to αHER2-eStcE (FIG. 11 and FIG. 17). Cells were co-treated with αIgG1-eStcE and primary antibodies against mucin, mucin-associated, and non-mucin associated cell surface antigens, chosen based on enrichment scores from the recently published cell mucinome (Methods). The resulting EC50s for mucin depletion were plotted against the concentration of primary antibody used, the target's mucinome enrichment score, and the maximum median fluorescence intensity (MFI) of primary binding. The MFI of primary antibody binding was the sole factor that correlated with EC50 trends, suggesting the absence of any specific requirement for the targeting agent beyond the ability to load enzyme onto cell surfaces.

The amino acid sequence of αHER2-eStcE is provided in Table 2 below. An N-terminal His tag and GGS linker are shown in italics. The αHER2 nanobody is indicated by bold. ddStcE^{W366A} is underlined.

TABLE 2
αHER2-eStcE Amino Acid Sequence

| Description | Amino Acid Sequence | SEQ ID NO: |
|---|---|---|
| αHER2-eStcE | MGSSHHHHHHSSGLVPRGSHMASEVQLVESGGGLVQA | 15 |
| | GGSLRLSCAASGITFSINTMGWYRQAPGKQRELVALISSI | |
| | GDTYYADSVKGRFTISRDNAKNTVYLQMNSLKPEDTAVY | |
| | YCKRFRTAAQGTDYWGQGTQVTVSSGGSGGSGGSGGS | |
| | ADNNSAIYFNTSQPINDLQGSLAAEVKFAQSQILPAHPKEG | |
| | DSQPHLTSLRKSLLLVRPVKADDKTPVQVEARDDNNKILG | |
| | TLTLYPPSSLPDTIYHLDGVPEGRSGELENNRITYAQHIWS | |
| | AELPAHWIVPGLNLVIKQGNLSGRLNDIKIGAPGELLLHTIDI | |
| | GMLTTPRDRFDFAKDKEAHREYFQTIPVSRMIVNNYAPLH | |
| | LKEVMLPTGELLTDMDPGNGGAHSGTMRQRIGKELVSHG | |
| | IDNANYGLNSTAGLGENSHPYVVAQLAAHNSRGNYANGI | |
| | QVHGGSGGGGIVTLDSTLGNEFSHEVGHNYGLGHYVDG | |
| | FKGSVHRSAENNNSTWGWDGDKKRFIPNFYPSQTNEKS | |
| | CLNNQCQEPFDGHKFGFDAMAGGSPFSAANRFTMYTPN | |
| | SSAIIQRFFENKAVFDSRSSTGFSKWNADTQEMEPYEHTI | |
| | DRAEQITASVNELSESKMAELMAEYAVVKVHMWNGNWT | |
| | RNIYIPTASADNRGSILTINHEAGYNSYLFINGDEKVVSQGY | |
| | KKSFVSDGQFWKERDVVDTREARKPEQFGVPVTTLVGYY | |
| | DPEGTLSSYIYPAMYGAYGFTYSDDSQNLSDNDCQLQVD | |
| | TKEGQLRFRLANHRANNTVMNKFHINVPTESQPTQATLVC | |
| | NNKILDTKSLTPAPEGLTYTVNGQALPAK | |

### Example 5-αHER2-eStcE Selectively Kills HER2⁺ Cells in Mixed Cell Assays and is Nontoxic in Mice

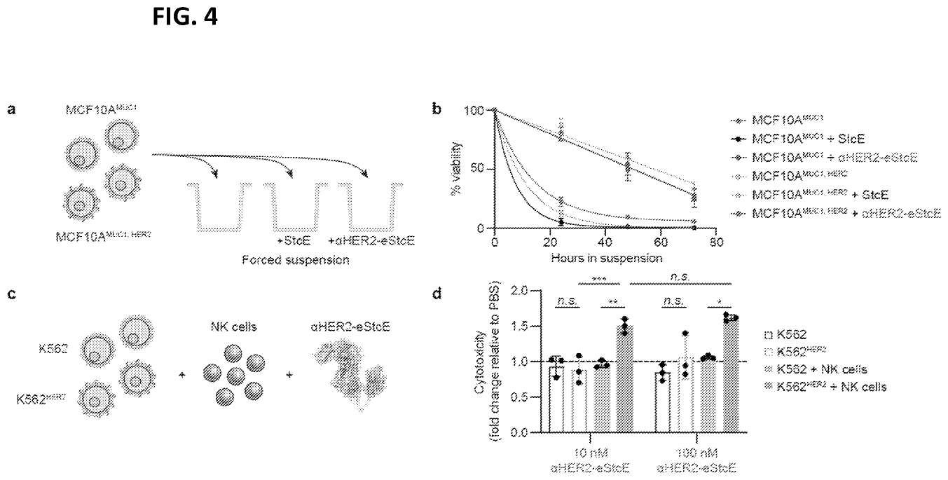

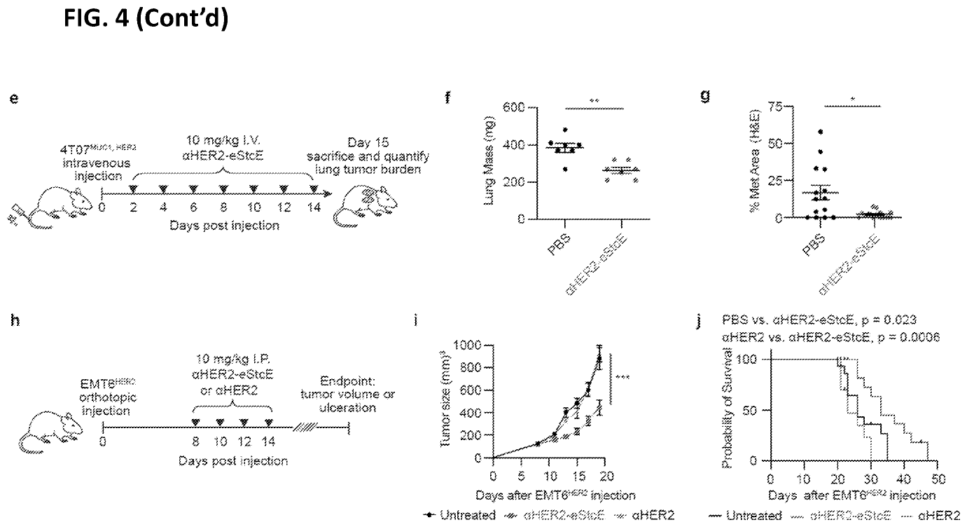

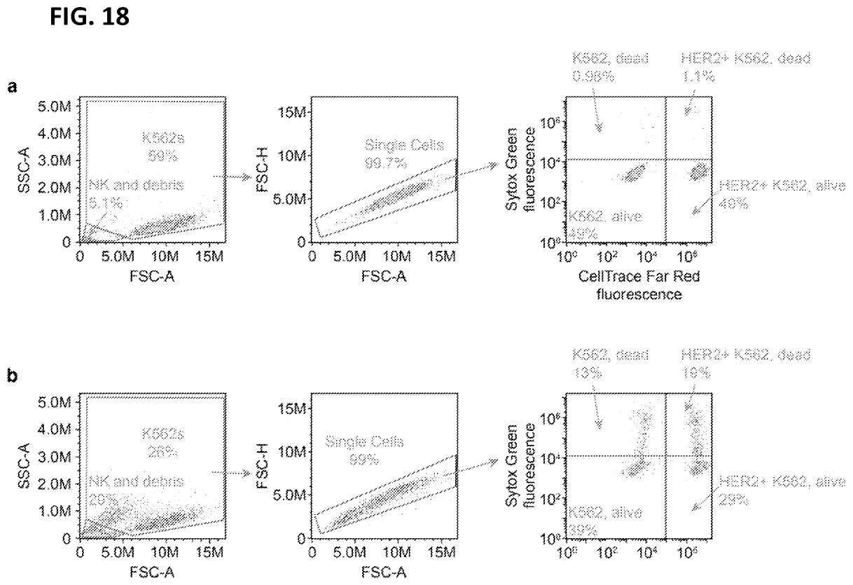

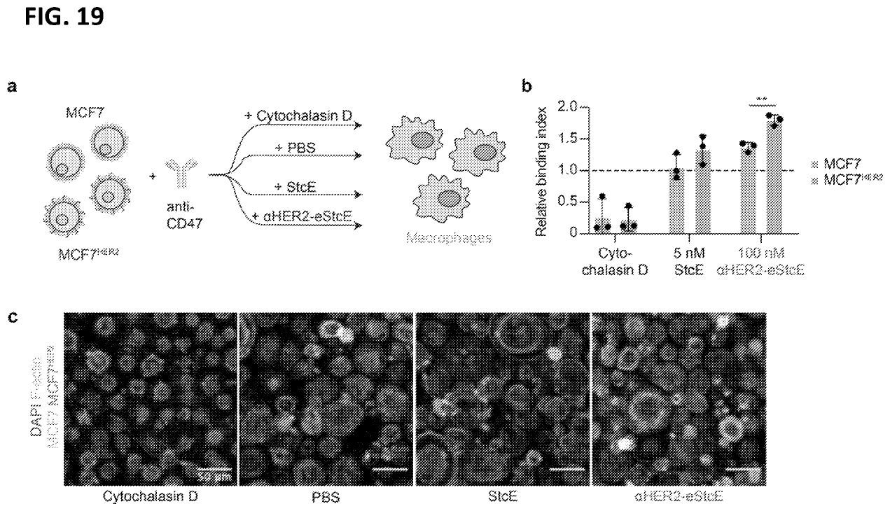

Tested using a series of mixed cell assays was whether αHER2-eStcE could selectively reverse mucin-dependent tumor-progressive pathways. Biophysical and immunological assays from FIG. 1 were repeated using HER2⁺ and HER2-cell populations, which were mixed prior to enzymatic treatment (FIG. 4a,c). Under anchorage-free conditions, αHER2-eStcE reversed suspension survival in only the HER2⁺ population whereas StcE treatment resulted in cell death in both populations (FIG. 4b). Likewise, treatment with αHER2-eStcE selectively enhanced NK cell-mediated killing of the HER2⁺ population (FIG. 4d and FIG. 18). Recently, it was found that removal of cell surface mucins via StcE treatment promotes engagement of trans-acting phagocytic receptors in macrophages³¹. Thus, αHER2-eStcE was tested in a mixed cell assay with primary macrophages, where enhancement of phagocytosis of HER2⁺ cells over HER2-cells was observed (FIG. 19).

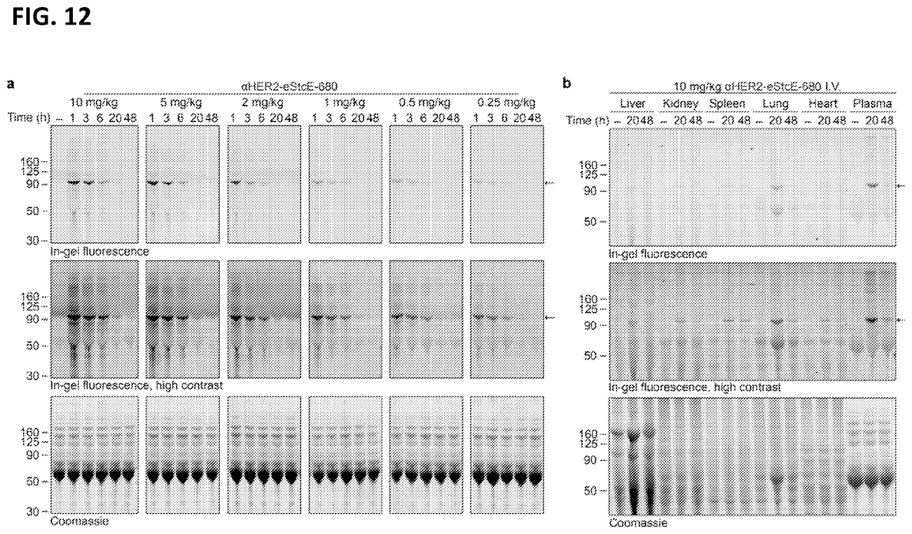

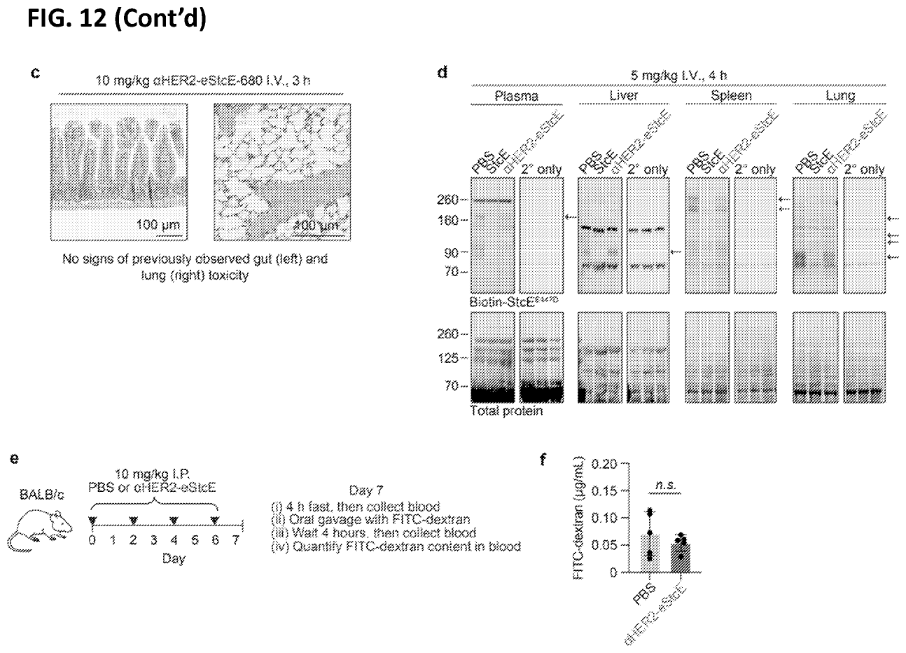

Intravenous administration of fluorophore-labeled αHER2-eStcE at doses ranging from 0.25-10 mg/kg into Balb/c mice revealed that the conjugate remained in blood and tissues for approximately at least 20 hours, with no discernable toxicity (FIG. 12a-12b). Blinded necropsy and complete blood count (CBC) analyses confirmed no abnormalities at the highest tested dose of 10 mg/kg (FIG. 12c and Table 4). To assess mucin depletion in tissues, 5 mg/kg StcE or αHER2-eStcE was intravenously injected into Balb/c mice, followed by collection of plasma, liver, spleen, and lung 4 hours post injection, and immunoblotting for mucins. In all cases, αHER2-eStcE injection resulted in significantly reduced mucin depletion when compared to the wild-type parent enzyme (FIG. 12d). The integrity of the gastrointestinal mucus layer was also maintained with repeated doses (FIG. 12e-f).
I.P. injection, 3 h, BALB/cJ, average of n=2

TABLE 4
Complete blood count (CBC) analyses post I.P. injection
of vehicle control (PBS) or αHER2-eStcE at 10
mg/kg (average of n = 2 animals per group).

| | | 10 mg/kg | | |
|---|---|---|---|---|
| | | αHER2- | Reference | |
| | Control | eStcE | Ranges | |
| White blood cell | 5.55 | 5.6 | 5.5-9.3 | K/μL |
| (WBC) | | | | |
| Red blood cell (RBC) | 11.23 | 10.42 | 7.7-8.8 | M/μL |
| Hemoglobin (HGB) | 17.3 | 15.95 | 13.7-16.4 | gm/dL |
| Hematocrit (HCT) | 51.85 | 48.2 | 39.0-47.0% | |
| Mean corpuscular | 46.2 | 46.25 | 52.0-68.7 | fL |
| volume (MCV) | | | | |
| Mean corpuscular | 15.4 | 15.3 | 18.4-19.6 | pg |
| hemoglobin (MCH) | | | | |
| MHC concentration | 33.35 | 33.1 | 34.0-36.0 | g/dL |
| (MCHC) | | | | |
| Platelet count | 1121 | 1030 | 675-1338 | K/μL |

| | | | |
|---|---|---|---|
| Red cell distribution | 21.45 | 20.45 | % |
| width (RDW) | | | |
| Platelet distribution | 13.2 | 13.4 | fL |
| width (PDW) | | | |
| Mean platelet volume | 8.5 | 8.6 | fL |
| (MPV) | | | |
| Platelet-large cell | 19.85 | 20.15 | % |
| ratio (P-LCR) | | | |
| Procalcitonin (PCT) | 0.96 | 0.88 | % |
| Reticulocyte count | 4.16 | 3.95 | 1.0-2.8% |
| Immature reticulocyte | 46.26 | 47.9 | % |
| fraction (IRF) | | | |
| Low fluorescence | 53.75 | 52.1 | % |
| reticulocytes (LFR) | | | |
| Medium fluorescence | 16.95 | 17.15 | % |
| reticulocytes (MFR) | | | |
| Reticulocyte absolute | 466118 | 410464 | /μL |
| Platelet estimate | Adequate | Adequate | K/μL |
| Neutrophils | 14 | 33 | 15-32% |
| Lymphocytes | 82 | 54 | 65-83% |
| Monocytes | 3.5 | 13.5 | 0-3% |
| Eosinophils | 1 | 0 | 0-3% |
| Basophils | 0 | 0 | % |
| RBC morphology | Normal | Normal | |
| Neutrophil absolute | 767 | 1835 | 825-2604 |
| Lymphocyte absolute | 4533 | 3006 | 3685-7812 |
| Monocyte absolute | 197 | 760 | 0-279 |
| Eosinophil absolute | 59 | 0 | 0-279 |
| Basophil absolute | 0 | 0 | N/A |

Note, apparent monocytosis in control and treated animals, when taken together with the presence of lymphopenia and the fact that animals were injected in the abdomen 3 hours prior, likely represents a stress response.

### Example 6-αHER2-eStcE Blunted Tumor Progression in Two Models of Murine Breast Cancer

To assess on-target efficacy of αHER2-eStcE, previously validated murine models of breast cancer progression were employed. The murine cell line 4T07 is a Balb/c syngeneic mammary carcinoma that efficiently metastasizes to sites such as the lung, but is unable to efficiently proliferate at metastatic sites³². Woods et al. showed that elaboration of 4T07 cell surfaces via ectopic expression of MUC1 ectodomain or with lipid-anchored mucin mimetic glycopolymers enhances proliferation in the metastatic niche through PI3K-Akt mechanosignaling pathways related to cell cycle progression³³. This model involved tail vein injection of luciferase-expressing 4T07 cells into Balb/c mice, whereupon cells were lodged in the small capillaries of the lung. At day 15 post injection, animals were sacrificed and tumor burden in the lung was quantified by lung mass and immunohistochemistry.

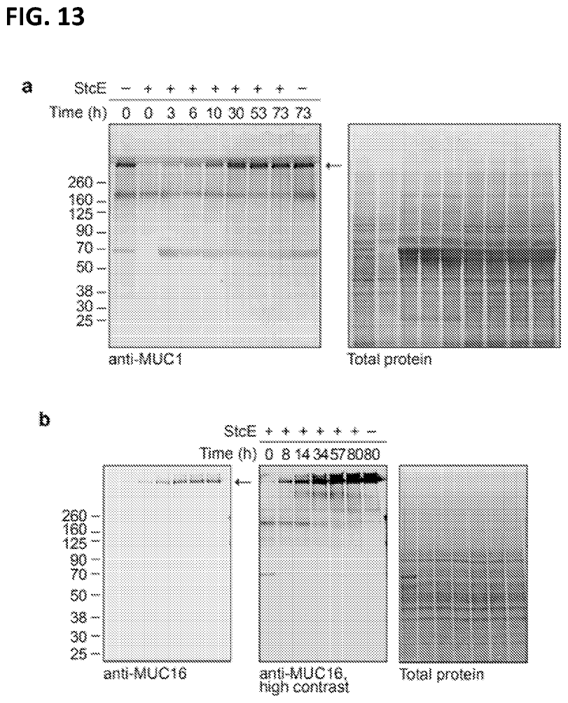

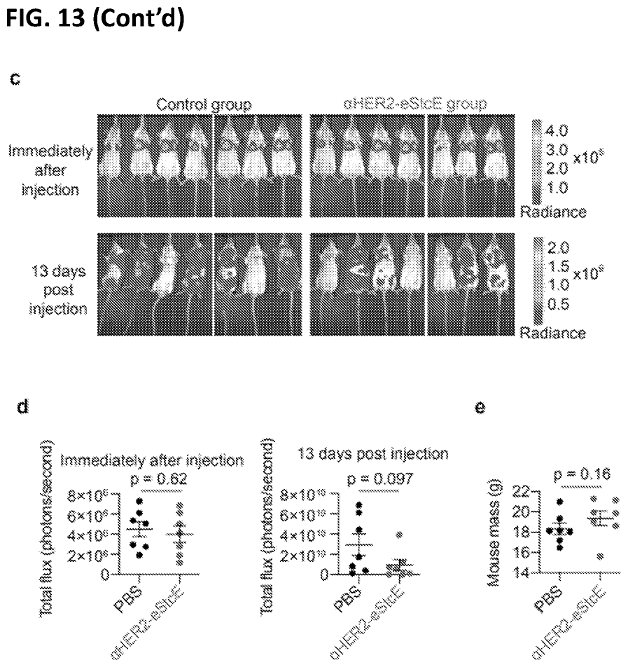

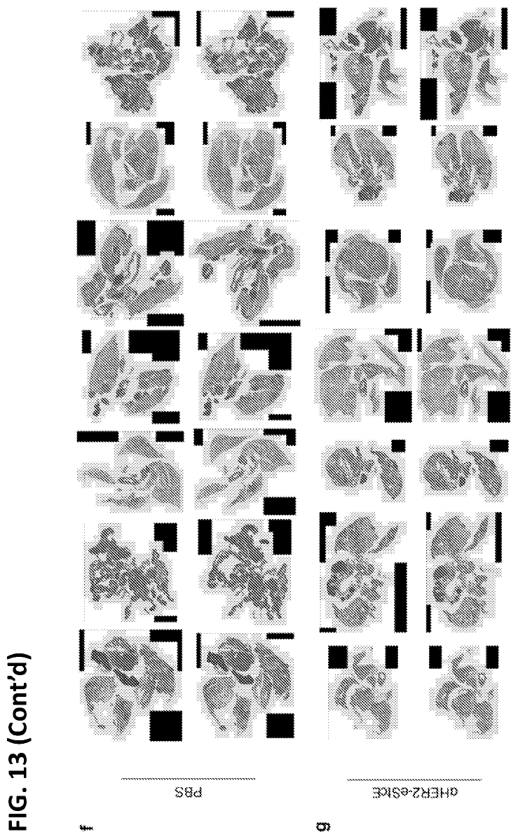

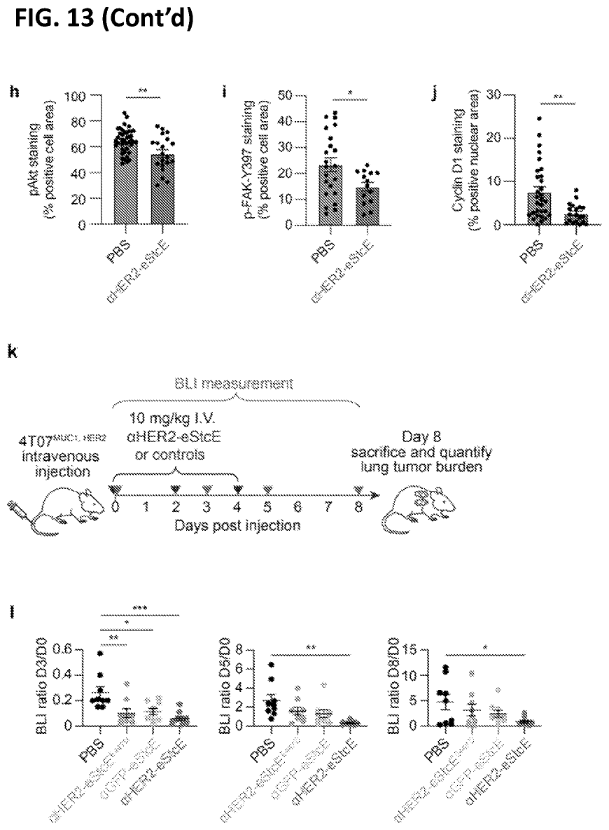

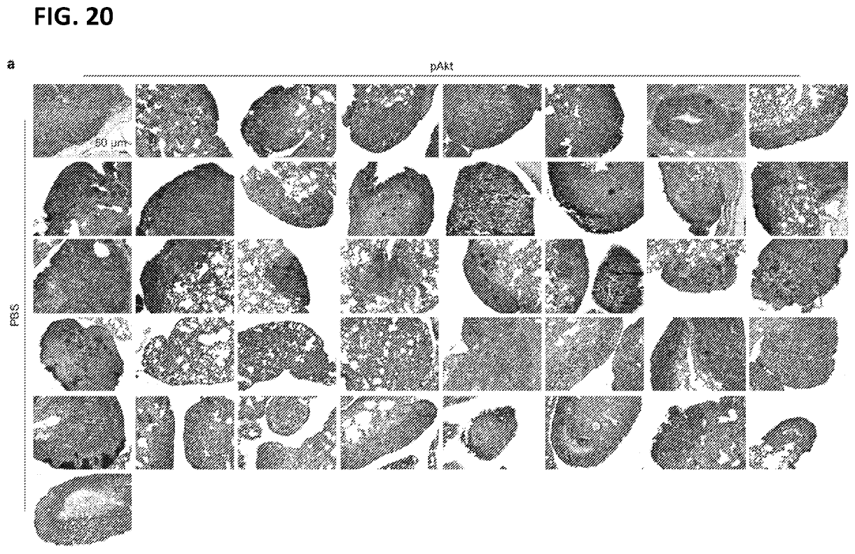

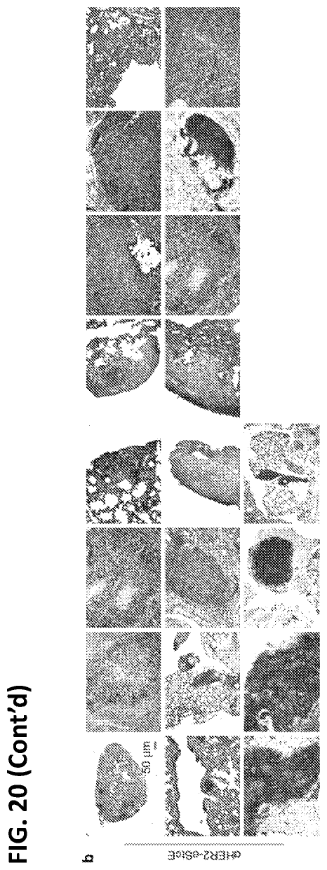

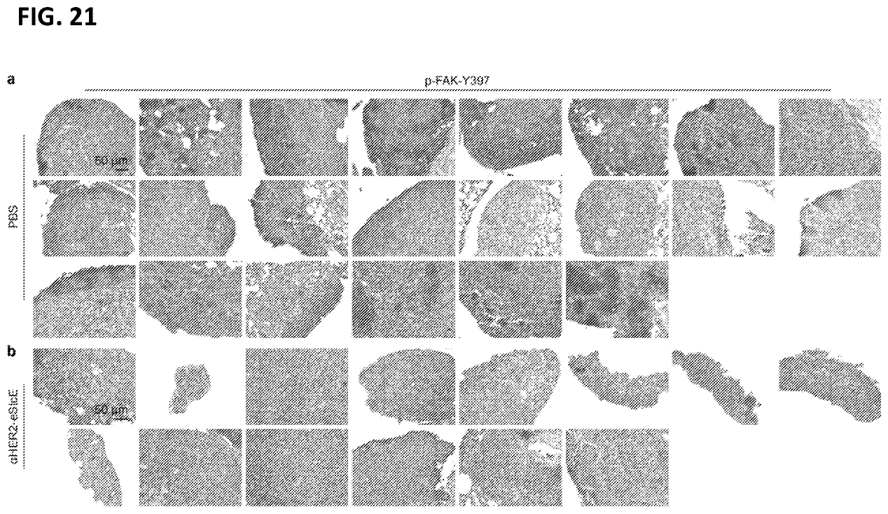

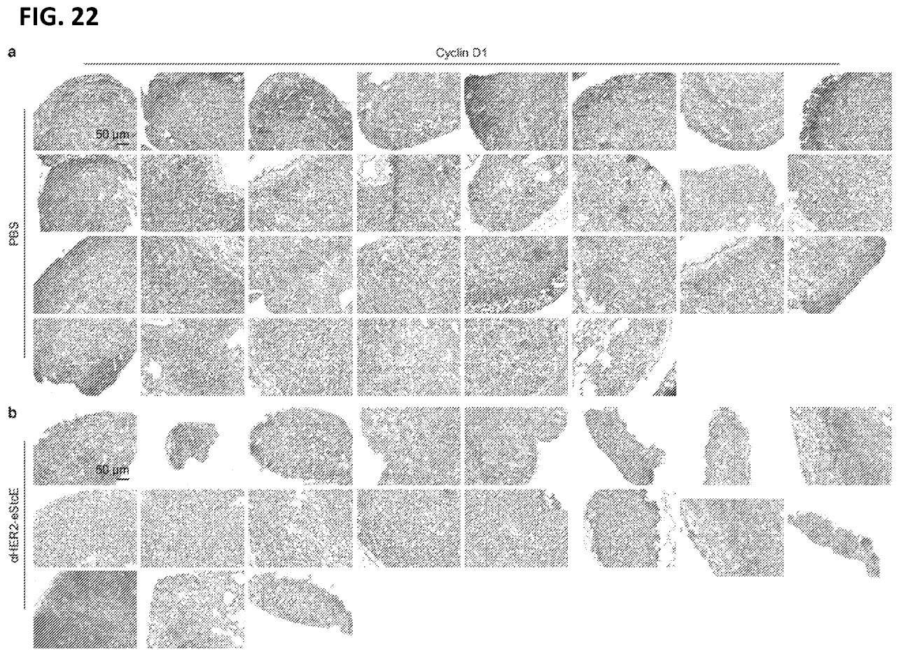

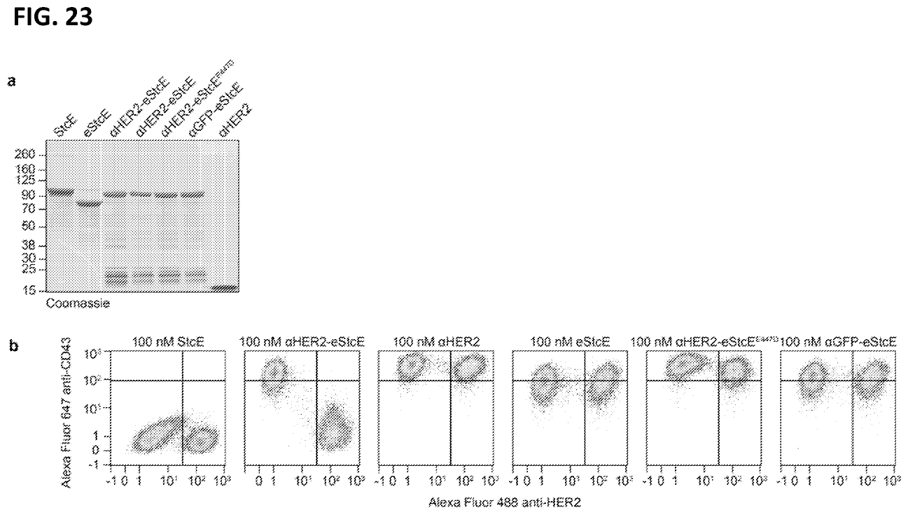

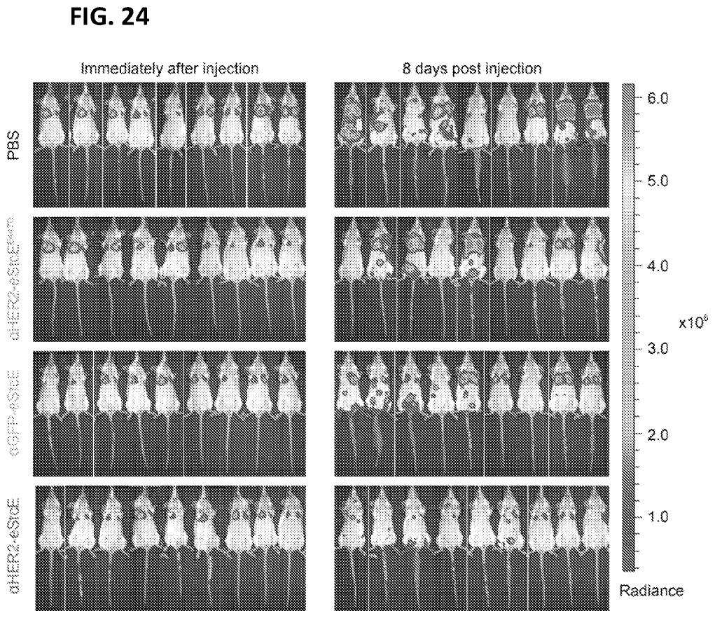

A therapeutic model with 4T07 cells stably expressing MUC1 ectodomain and HER2 was performed, and Balb/c mice were treated every other day with 10 mg/kg αHER2-eStcE or vehicle control (FIG. 4e). The dosing strategy was chosen based on (i) the approximately 24-hour turnover observed in cellulo for enzymatically degraded mucins (FIG. 12a), consistent with reported mucin half-lives³⁴, and (ii) the observed at least 20 hour in vivo circulation time (FIG. 13a-b). Bioluminescent imaging (BLI) directly following injection confirmed 4T07 cells seeded lungs of both control and treatment group animals (FIG. 13c-d). BLI at 13 days post injection revealed reduced tumor burden in αHER2-eStcE treated animals (n=7, p=0.097). Total mouse mass did not differ significantly between groups (FIG. 13e). Necropsy of animals on day 15 post injection showed a decreased wet lung mass (n=7, p=0.0041) and decreased percent metastatic area by IHC (n=7 mice, two lung sections per mouse, p=0.0061) (FIG. 4f-g and FIG. 13f-g). Immunohistochemistry analysis of lungs revealed reduction in cyclin D1 and pFAK-Y397 staining in αHER2-eStcE treated versus control animals (FIG. 13h-j and FIGS. 20-22). These data indicate that treatment with αHER2-eStcE may blunt metastatic growth in the 4T07 model through reversal of mucin ectodomain-driven enhancement of cell cycle progression via the PI3K-Akt axis³³. In a separate experiment in which MUC1 ectodomain expression was maintained through constant administration of doxycycline, efficacy of αHER2-eStcE was tested compared to a non-targeting control, where eStcE was fused to a GFP-targeting nanobody (3OGO)⁵² (termed “αGFP-eStcE”), and an inactive control, with an inactivating mutation to a catalytic residue (termed “αHER2-eStcE^{E447D}”)²⁶. Bioluminescent imaging indicated that while non-targeting and fully inactive controls may initially impair tumor cell survival or enhance tumor cell clearance from the lungs, only targeted αHER2-eStcE treatment produces durable efficacy (FIG. 13k-I and FIGS. 23-24).

The murine cell line EMT6 is a Balb/c syngeneic mammary carcinoma that is used as a model for immune surveillance³⁵. Gray et al. showed that desialylation of orthotopic EMT6 tumors with injected sialidase constructs prolonged the survival of mice through inhibition of the Siglec-sialic acid immune checkpoint axis²⁵. This model involved injection of EMT6 cells into the mammary fat pads of mice followed by I.P. treatment with enzymes or controls. Tumor size was measured with calipers until tumor burden required euthanasia (typically 20-30 days post injection).

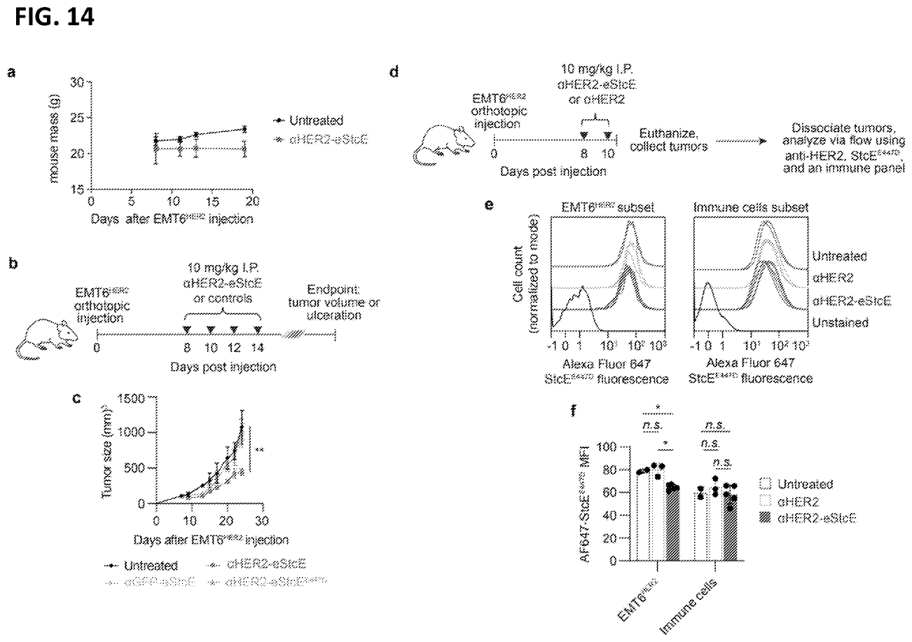

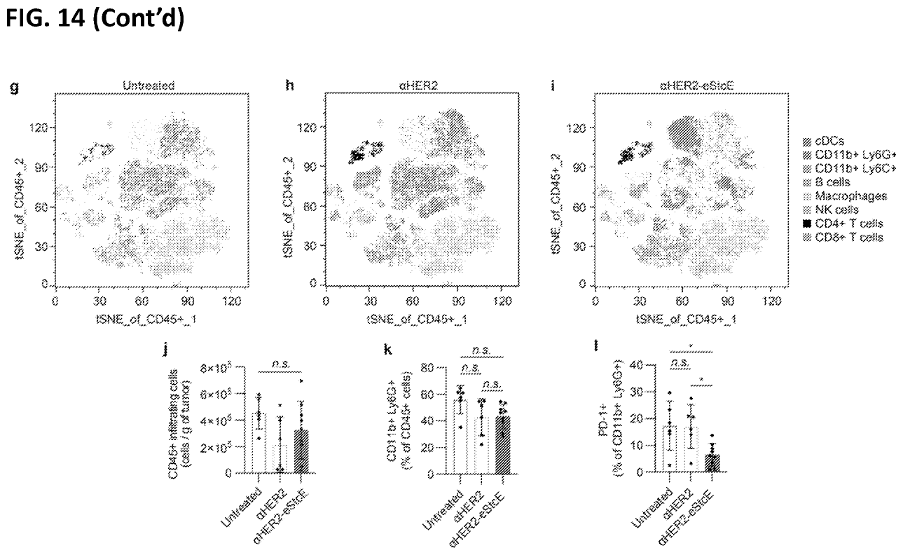

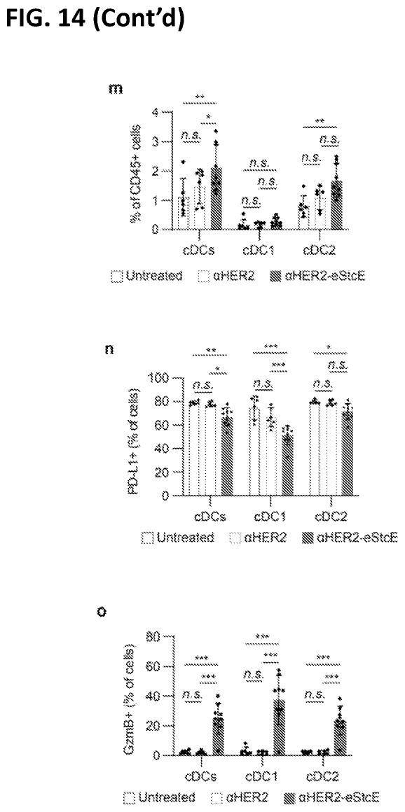

To assess whether mucinase-driven depletion of Siglec ligands from tumor cell surfaces would have a similar beneficial effect, a therapeutic model with EMT6 cells stably expressing HER2 was performed and animals were treated with four doses of 10 mg/kg αHER2-eStcE, an equimolar dose of αHER2, or vehicle control (FIG. 4h). Treatment with αHER2-eStcE resulted in reduced tumor size at day 19 (n=6-9, p<0.0001) (FIG. 4i). After 35 days, all mice in the vehicle-treated group had reached a tumor burden requiring euthanasia, while treatment with αHER2-eStcE extended mouse survival to 47 days (p=0.023 versus control and p=0.0006 versus αHER2 alone) (FIG. 4j). Treatment with αHER2 alone did not result in attenuation of tumor growth or prolonged survival, and mice treated with αHER2-eStcE did not exhibit weight loss over the course of the experiment, suggesting that treatment was well tolerated. (FIG. 14a). In a replicate experiment using a separate set of animals, tumor growth was once again attenuated with treatment of αHER2-eStcE but not with treatment αHER2-eStcE^{E447D} or αGFP-eStcE, indicating that enzymatic activity and tumor-targeting were required for the observed efficacy (FIG. 14b-c).

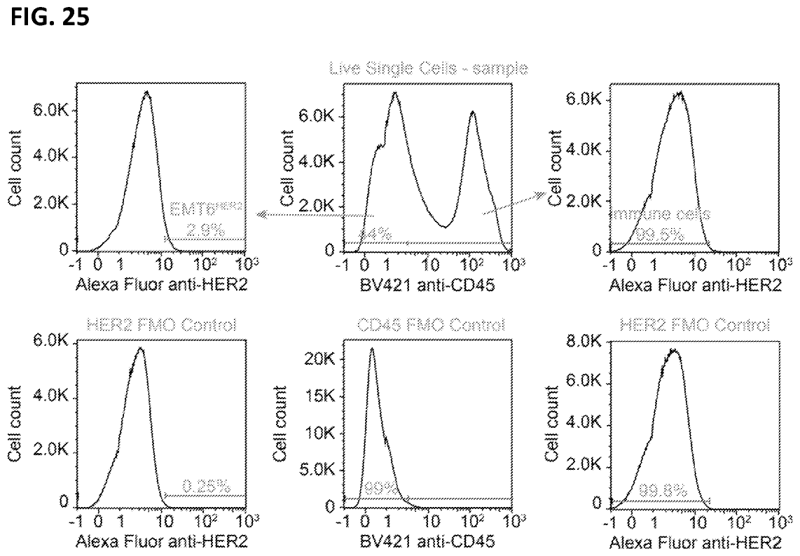

For analysis of mucin degradation and immune infiltration within tumors, a separate set of animals were treated as above with vehicle, αHER2, or αHER2-eStcE, and sacrificed at day 10 post-implantation (FIG. 14d). Flow cytometry analysis of αHER2-eStcE treated animals revealed a modest but significant reduction of cell surface mucins on the EMT6^{HER2} cancer cells (CD45⁻/HER2⁺ cells) without effect on mucin levels on immune cells (CD45⁺/HER2⁻ cells), suggesting αHER2-eStcE promotes selective mucin depletion in vivo (FIG. 14e-f and FIG. 25). EMT6^{HER2} cells and immune cells in animals treated with vehicle or αHER2 control did not exhibit alteration in cell surface mucin levels.

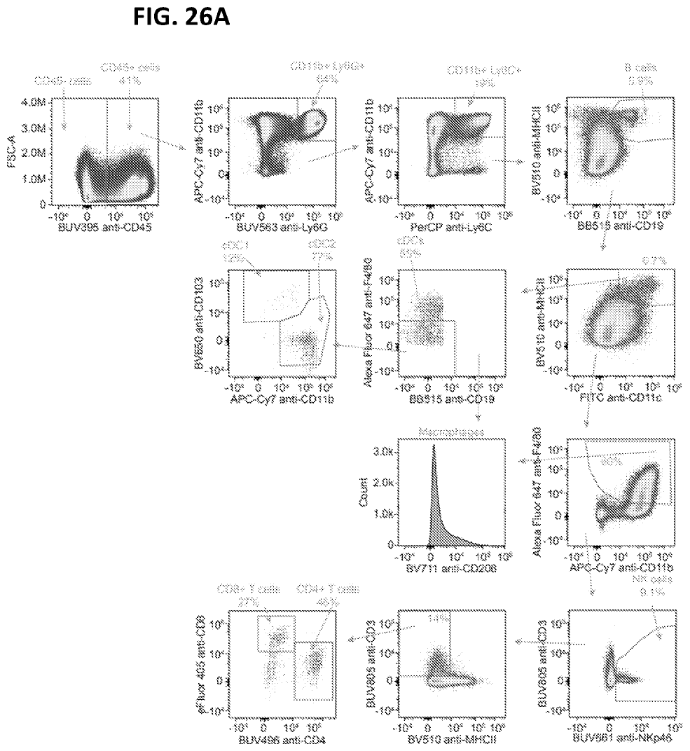

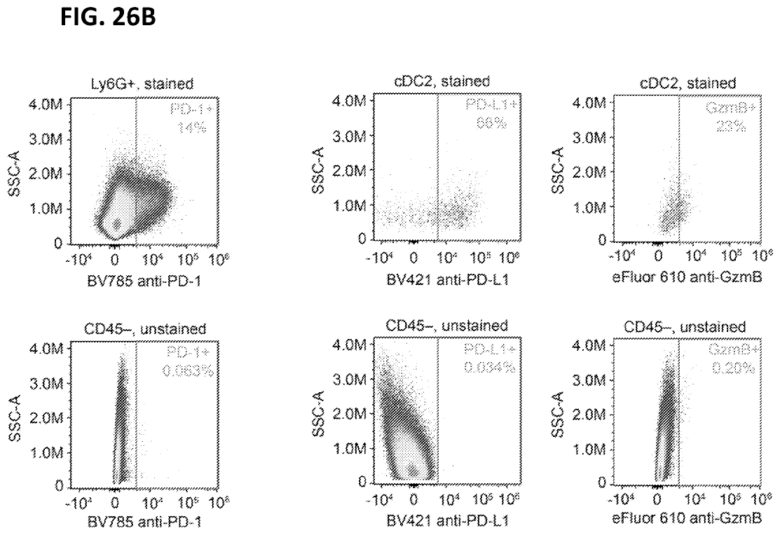

The immune composition within EMT6^{HER2} tumors were profiled and it was found that the dominant immune cell type within these tumors were Ly6G+ cells, which correspond to Ly6G-expressing granulocytes and/or neutrophils that are often found in breast tumor immune infiltrates (FIG. 14g-j and FIG. 26). Tumor-infiltrating Ly6G+ cells from mice treated with αHER2-eStcE showed reduced levels of the inhibitory immune checkpoint PD-1 relative to vehicle and αHER2 treatment groups (FIG. 14k-l). In addition, αHER2-eStcE therapy promoted infiltration of conventional dendritic cells (cDCs) into the tumors (FIG. 14m). We found that cDCs in αHER2-eStcE treated tumors exhibited an augmented phenotype, as indicated by reduced levels of the inhibitory ligand PD-L1 (FIG. 14n). Strikingly, cDCs of conjugated-treated animals also exhibited significantly increased levels of granzyme B, a cytotoxic protease that is released by immune cells to trigger apoptosis of target cells (e.g., cancer cells and virally-infected cells) (FIG. 14o). While granzyme B is typically associated with cytotoxic cells, such as CD8+ T cells and NK cells, it can also be produced by other cell types upon activation. These data, which support modulation of the tumor immune microenvironment following αHER2-eStcE treatment, are consistent with the importance of mucin and sialic acid signaling in the tumor microenvironment.

In summary, targeted degradation of cancer-associated cell surface mucins limits tumor growth in vivo in two disparate murine models of breast cancer progression, with indications that both biophysical and immunological signaling are influenced.

### Methods

Statistical analyses. For all immortalized cell line data, biological replicates refer to experiments performed independently on different days. All statistical methods were performed using Prism (GraphPad). When multiple conditions were compared, a Tukey corrected one-way ANOVA was used. In all circumstances in which multiple groups and conditions were compared, a Tukey-corrected two-way ANOVA was used. For EC50 and Kd comparisons, data were log-normalized prior to statistical comparisons. When two groups were compared, a two-tailed unpaired t-test or multiple unpaired t-tests with two-stage Benjamini, Kreiger, and Yekutieli false discovery rate correction was used. For mouse survival data, a Mantel-Cox test was used.

Cell culture. Cells were maintained at 37° C., 5% CO₂. MCF10AMUC1 cells were cultured in phenol red free 1:1 DMEM: F12 supplemented with 5% New Zealand horse serum (Thermo Fisher Scientific), 20 ng/ml epidermal growth factor (Peprotech), 0.5 μg/mL hydrocortisone (Millipore Sigma), 100 ng/ml cholera toxin (Millipore Sigma), 10 μg/mL insulin (Millipore Sigma), and 1% penicillin/streptomycin (P/S). K562, CCRF-CEM, and 4TO7^{MUC1} cells were cultured in RPMI supplemented with 10% heat inactivated fetal bovine serum (FBS) (Thermo Fisher Scientific) and 1% P/S. HeLa, CCRF-HSB-2, EMT6HER2, and HEK-293T cells were grown in DMEM supplemented with 10% heat inactivated FBS, 10 μg/mL human insulin (Thermo Fisher Scientific), and 1% P/S. MCF7 cells were grown in DMEM supplemented with 10% heat inactivated FBS and 1% P/S and 10 g/mL human insulin (Thermo Fisher Scientific). OVCAR-3N cells were cultured in RPMI supplemented with 10% heat inactivated FBS, 0.01 mg/mL bovine insulin (Sigma-Aldrich), and 1% P/S. Cells were counted using Countess II FL Automated Cell Counter (Thermo Fisher Scientific) following manufacturer's recommendations.

TABLE 5
Antibody clones and working concentrations.

| Antibody | Vendor, clone | Dilution/concentration |
|---|---|---|
| AF488/647 anti-human CD340 | BioLegend, 24D2 | 1:200 |
| (erbB2/HER-2) antibody | | |
| AF647 CD43/sialophorin antibody | Novus Biologicals, MEM-59 | 1:1000 (flow cytometry |
| | | on BD Accuri C6 plus) |
| | | 2.5 μg/mL (flow |
| | | cytometry on |
| | | MACSQuant10) |
| Anti-MUC1/episialin antibody | EMD Millipore, 214D4 | 1:200 (flow cytometry), |
| | | 1:1000 (Western blot) |
| AF647 Affinipure Goat Anti-Mouse IgG | Jackson ImmunoResearch | 1:375 |
| FITC anti-His antibody | Miltenyi Biotec, GG11-8F3.5.1 | 1:50 |
| MUC1 mouse mAb | Cell Signaling Technology, VU4H5 | 1:200 |
| MUC16 mouse mAb | Abcam, X75 | 1:1000 |
| InVivoMAb anti-mouse/human/rat CD47 | BioXCell, MIAP410 | 20 μg/mL |
| BV421 anti-mouse CD45 antibody | BioLegend, 30-F11 | 1:80 |
| Purified Rat Anti-Mouse CD16/CD32 | Clone 2.4G2 | 1:50 |
| (Mouse BD Fc Block) | | |
| Anti-phospho-FAK tyrosine 397 | Invitrogen, Cat. #: 700255 | 1:100 |
| Anti-Cyclin D1 | Cell Signaling Technology, Cat. #: 2922 | 1:200 |
| Anti-Phospho-(Ser/Thr) Akt Substrate Antibody | Cell Signaling Technology, Cat. #: 9611 | 1:400 |

TABLE 6
Flow panel antibodies related to FIG. 14g-o and FIG. 26.

| Antibody | Color | Vendor, Cat. No. | Cat. Number | Dilution |
|---|---|---|---|---|
| CD45 | BUV395 | BD Biosciences | 564279 | 1:100 |
| Live-dead | Zombie UV | BioLegend | 423107 | 1:100 |
| CD4 | BUV496 | BD Biosciences | 93937 | 1:100 |
| Ly-6G | BUV563 | BD Biosciences | 612921 | 1:200 |
| NKp46 | BUV661 | BD Biosciences | 741678 | 1:70 |
| CD3 | BUV805 | BD Biosciences | 749276 | 1:70 |
| PD-L1 | BV421 | BioLegend | 124315 | 1:150 |
| LFA-1 | SB436 | eBioscience | 62011180 | 1:100 |
| CD8 | eFluor 450 | eBioscience | 480081 | 1:100 |
| MHCII | BV510 | BioLegend | 107635 | 1:300 |
| CD80 | BV605 | BioLegend | 104729 | 1:70 |
| CD103 | BV650 | BD Biosciences | 748256 | 1:70 |
| CD206 | BV711 | BioLegend | 141727 | 1:100 |
| PD-1 | BV785 | BioLegend | 135225 | 1:100 |
| CD19 | BB515 | BD Biosciences | 564509 | 1:100 |
| CD11c | FITC | BioLegend | 117306 | 1:100 |
| Ly-6C | PerCP | BioLegend | 128028 | 1:200 |
| Tim-3 | BB700 | BD Biosciences | 747619 | 1:100 |
| Siglec E | PE | BioLegend | 677104 | 1:50 |
| CD25 | PE-Cy5.5 | eBioscience | 35025182 | 1:100 |
| F4/80 | AF647 | BioLegend | 123122 | 1:100 |
| CD11b | APC-Cy7 | BioLegend | 101226 | 1:100 |
| Ki67 | AF532 | eBioscience | 58569882 | 1:200 |
| TCF-7 | AF700 | R&D Systems | FAB82224N | 1:100 |
| GzmB | PE-eFluor610 | eBioscience | 61889882 | 1:100 |
| FoxP3 | APC | eBioscience | 17577382 | 1:100 |

MCF10A^{MUC1} suspension survival assay. MCF10A cells expressing a cytoplasmic truncation 5 of MUC1 (MUC1ΔCT, also referred to as MUC1 ectodomain) were used to limit any possible cytoplasmic signaling¹. MUC1ΔCT was induced with 200 ng/ml doxycycline for 24 h. Uninduced and induced cells were seeded at 3×10⁵ cells/well in a 24-well ultra-low attachment plate (Corning) in 0.75 mL of complete media. 200 ng/mL doxycycline and 10 nM StcE were added as appropriate. The plate was incubated at 37° C., 5% CO₂, 125 rpm. At t=0, 24, 48, and 72 h, cells were spun down at 350×g for 5 min, resuspended in 200 μL of phosphate-buffered saline (PBS) with 0.1% benzonase (Sigma-Aldrich), and incubated at room temperature for 15 min. Cells were then resuspended in 200 μL of enzyme-free cell dissociation buffer (Thermo Fisher Scientific) and stained with 100 nM Calcein AM (Thermo Fisher Scientific) and 5 nM Sytox Red Dead Cell Stain (Thermo Fisher Scientific) for 20 min at 4° C., prior to analysis using a BD Accuri C6 plus. Videos of MCF10A^{MUC1} cells freshly seeded on standard tissue culture plates (Corning) and treated with and without 1 nM StcE were generated with images taken at 30 min intervals for 18 h using an Incucyte. The Incucyte was set to 37° C. and 5% CO₂, and cells were incubated in complete media with 200 ng/ml doxycycline.

For mixed cell assays, MUC1ΔCT was induced with 200 ng/ml doxycycline for 24 h. A 1:1 mixture of 2.5×10⁵ MCF10A^{MUC1} and MCF10AMUC1, HER2 cells were seeded per well in a 24-well ultra-low attachment plate in 0.80 mL of complete media. 200 ng/ml doxycycline, 1 nM StcE, and 1 nM αHER2-eStcE were added as appropriate. The plate was incubated at 37° C., 5% CO₂, 125 rpm. At t=0, 24, 48, and 72 h, cells were spun down at 350× g for 5 min, resuspended in 100 μL of PBS with 0.1% benzonase, and incubated at room temperature for 15 min. Cells were then resuspended in 200 μL of enzyme-free cell dissociation buffer and stained with Alexa Fluor 647 anti-human CD340 (erbB2/HER-2) antibody (24D2 clone) (BioLegend) and 500 nM Sytox Green Nucleic Acid Stain (Thermo Fisher Scientific) for 20 min at 4° C., according to manufacturer recommendations, prior to analysis using a BD Accuri C6 plus. All flow cytometry data were analyzed using FlowJo v. 10.0 (TreeStar).

CD43 and Siglec-7-Fc flow cytometry. 1×10⁶ K562, CCRF-CEM, or CCRF-HSB-2 cells growing in log phase were harvested, resuspended in 1 mL of serum-free RPMI, and treated with either vehicle or 50 nM StcE for 1 h. Cells were subsequently spun down at 600× g and washed twice in PBS. Cells were then resuspended in FACS buffer (0.5% BSA in PBS) at 1×10⁶ cells/mL and aliquoted into a V-bottom 96-well plate (Corning) at 1×10⁵ cells/well. For staining, a precomplex solution of 1 μg/mL Siglec-7-Fc (R&D Systems) and 1 μg/mL Alexa Fluor 488-antihFc was made up in FACS buffer and incubated on ice for 1 h. Alexa Fluor 647 CD43/sialophorin antibody (MEM-59 clone) (Novus Biologicals) was subsequently added to the precomplex solution prior to staining. Cells were stained in 100 μL of staining solution for 30 min, washed twice with FACS buffer, and analyzed by flow cytometry using a BD Accuri C6 plus.

Human donor-derived macrophage isolation. Peripheral blood mononuclear cells (PBMCs) were isolated from LRS chambers (Stanford Blood Center) using a Ficoll-Paque density gradient (Cytiva). Isolated PBMCs were extracted from the PBS/Ficoll interface and washed three times with PBS. PBMCs were resuspended in RPMI containing 10% heat inactivated FBS and plated at 1×10⁷cells/well into a 24-well #1.5 glass plate (Cellvis) that was pre-coated with poly-L-lysine solution (Millipore Sigma). PBMCs were incubated for 1 h at 37° C. to allow monocytes to adhere to the glass. Cells were then rinsed three times with PBS to remove contaminating lymphocytes. Media was replaced with IMDM containing 10% human AB serum (Gemini). Monocytes were differentiated for 7-9 days.

NK cell isolation. PBMC aliquots were quickly thawed and diluted in 10 mL of RPMI containing DNAase to break up cell aggregates. Cells were incubated at 5% CO₂, 37° C. for 30 min and subsequently counted in duplicate. Cells were then spun down at 600× g and resuspended in RPMI to a final cell concentration of 50×10⁶ cells/mL. Isolation of NK cells was performed according to manufacturer's instructions using an NK cell magnetic isolation kit (Stem Cell Technologies). NK cells were cultured for at least 24 h before conducting experiments. For killing experiments, NK cells were cultured for 24 h in complete media containing 0.2-0.5 μg/mL IL-2 (BioLegend).

NK cell killing assays. Target cells were harvested by centrifugation and resuspended in serum-free RPMI containing 5 μM Cell Tracker Far Red (Thermo Fisher Scientific) at 5×10⁵ cells/mL. Cells were then incubated for 30 min at 37° C.; where indicated, cells were treated with 10⁻²⁰ nM StcE. Following staining and StcE treatment, cells were spun down, washed twice with PBS containing 1 mM EDTA, and resuspended in complete media. Cells were diluted to 1×10⁵ cells/mL in complete media containing 100 nM Sytox Green, and 100 μL of cell suspension was aliquoted into a flat bottom 96-well plate. Separately, NK cells were diluted to various cell concentrations to generate the indicated effector: target ratios in complete media containing 100 nM Sytox Green. Where indicated, these cell suspensions were treated with 20 nM StcE for 30 min, washed twice with PBS containing 1 mM ETDA, and resuspended in complete media containing 100 nM Sytox Green. 100 μL of these cell suspensions was then mixed with the target cell suspensions to generate a total volume of 200 μL. Cells were incubated at 37° C. for 4 h and analyzed by flow cytometry.

For mixed cell assays, K562^{HER2} cells were harvested by centrifugation and incubated for 30 min at 37° C. in serum-free RPMI containing 0.33 μM CellTrace Far Red (Thermo Fisher Scientific) at 5×10⁵ cells/mL. K562, isolated NK, and K562^{HER2} cells were harvested by centrifugation and resuspended in complete media. 1×10⁴ K562 cells, 1×10⁴ K562^{HER2} cells, and 2×10⁴ NK cells in 200 μL of complete media containing 50 nM Sytox Green were added to a flat bottom 96-well plate. PBS, αHER2-eStcE, or StcE in PBS were added to a final volume of 222.2 μL and incubated for 4 h at 37° C. prior to analysis by flow cytometry using a BD accuri C6 plus.

Bioactive compound library screen. A library of 261 bioactive compounds (Selleck Chemicals) was stored at −80° C. The library was re-formatted from 96-well to 384-well format using a Versette automated liquid handler configured with a 96-channel pipetting head and diluted to 2 mM in DMSO. The day before the screen, 5×10³ OVCAR-3^{N} cells/well were seeded into two 384-well plates in 45 μL of medium. The next day, the medium was removed and replaced with medium containing 20 nM Sytox Green and compounds from a freshly thawed library master stock plate (1 compound/well) were added to a final concentration of 500 nM. One plate was co-treated with vehicle (PBS) and the other with 50 nM StcE. Plates were imaged immediately and every 2 h thereafter for a total of 72 h using the Essen IncuCyte Zoom. Counts of Sytox Green and mKate² objects per mm² were obtained and the lethal fraction calculated as previously described². The area-under-the-curve (AUC) of lethal fraction scores across the full 72 h were calculated using the trapezoid rule in Excel (Microsoft Corp.). The Bliss Independence Model was used to compute expectant cell death of compound+ StcE treatment using normalized AUC (nAUC) values, and deviation from this expectation was used to infer modulation of cell death, as described previously³.

Measuring cell death using STACK. Follow-up cell death experiments of OVCAR-3^{N} cells were performed using scalable time-lapse analysis of cell death kinetics (STACK) 2. Cell lines stably expressing nuclear-localized mKate² (Nuc::mKate²) were incubated in medium containing 20 nM Sytox Green. Counts of live (mKate²⁺) and dead (SG⁺) objects were obtained from images collected every 2 or 4 h. The following image extraction parameter values were used to count OVCAR-3^{N} mKate²⁺ objects: Parameter adaptive, threshold adjustment 1; Edge split on; Edge sensitivity 50; Filter area min 20 μm², maximum 8100 μm²; Eccentricity max 1.0; and SG⁺ objects: Parameter adaptive, threshold adjustment 10; Edge split on; Edge sensitivity-5; Filter area min 20 μm², maximum 750 μm²; Eccentricity max 0.9. Counts were exported to Excel and lethal fraction (LF) scores were computed from mKate²⁺ and SG⁺ counts as described². To compute LF, double mKate²/Sytox Green positive counts were subtracted from live cell counts.

ColabFold modeling. Protein sequences for StcE and αHER2-eStcE were used as inputs for ColabFold (https://colab.research.google.com/github/sokrypton/ColabFold/blob/main/AlphaFold².ipynb #scrollTo=kOblAo-xetgx) to obtain model structures⁴⁷. The predicted structure for StcE aligned well (for previously reported StcE structure²⁶ RMSD=0.2) with the X-ray structure determined by Yu et al.²⁶. Molecular graphics were generated using PyMOL.

Molecular modeling. The AlphaFold-predicted structure of StcE⁵³ was overlaid with a glycopeptide-StcE model complex¹² previously generated via docking experiments with the crystal structure originally determined by Yu et al.²⁶ As such, the unaltered glycopeptide ligand Ac-P(GalNAc) TLTH-NMe and zinc ion of the docked complex underwent brief minimization with the AlphaFold structure using the Amber¹⁰:EHT forcefield⁵⁴ in Molecular Operating Environment to yield the final complex used to inform mutagenesis studies.

Cloning. StcE mutants were cloned from pET28b-StcE_Δ35-NHis, generously provided by Natalie Strynadka (University of British Columbia), using a Q5 Site-Directed Mutagenesis Kit (New England Biolabs), In-Fusion HD Cloning Plus (Takara Bio), or from ordering related designed plasmids from Twist Bioscience. All plasmids were sequence confirmed (Elim Biopharm) before proceeding. The amino acid sequence for the 5F7 nanobody was provided by Melissa Gray⁴, and 3OGO nanobody (αGFP) (previously published⁵²) were reverse translated, and optimized for expression in Escherichia coli K12 with the IDT Codon Optimization Tool before cloning as above. A plasmid containing the sequence for TP1107 (αIgG1) was ordered from Addgene (plasmid #104158) and cloned into the plasmid as above.

Protein purification. BL21 (DE3) Escherichia coli were transformed with sequence confirmed plasmids and grown in sterile terrific broth with 30 μg/mL kanamycin at 37° C., 250 rpm until an optical density of 0.4-0.8 was reached. Protein expression was induced with 0.3 mM IPTG and the culture was incubated overnight at 20° C., 250 rpm. Cells were spun down at 6000× g for 10 min and lysed in 20 mM HEPES, pH 7.5, 500 mM NaCl with a probe tip sonicator. Lysates were clarified by spinning at 11,000×g for 10 min and filtered through a low protein binding 0.22 μm polyethersulfone membrane vacuum filter bottle (Corning). Lysates were applied to 3-4 mL of Ni-NTA agarose (Qiagen) per liter of bacterial culture, washed with 200 ml of 20 mM HEPES, pH 7.5, 500 mM NaCl, 20 mM imidazole, and eluted with 20 mL of 20 mM HEPES, pH 7.5, 500 mM NaCl, 250 mM imidazole per liter of culture. Purified proteins were buffer exchanged into cold PBS either with Zeba Spin Desalting Columns, 7K MWCO, 0.5 mL capacity (Fisher Scientific) or through dialysis with Pierce Slide-A-Lyzer G2 Dialysis Cassettes, 20K MWCO (Fisher Scientific). Protein concentration was determined via nanodrop and protein purity was determined by SDS-PAGE. Purified protein aliquots were stored at −80° C. and thawed and stored at 4° C. before experiments.

Endotoxin-free protein purification. ClearColi BL21 (DE3) Electrocompetent Cells (Lucigen) were transformed with plasmids and grown in sterile LB-Miller Culture Media with 30 μg/mL kanamycin at 37° C., 250 rpm until an optical density of 0.4-0.8 was reached. Protein expression was induced with 0.4 mM IPTG and the proteins were prepped as above. Proteins were run through Pierce high-capacity endotoxin removal columns (ThermoFisher Scientific) at least four times following manufacturer's instructions. Endotoxin levels were tested using HEK-Blue™ LPS Detection Kit 2 (Invivogen) according to manufacturer recommendations. All endotoxin levels were confirmed to be below K/M for the maximum dose used in vivo, where K is 5 EU/kg and M is the dose of the protein/formulation of interest within a single hour period⁵.

In vitro mucin cleavage activity assays. Recombinant C1-INH (Molecular Innovations) was labeled with IRDye 800CW NHS Ester (LI-COR Biosciences) (dye: protein ratio of 0.54) following manufacturer's instructions. Extra dye was removed with Zeba Spin Desalting Columns, 7K MWCO, 0.5 mL capacity (Fisher Scientific). For each reaction, 50 nM of mucinase and 500 nM of recombinant mucin in PBS were combined and incubated at 37° C. for 1 h. 4X NuPAGE LDS Sample Buffer (Fisher Scientific) and dithiothreitol (DTT) (Thermo Fisher Scientific) were added to a final concentration of 1-2X and 1-250 mM, respectively, and boiled at 95° C. for 5 min. Samples were run on a 4 to 12% 18-well Criterion XT Bis-Tris protein gel (Bio-Rad) in XT MOPS (Bio-Rad) at 180 V for 1 h. Gels were imaged using an Odyssey CLx Near-Infrared Fluorescence Imaging System (LI-COR Biosciences). To quantify mucinase activity, product band and C1-INH parent band signal intensities were determined on Image Studio software, and digestion percentage was calculated by dividing the signal from the product bands by the signal from the parent+ product bands. For rhMUC16 digestion experiments, recombinant MUC16 (R&D Systems) was left unlabeled but otherwise reacted as above. Protein was visualized with AcquaStain Protein Gel Stain (Bulldog-Bio), washed at least three times with ddH₂O, and imaged using an Odyssey CLx Near-Infrared Fluorescence Imaging System. For comparison of in vitro substrate digestion efficiency of different mutants, 89 μg/mL of unlabeled substrates and 162 nM of mucinase (StcE, StcE^{W366A}, ddStcE, WA, or αHER2-eStcE) in PBS were combined and incubated overnight at 37° C. SDS-PAGE gels were run and analyzed as above. Bovine serum albumin (BSA) was purchased from Sigma-Aldrich (A7906-1 KG). Fetuin was purchased from Promega (V4961). Recombinantly expressed MUC16, podocalyxin, CD43, and PSGL-1 were purchased from R&D Systems (5609-MU, 1658-PD, 9680-CD, 3345-PS, respectively.

In cellulo MUC1 cleavage activity assay. Hela cells resuspended at 4.5-5.0×10⁵ cells in 150 μL of complete media were allocated per well to a 96-well ultra-low attachment round bottom plate (Corning). 50 μL of the different StcE mutants in PBS were added to the wells, and the plate was incubated at 37° C. for 1 h. Cells were washed twice with cold FACS buffer containing 2 mM EDTA, once with cold FACS buffer, and stained with anti-MUC1/episialin antibody (clone 214D4) (EMD Millipore) in FACS buffer supplemented with 0.1% benzonase for 30 min on ice. Cells were washed three times with cold FACS buffer containing 2 mM EDTA and stained with Alexa Fluor 647 Affinipure Goat Anti-Mouse IgG (Jackson ImmunoResearch). Cells were washed twice with FACS containing 2 mM EDTA and stained with 30 nM Sytox Green for 10 min at 4° C. prior to analysis using a BD Accuri C6 plus. For data analysis, the Alexa Fluor 647 mean fluorescence intensity of unstained and PBS-treated samples were used to define 0% and 100% cell surface MUC1, respectively, and each sample was normalized to percent MUC1 within each replicate. Using GraphPad Prism 9, each replicate was fitted to inhibitor concentration vs normalized response and log₁₀ (IC₅₀) was reported.

Mucinase binding assays to cell surface mucins. 4-5×10⁵ Hela cells were added to each well of a V-bottom 96-well plate and washed three times with cold FACS buffer containing 2 mM EDTA. Cells were treated with mucinases in cold FACS buffer containing 2 mM EDTA and 0.1% benzonase for 30 min on ice, washed three times with FACS with 2 mM EDTA, and stained with FITC anti-His antibody (clone GG11-8F3.5.1) (Miltenyi Biotec). Cells were washed three times with FACS buffer with 2 mM EDTA and stained with 5 nM Sytox Red in FACS buffer with 2 mM EDTA for 20 min prior to analysis using a MACSQuant Analyzer 10 Flow Cytometer (Milyteni Biotec). Within each sample, binding was normalized to the greatest mean fluorescence intensity (MFI) per mucinase, with 0 being defined as the MFI for the PBS-treated sample. To mitigate the hook effect, only concentrations with greater than 85% normalized binding past the maximum signal were included. In GraphPad Prism 9, each replicate was fitted to agonist concentration vs response with the lower limit restricted to 0 and log₁₀ (EC₅₀) was reported. For conjugate and nanobody binding assays, MCF10A^{±MUC1±HER2} cells were processed, washed, and stained as described above. Cells were analyzed on a BD Accuri C6 plus. For replicates in which Prism could not correctly fit the data to report an EC₅₀ value, the replicate was not included in the bar graph of EC₅₀ values; this occurred with one replicate of αHER2-eStcE binding to MCF10A.

ddStcE^{W366A} mass spectrometry sample preparation. Recombinantly expressed MUC16, podocalyxin, CD43, and PSGL-1 were purchased from R&D Systems (5609-MU, 1658-PD, 9680-CD, 3345-PS, respectively). All recombinant mucin-domain glycoproteins were reconstituted in ultrapure water (Pierce) to a concentration of 1 mg/mL. A fraction (1 μg; 1 μL) of each recombinant glycoprotein was digested with ddStcE^{W366A} at a 1:1 enzyme-to-substrate (E: S) ratio. For sialidase-treated samples, 1 μL of sialoEXO (Genovis) was added to 39 μL of ultrapure water, and 1 μL of this dilution was added to the reaction vial, per manufacturer instructions. The reaction was brought to a total volume of 12 μL in 50 mM ammonium bicarbonate and allowed to react overnight at 37° C. Control reactions were incubated at 37° C. overnight in a solution containing buffer only. The following day, the volume was increased to 19 μL with 50 mM ammonium bicarbonate. PNGaseF (1 μL; Promega) was added to 99 μL of 50 mM ammonium bicarbonate, and 1 μL of this reaction was added to each mucinase reaction vial. De-N-glycosylation reactions were incubated for 8-12 h at 37° C. Reduction and alkylation were performed according to ProteaseMax (Promega) protocols. Briefly, the solution was diluted to 93.5 μL with 50 mM ammonium bicarbonate. Then, 1 μL of 0.5 M dithiothreitol (DTT) was added and the samples were incubated at 56° C. for 20 min, followed by the addition of 2.7 μL of 0.55 M iodoacetamide at room temperature for 15 min in the dark. Digestion was completed by adding sequencing-grade trypsin (Promega) at a 1:20 E: S ratio overnight at 37° C. and quenched by adding 0.3 μL of glacial acetic acid. C18 clean-up was performed using 1 mL strataX columns (Phenomenex). Each column was wet with 1 mL of acetonitrile once, followed by one 1 mL rinse of buffer A (0.1% formic acid in water). The samples were diluted to 1 mL in buffer A and loaded through the column, then rinsed with buffer A. Finally, the samples were eluted with three rinses of 100 μL of buffer B (0.5% formic acid, 80% acetonitrile) and dried by speedvac. The samples were reconstituted in 10 μL of buffer A for MS analysis.

Mass spectrometry for cleavage motif. Samples were analyzed by online nanoflow liquid chromatography-tandem mass spectrometry using an Orbitrap Fusion Tribrid mass spectrometer (Thermo Fisher Scientific) coupled to a Dionex Ultimate 3000 HPLC (Thermo Fisher Scientific). Each sample was analyzed twice; once with an HCD triggered electron transfer dissocitiaton (ETD) method (for input to Byonic), and the second with an HCD triggered EThcD method (for input into OPair). A portion of the sample (4 μL of 10 μL; 40%) was loaded via autosampler isocratically onto a C18 nano precolumn using 0.1% formic acid in water (“solvent A”). For preconcentration and desalting, the column was washed with 2% acetonitrile and 0.1% formic acid in water (“loading pump solvent”). Subsequently, the C18 nano precolumn was switched in line with the C18 nano separation column (75-μm×250-mm EASYSpray containing 2 μm C18 beads) for gradient elution. The column was held at 40° C. using a column heater in the EASY-Spray ionization source (Thermo Fisher Scientific). The samples were eluted at a constant flow rate of 0.3 μL/min using a 90 min gradient. The gradient profile was as follows (min: % solvent B, 2% formic acid in acetonitrile): 0:3, 3:3, 93:35, 103:42, 104:95, 109:95, 110:3, 140:3. The instrument method used an MS1 resolution of 60,000 full width at half maximum (FWHM) at 400 m/z, an automatic gain control (AGC) target of 3e5, and a mass range from 300 to 1,500 m/z. Dynamic exclusion was enabled with a repeat count of 3, repeat duration of 10 s, and exclusion duration of 10 s. Only charge states 2 to 6 were selected for fragmentation. MS2s were generated at top speed for 3 s. Higher-energy collisional dissociation (HCD) was performed on all selected precursor masses with the following parameters: isolation window of 2 m/z, 30% collision energy, orbitrap detection (resolution of 30,000), and an AGC target of 1e⁴ ions. For HCD-pd-ET (hc) D runs, ET (hc) D was performed if 1) the precursor mass was between 300 and 1,000 m/z and 2) 3 of 9 HexNAc or NeuAc fingerprint ions (126.055, 138.055, 144.07, 168.065, 186.076, 204.086, 274.092, and 292.103) were present at +0.1 m/z and greater than 5% relative intensity. ETD parameters were as follows: calibrated charge-dependent ETD times, 2e⁵ reagent target, and precursor AGC target 1e⁴. EThcD parameters were the same but included 30 nce supplemental activation and Orbitrap analysis at a resolution of 30,000 FWHM.

Mass spectrometry data analysis for cleavage motif. HCD-pd-ETD raw files were searched using Byonic by ProteinMetrics against directed databases containing the recombinant protein of interest. Search parameters included semi-specific cleavage specificity at the C-terminal site of R and K, meaning non-tryptic cleavage was permitted at either the N- or C-terminus of a detected peptide but not both. Mass tolerance was set at 10 ppm for MS1s, 0.1 m/z for HCD MS2s, 0.35 m/z for ETD MS2s. Methionine oxidation (common 2), asparagine deamidation (common 2), and N-term acetylation (rare 1) were set as variable modifications with a total common max of 3, rare max of 1. O-glycans were also set as variable modifications (common 2), using the “O-glycan 6 most common” database. Cysteine carbaminomethylation was set as a fixed modification. Peptide hits were filtered using a 1% FDR. All peptides were manually validated and/or sequenced using Xcalibur software (Thermo Fisher Scientific). HCD was used to confirm that the peptides were glycosylated and ETD spectra were used for site-localization of glycosylation sites.

To further confirm ddStcE^{W366A} cleavage sites, HCD-pd-EThcD raw files were searched using O-Pair in Metamorpheus against directed databases containing the recombinant protein of interest. Search parameters included an “O-glycopeptide search” using the “Oglycan.gdb” database. The top 50 candidates were kept, using HCD-pd-EThcD fragmentation, with a maximum of 4 glycans allowed. Semi-trypsin cleavage specificity was selected with a maximum of 2 missed cleavages, and a peptide length of 5-60. Mass tolerance was set at 10 ppm for MS1s, and 20 ppm for all MS2s. Cysteine carbaminomethylation was set as a fixed modification, and methionine oxidation and asparagine deamidation were set as variable modifications. O-Pair results were filtered for results with a Q value of less than 0.01.

TAILS mass spectrometry sample preparation. TAILS methods were adapted from previous TAILS publications^{6,7} and protocols available at the Overall group website, clip.ubc.ca/resources/protocols- and-sops/(Bench Protocol v5.6).

K562^{HER2} were washed three times with warmed PBS, incubated for 1-2 hours in serum free RPMI without phenol red and without glutamine, and resuspended in the same media at 0.8 million cells/mL. αHER2-eStcE, WA, StcE, or equal volume PBS were added to a final concentration of 1 nM, and the cells were incubated overnight at 37° C. Cells were spun down at 600×g for 5 minutes, and conditioned supernatant was collected and treated with protease inhibitors (complete, EDTA-free Protease Inhibitor Cocktail) and 10 mM EDTA. Conditioned supernatant was clarified by centrifugation at 1000×g for 5 minutes at 4° C. Trichloroacetic acid was added to a final concentration of 15% (v/v), and the mixture sat on ice for 3-4 hours. Precipitated proteins were washed three times by repeated pelleting by centrifugation at 9000×g for 15 minutes at 4° C., decanting of the supernatant, and resuspension of the pellets in −20° C. 100% acetone. After the final spin, the supernatant was decanted, and the pellets were frozen overnight.

Pellets were resuspended in 100 ul of 6M guanidine hydrochloride, and protein amounts were determined by BCA protein assay kit (Thermo Fisher Scientific). 125 μg of total protein material was used for each sample, and samples were adjusted to a total volume of 175 μL with water. Samples were then adjusted to 100 mM HEPES before adding freshly prepared TCEP to a final concentration of 10 mM and incubation at 37° C. for 30 minutes. Freshly prepared N-ethylmaleimide (NEM, adjusted to pH 6) was added to a final concentration of 15 mM, and samples were incubated for 10 minutes in the dark at room temperature. 50 μL of 100 mM TCEP was added and samples were vortexed to quench NEM before samples were put on ice. Proteins were precipitated using acetone, where a 4-fold volume of ice-cold acetone was added to each sample in an acetone-compatible tube. Samples were vortexed and incubated at −80° C. overnight before centrifuging at 18,000×g for 10 minutes. Supernatant was decanted with care taken not to disturb the protein pellet, and pellets were air dried for 15 minutes in an uncapped tube. Samples were then labeled with 16-plex Tandem Mass Tags (TMT, Thermo Fisher Scientific) according to the labeling scheme in Table 7, with distinct differences from manufacturer protocols because of labeling at protein rather than peptide level. Samples were resuspended in 110 μL 100 mM TEAB, and TMT labels (0.8 mg each) were dissolved in 110 μL DMSO. Samples were vortexed prior to a 1 hour incubation in the dark at 25° C. Following incubation, samples were combined into a single tube, and proteins were precipitating using the same acetone precipitation described above with overnight incubation. Following the 15 minute airdry, the pellet was resuspended in 50 μL 6M guanidine hydrochloride and then diluted ten-fold with 100 mM HEPES, pH 8.0. Trypsin was added at a protease: protein ratio of 1:100, with gentle mixing with a pipette prior to overnight incubation at 37° C. Negative selection for N-terminal peptides was performed using 45 mg/mL HPG-ALDII obtained from the Overall Lab (29 mg aliquot, Lot #002121800521). The HPG-ALDII polymer was thawed at room temperature and added to the digested sample at a polymer: peptide ratio of 6:1. Then sodium cyanoborohydride was immediately added to a final concentration of 20 mM and the sample was gently mixed, the pH was confirmed to be ˜6-7, and the sample was incubated overnight at 37° C. Sample recovery was performed the following day, with all spins at 12,000×g for 10 minutes at room temperature. Tris pH 6.8 was added to a final concentration of 100 mM, pH 6-7 was verified, and the sample was incubated for 30 minutes at 37° C. A 10-kDA molecular weight cutoff Amicon column was pre-washed with 400 μL 100 mM NaOH and 400 μL water, and flow-through (FT) was discarded. The peptide-polymer mixture was spun through the column and FT was collected into a clean tube labeled TAILS sample 1. The filter was then washed by spinning 400 μL water through, this FT was added to TAILS sample 1, and then the filter was thoroughly washed with 100 μL water, which rids the filter of the very hydrophilic polymer. The filter was repositioned upside down in a new tube labeled TAILS sample 2 with a quick spin to increase the yield of hydrophobic peptides. TAILS sample 1 and sample 2 were lyophilized before they were desalted using 10 mg/l mL Strata-X columns (Phenomenex). Briefly, columns were wet with 1 mL acetonitrile followed by equilibration with 1 mL 0.2% formic acid (FA) in water. Samples were resuspended in 500 μL 0.2% FA in water and were loaded on the column, followed by a wash with 1 mL 0.2% FA. Peptides were eluted with 400 μL 0.2% FA, 80% acetonitrile, dried via lyophilization, then resuspended in 0.2% FA in water prior to MS analysis.

TABLE 7
Sample IDs and TMT labeling for TAILS MS.

| Channel | Sample | Channel | Sample |
|---|---|---|---|
| 126C | PBS 1 | 127N | StcE 1 |
| 127C | PBS 2 | 128N | StcE 2 |
| 128C | PBS 3 | 129N | StcE 3 |
| 129C | PBS 4 | 130N | StcE 4 |
| 130C | eStcE 1 | 131N | αHER2-eStcE 1 |
| 131C | eStcE 2 | 132N | αHER2-eStcE 2 |
| 132C | eStcE 3 | 133N | αHER2-eStcE 3 |
| 133C | eStcE 4 | 134N | αHER2-eStcE 4 |

TAILS mass spectrometry LC-MS/MS. Both TAILS sample 1 and sample 2 were analyzed using 90-minute LC-MS/MS acquisitions, and TAILS sample 1 was analyzed with an additional 240-minute LC-MS/MS acquisition. Peptide mixtures were separated over a 25 cm EasySpray reversed phase LC column (75 μm inner diameter packed with 2 μm, 100 Å, PepMap C18 particles, Thermo Fisher Scientific). The mobile phases (A: water with 0.2% formic acid and B: acetonitrile with 0.2% formic acid) were driven and controlled by a Dionex Ultimate 3000 RPLC nano system (Thermo Fisher Scientific). An integrated loading pump was used to load peptides onto a trap column (Acclaim PepMap 100 C18, 5 μm particles, 20 mm length, Thermo Fisher Scientific) at 5 μL/min, which was put in line with the analytical column 5.5 minutes into the gradient. Gradient elution was performed at 300 nL/min for all analyses. For the 90-minute acquisitions, the gradient was held at 0% B for the first 6 min of the analysis, followed by an increase from 0% to 5% B from 6 to 6.5 min, an increase from 5% to 22% B from 6.5 to 66.5 min, an increase from 22% to 90% B from 66.5 to 70 min, isocratic flow at 90% B from 70 to 75 min, and a re-equilibration at 0% B for 15 min. For the 240-minute acquisitions, the gradient was held at 0% B for the first 6 min of the analysis, followed by an increase from 0% to 5% B from 6 to 6.5 min, an increase from 5% to 25% B from 6.5 to 200 min, an increase from 25% to 90% B from 200 to 218 min, isocratic flow at 90% B from 218 to 224 min, and a re-equilibration at 0% B for 16 min. For all methods, eluted peptides were analyzed on an Orbitrap Fusion Tribrid MS system (Thermo Fisher Scientific). Precursors were ionized using an EASY-Spray ionization source (Thermo Fisher Scientific) source held at +2.2 kV compared to ground, and the column was held at 40° C. The inlet capillary temperature was held at 275° C. Survey scans of peptide precursors were collected in the Orbitrap from 350-1500 Th with an AGC target of 250% (1,000,000 charges), a maximum injection time of 50 ms, and a resolution of 60,000 at 200 m/z. For 90-minute analyses, monoisotopic precursor selection was enabled for peptide isotopic distributions, precursors of z=2-5 were selected for data-dependent MS/MS scans for 2 seconds of cycle time, and dynamic exclusion was set to 30 seconds with a +10 ppm window set around the precursor monoisotope. An isolation window of 1 Th was used to select precursor ions with the quadrupole. MS/MS scans were collected using HCD at 30 normalized collision energy (nce) with an AGC target of 200% (100,000 charges) and a maximum injection time of 118 ms. Mass analysis was performed in the Orbitrap with a resolution of 60,000 with a first mass set at 120 m/z. All sets were the same for 240-minute analyses, with the exception of a 3 second cycle time and a 60 second dynamic exclusion time.

TAILS mass spectrometry data analysis. All raw data files were processed in batch using MaxQuant⁸, where the Andromeda search engine⁹ was used to search the entire human proteome downloaded from Uniprot (reviewed, 20428 entries). Cleavage specificity was set to “semi-specific free N-terminus” with ArgC specificity. The NEM modification of cysteine had to be created, with an addition of C6H702N (125.0478 Da) 10, that was as a fixed modification, while, oxidation methionine was set as a variable modification, with 5 maximum modifications per peptide. The experiment type was set to Reporter ion MS2 with 16-plex TMT modifications selected (user defined modifications added for both Lys and N-terminal labeling). The reporter ion mass tolerance was set to 0.003 Da and the minimum reporter PIF score was set to 0.75. Defaults were used for the remaining settings, including PSM and protein FDR thresholds of 0.01 and 20 ppm, 4.5 ppm, and 20 ppm for first search MS1 tolerance, main search MS1 tolerance, and MS2 product ion tolerance, respectively. “Match between runs” and “second peptide” options were not enabled. Quantified peptides were then processed in Perseus¹¹. Contaminants and reverse hits were removed, and signal in all relevant TMT channels of at least one condition was required to retain protein identifications.

The four proteins specifically degraded by StcE as compared to PBS (CD99L2, TNFRSF1B, CD55, CD46) were manually searched for regions with a high density of predicted mucin-type o-glycosylation using NetOGlyc-4.012. To account for phosphorylated residues incorrectly predicted as glycosylated residues, phosphorylation was annotated using PhosphoSitePlus¹³.

Generation of HER2 stable lines. pMXs-HER2 vector was generated by cloning the HER2+ coding sequence (Addgene) into the pMXs-FLAG backbone using In-Fusion HD Cloning Plus (Takara Bio). 1.5×10⁶ HEK-293 Ts were seeded into 6 cm dishes in 5 mL of complete media. 28 h later, 1 μg of pMXs-HER2 was mixed with 900 ng of retrovirus pol/gag, 150 ng of VSVg DNA, 130 μL of DMEM, and 6 μL of 1 mg/mL polyethylenimine (PEI). The mixture was incubated for 20 min at room temperature and added to HEK-293T cells dropwise. 18 h later, the culture media was replaced with 5 mL of DMEM supplemented with 30% heat inactivated FBS and 1% P/S. 30 h later, the media was collected and spun at 1000 rpm for 5 min. The clarified supernatant was stored at −80° C. prior to infection. To establish stably expressing cell lines, 1.5×10⁶ cells were seeded in 6-well plates in 2.8 mL of complete media. Polybrene was added at 10 μg/mL and cells were infected with 200 μL of virus-containing media. Plates were spun at 2200 rpm for 45 min and incubated at 37° C., 5% CO₂. 12-24 h later, cells were lifted with trypsin and plated in 10 cm dishes with 10 μg/mL blasticidin S (Thermo Fisher Scientific). Stable cell lines were tested for expression of HER2 by flow cytometry.

HER2 flow cytometry. Log-phase cells were aliquoted into a V-bottom 96-well plate at 5×10⁵ cells/well. Cells were washed twice with cold FACS buffer with 2 mM EDTA, once with cold FACS buffer, and stained with Alexa Fluor 488 anti-human CD340 (erbB2/HER-2) antibody (24D2 clone) in FACS buffer containing 0.1% benzonase on ice protected from light. Cells were washed three times with FACS buffer with 2 mM EDTA and stained with 30 nM Sytox Green for 10 min at 4° C. prior to analysis using a BD Accuri C6 plus.

K562 mixed cell CD43 cleavage assay. 2.5×10⁵ K562 and K562^{HER2} cells were allocated per well to a 96-well ultra-low attachment round bottom plate in 150 μL of complete media. 50 μL of mucinases in PBS were added to wells and the plate was incubated (overnight unless stated otherwise) at 37° C. Cells were washed twice with cold FACS buffer with 2 mM EDTA, once with cold FACS buffer, and stained with Alexa Fluor 488 anti-human CD340 (erbB2/HER-2) antibody (24D2 clone) (BioLegend) and Alexa Fluor 647 CD43/sialophorin antibody (MEM⁻⁵⁹ clone) (Novus Biologicals) in FACS buffer supplemented with 0.1% benzonase on ice protected from light. Cells were washed three times with cold FACS buffer with 2 mM EDTA and stained with 1 μM Sytox AADvanced (Thermo Fisher Scientific) or 1 μM Sytox Blue in FACS buffer with 2 mM EDTA for 5 min on ice prior to analysis using a BD Accuri C6 plus or MACSQuant Analyzer 10 Flow Cytometer (Miltenyi Biotec). Samples were compensated using single-stained controls in FlowJo v. 10.0. Unstained and PBS-treated samples were used to define 0% and 100% cell surface CD43, respectively, and each sample was normalized to percent CD43 within each replicate. Using GraphPad Prism 9, replicates were fitted to inhibitor concentration vs normalized response.

For the experiment validating αIgG1-eStcE cutting in a mixed cell assay, K562^{HER2} cells were stained with 1.25 UM CellTrace Violet (Thermo Fisher Scientific) in RPMI at 37° C. for 20 min and quenched with complete media for 5 min. 2.5×10⁵ K562 and CellTrace Violet-stained K562HER2 cells were mixed and treated as above with αHER2-eStcE or αIgG1-eStcE and 10 μg/mL Mouse IgG1 anti-human CD340 (erbB2/HER-2) antibody (24D2 clone) (BioLegend). Cells were stained and analyzed as above, except no Alexa Fluor 488 anti-HER2 was used.

MCF10A^{MUC1} mixed cell MUC1 cleavage assay. MUC1ΔCT was induced with 1 μg/ml doxycycline for 24 h. HER2⁺ cells were stained with 5 UM Molecular Probes CellTracker Green CMFDA Dye (ThermoFisher Scientific) in PBS at 37° C. for 30 min and washed twice with warmed PBS. 2.5×10⁵ MCF10^{AMUC1, HER2} and 2.5×10⁵ MFC10A^{MUC1} (matched for induction or not with doxycycline) were added to each well of a low adhesion U-bottom 96-well plate in 150 μL of complete media. 50 μL of mucinases in PBS were added to wells and the plate was incubated overnight at 37° C., 5% CO₂. Cells were washed twice with cold FACS buffer with 2 mM EDTA, once with cold FACS buffer, and stained with MUC1 mouse mAb (clone VU4H5) (Cell Signaling Technology) in FACS buffer supplemented with 0.1% benzonase on ice for 30 min. Cells were washed three times with cold FACS buffer with 2 mM EDTA, stained with Alexa Fluor 647 Affinipure Goat Anti-Mouse IgG (Jackson ImmunoResearch) for 30 min on ice, washed three times with cold FACS buffer with 2 mM EDTA, and stained with 1 μM Sytox AADvanced (ThermoFisher Scientific) in FACS buffer with 2 mM EDTA for 5 min on ice prior to analysis using a BD Accuri C6 plus.

Kinetics of αHER2-eStcE binding and cutting. At different time points, 150 μL of complete media containing 5×10⁵ K562^{HER2} and 50 μL of 400 nM αHER2, αHER2-eStcE, or StcE in PBS were added to a 96-well ultra-low attachment round bottom plate and incubated at 37° C. At the end of the incubation (times reported are total incubation length, and incubation stopped for all time points simultaneously), the cells were washed twice with cold FACS buffer, stained with FITC anti-His antibody (clone GG11-8F3.5.1) (Miltenyi Biotec) and Alexa Fluor 647 CD43/sialophorin antibody (MEM⁻⁵⁹ clone) (Novus Biologicals) for 30 min in FACS buffer supplemented with 0.1% benzonase on ice protected from light. Cells were washed twice with cold FACS buffer with 2 mM EDTA and stained with 1 μM Sytox Blue in FACS buffer with 2 mM EDTA for 5 min on ice. At least 20,000 live single cells were analyzed using a MACSQuant Analyzer 10 Flow Cytometer (Miltenyi Biotec). At each time point, unstained and PBS-treated samples were used to define 0% and 100% cell surface CD43, respectively, and each sample was normalized to percent CD43 within each replicate. Using GraphPad Prism 9, replicates were fitted to inhibitor concentration vs normalized response.

αIgG1-eStcE binding to K562HER2. αIgG1-eStcE and αHER2-eStcE were labeled with Alexa Fluor 647 NHS Ester (Thermo Fisher Scientific) following manufacturer's instructions and mixed with unlabeled protein for a final consistent dye: ratio of 0.819. K562^{HER2} cells were stained with 1.25 μM CellTrace Violet as above. 2.5×10⁵ K562 and CellTrace Violet-stained K562^{HER2} cells were added to each well of a V-bottom 96-well plate and washed three times with cold FACS buffer. Cells were stained for 30 min with 5 μg/mL Mouse IgG1 anti-human CD340 (erbB2/HER-2) antibody (24D2 clone) (BioLegend) or left unstained in FACS buffer and 0.1% benzonase for 30 min on ice. Cells were washed three time with FACS buffer with 2 mM EDTA, stained with indicated concentrations of AF647-labeled αIgG1-eStcE or AF647-labeled αHER2-eStcE for 30 min on ice, washed three times with FACS buffer with 2 mM EDTA, and stained with 30 nM Sytox Green (Thermo Fisher Scientific) in FACS buffer with 2 mM EDTA for at least 5 min. Approximately 40,000 live single cells were analyzed using a MACSQuant Analyzer 10 Flow Cytometer (Miltenyi Biotec). Within each sample, binding was normalized to the greatest median fluorescence intensity (MFI) per conjugate, with 0 being defined as the MFI for the PBS-treated sample. To mitigate the hook effect, only concentrations with greater than 85% normalized binding past the maximum signal were included. In GraphPad Prism 9, each replicate was fitted to agonist concentration vs response with the lower limit restricted to 0 and log₁₀ (EC₅₀) was reported.

Selection and validation of primary antibodies for αIgG1-eStcE cutting. Cell surface K562^{HER2} targets were identified from the published K562 mucinome and selected if there were well-validated commercial mouse IgG1 antibodies. Targets were identified as mucins (positive mucinome enrichment score and classified as a mucin), mucin-associated (positive mucinome enrichment score but not classified as a mucin), or non-mucin associated (negative mucinome enrichment score and not classified as a mucin). K562^{HER2} cells were treated with 100 nM StcE or PBS for 1 h at 37° C. and aliquoted at 2.5×10⁵ cells per well into a 96-well V-bottom plate. Cells were washed twice with cold FACS buffer and stained with 1.25-20 μg/mL of each antibody in FACS buffer containing 0.1% benzonase for 30 min on ice. Cells were washed twice with FACS buffer and stained with 20 μg/mL Alexa Fluor 647 Goat anti-Mouse IgG1 (Invitrogen) in FACS buffer for 30 min on ice protected from light. Cells were washed twice with cold FACS buffer containing 2 mM EDTA, stained with 30 nM Sytox Green for 10 min, and analyzed using a MACSQuant Analyzer 10 Flow Cytometer (Miltenyi Biotec). For each antibody, the maximum median fluorescence intensity value achieved at any concentration was reported, and minimum concentration of antibody that achieved approximately 95% staining was used for CD43 cutting assays to minimize oversaturation of the primary antibody. The isotype control did not bind at any concentration, so 20 μg/mL was used in the cutting experiments to match the highest concentration used for any antibody (Table 8).

TABLE 8
Primary antibodies used with αIgG1-eStcE.

| | | | | | Concentration | |
|---|---|---|---|---|---|---|
| | | | | | of antibody | Maximum MFI |
| | Antibody | | | Mucinome | using in cutting | observed in |
| Target | clone | | | enrichment | experiment | binding assay |
| name | number | Vendor | Mucin? | score | (μg/mL) | (AU) |
| MUC1 | 214D4 | EMD Millipore | Yes | 9.41 | 5 | 327.8 |
| CD43 | CD43-10G7 | BioLegend | Yes | 8.52 | 10 | 160.4 |
| CD30 | BY88 | BioLegend | Yes | 7.18 | 5 | 9.8 |
| CD236 | QA20A41 | BioLegend | No | 8.48 | 5 | 188.5 |
| CD46 | TRA-2-10 | BioLegend | No | 7.26 | 2.5 | 64.9 |
| HER2 | 24D2 | BioLegend | No | — | 2.5 | 310.6 |
| CD98 | MEM-108 | BioLegend | No | −4.49 | 10 | 234 |
| Annexin | 74/3 | BioLegend | No | −7.44 | 20 | 1.3 |
| A1 (ANXA1) | | | | | | |
| CD29 | P5D2 | BioLegend | No | −4.17 | 2.5 | 23.7 |
| Isotype | MOPC-21 | BioLegend | | | 20 | 0.1 |

αIgG1-eStcE cutting CD43 on K562HER2. 5×10⁵ K562^{HER2} cells were allocated per well to a 96-well ultra-low attachment round bottom plate in 150 μL of complete media. 50 μL of mucinases or indicated concentration of primary antibody (Table 8) and αIgG1-eStcE in PBS were added to wells and the plate was incubated for 4 h at 37° C. Cells were washed twice with cold FACS buffer and stained with Alexa Fluor 647 CD43/sialophorin antibody (MEM⁻⁵⁹ clone) (Novus Biologicals) in FACS buffer supplemented with 0.1% benzonase on ice protected from light. Cells were washed twice with cold FACS buffer with 2 mM EDTA and stained with 30 nM Sytox Green in FACS buffer with 2 mM EDTA for 5 min on ice. At least 20,000 live single cells were analyzed on a MACSQuant Analyzer 10 Flow Cytometer (Miltenyi Biotec). Unstained and PBS-treated samples were used to define 0% and 100% cell surface CD43, respectively, and each sample was normalized to percent CD43 within each replicate. For the CD43 primary antibody sample, 100% cell surface CD43 was defined using a sample treated with the anti-CD43 primary and 0 nM αIgG1-eStcE. Using GraphPad Prism 9, replicates were fitted to inhibitor concentration vs normalized response to generate EC₅₀ values of cutting.

Macrophage phagocytosis assay. MCF7 and MCF7^{HER2} cells were lifted with enzyme-free cell dissociation buffer and resuspended in PBS. MCF7 and MCF7^{HER2} cells were incubated in 5 μg/mL Alexa Fluor 546 C5 maleimide (Invitrogen) and 5 μg/mL Alexa Fluor 647 C2 maleimide (Invitrogen), respectively, for 20 min rotating at room temperature. Cells were resuspended in 5 mM N-ethyl-maleimide (Sigma Aldrich) in PBS and incubated for 20 min rotating at room temperature. Cells were resuspended in PBS and treated with PBS, 5 nM endotoxin-free StcE, or 100 nM αHER2-eStcE as appropriate for 2 h at 37° C. After 1 h, InVivoMAb anti-mouse/human/rat CD47 (clone MIAP410) (BioXCell) was added to a final concentration of 20 μg/mL. The media for the macrophages was replaced with serum-free RPMI and appropriate wells were treated with 10 μM cytochalasin D (Invitrogen). MCF7 and MCF7^{HER2} cells were washed twice with PBS and resuspended in 100 μL of serum-free RPMI. Macrophage media was replaced with 200 μL of serum-free RPMI. MCF7 and MCF7^{HER2} cells of the same treatment group were mixed and added to the appropriate macrophage well and incubated for 30 min at 37° C. After incubation, macrophages were gently washed five times with cold PBS. Cells were fixed with 4% paraformaldehyde in PBS for 15 min at room temperature. Cells were then rinsed with PBS and permeabilized with 0.5% Triton-X-100 in PBS for 10 min. Cells were subsequently rinsed with PBS and blocked with 2% BSA in PBS for 20 min. Cells were then stained with Alexa Fluor 488 phalloidin (Invitrogen) (1:2000) and 7.5 μM DAPI in PBS for 20 min at room temperature. Cells were then washed three times with PBS and stored in PBS at 4° C. until imaging using a Nikon A1R confocal microscope. Phagocytosis binding indices were calculated as the surface area of target cells divided by the number of macrophages in the field of view. Surface area and number of macrophages were calculated using the imaging software Imaris. Normalized binding indices were calculated relative to the binding index of the PBS treatment condition of the appropriate biological replicate. Three biological replicates were done with macrophages isolated from three different human donors.

In vivo toxicity studies. 10-week-old C57BL/6 mice (bred in-house) were injected with PBS or StcE (0.15 mg/kg or 15 mg/kg) via intravenous injection. 9-week-old female BALB/cJ mice (Jackson Labs) were injected with PBS or 10 mg/kg αHER2-eStcE via intraperitoneal injection. Mice were submitted to the Animal Diagnostic Lab at Stanford University for necropsy and complete blood count (CBC) analyses 3 h post injection.

In vivo biodistribution and mucin degradation Western blots. Experiments involving animals were approved under Stanford APLAC protocol no. 31511. 12-week-old male BALB/cJ mice (Jackson Labs) were injected with PBS, 10 mg/kg StcE, or 0.25 mg/kg IRdye 800CW-labeled StcE via intraperitoneal injection. Liver, spleen, lung, and plasma (submandibular bleed) were collected at the indicated times post injection. Tissues were lysed using a Bead Mill 24 Homogenizer (Fisher Scientific) in RIPA buffer (Thermo Fisher Scientific) supplemented with benzonase and complete Mini, EDTA-free Protease Inhibitor Cocktail Tablets (Sigma Aldrich). Plasma and tissue lysates (40-50 μg) were loaded onto a 4 to 12% Criterion XT Bis-Tris protein gel and run in XT-MOPS at 180 V for 1 h. Total protein was visualized with AcquaStain protein gel stain (Bulldog-Bio). For mucin Western blots, the gel was transferred to a 0.2-μm nitrocellulose membrane using the Trans-Blot Turbo Transfer System (Bio-Rad) at 2.5 A for 15 min. Total protein was quantified using REVERT stain (LI-COR Biosciences). The membrane was blocked with Carbo-free Blocking Solution (Vector Laboratories) supplemented with 0.1% v/v Tween-20 for 1 h at room temperature and then incubated with 10 μg/mL biotin-StcE^{E447D} in PBS-T (0.1% v/v Tween-20) at room temperature for 1 h. IRDye 800CW streptavidin (LI-COR Biosciences) was used according to manufacturer recommendations. Blots were imaged using an Odyssey CLx Near-Infrared Fluorescence Imaging System (LI-COR Biosciences). For cell line Western blots, anti-MUC1 (clone 214D4) (EMD Millipore) and anti-MUC16 (clone X75) (Abcam) were used according to manufacturer instructions.

8-week-old female BALB/cJ mice (Jackson Labs) were injected with PBS or IRdye 680RD-αHER2-eStcE at 0.25, 0.5, 1, 2, 5, and 10 mg/kg via retro-orbital injection. Plasma (tail bleed) was collected at t=1, 3, 6, 20, and 48 h post injection. Liver, kidney, spleen, lung, and heart tissues for the mice injected with 10 mg/kg αHER2-eStcE were collected at t=20 and 48 h post injection. Plasma samples (1 μL) were loaded onto a 4 to 12% Criterion XT Bis-Tris protein gel and run in XT-MOPS at 180 V for 1 h. Tissues were lysed as described above and lysates (30 μg) were loaded onto a 4 to 12% Criterion XT Bis-Tris protein gel and run in XT-MOPS at 180 V for 1 h. Total protein was visualized with AcquaStain protein gel stain (Bulldog-Bio). Gels were imaged using an Odyssey CLx Near-Infrared Fluorescence Imaging System. 7-week-old female BALB/cJ mice (Jackson Labs) were injected with PBS, 5 mg/kg StcE, or 5 mg/kg αHER2-eStcE via retro-orbital injection. Liver, spleen, lung, and plasma (submandibular bleed) were collected 4 h post injection. Mucin Western blot was performed as described above.

In vivo intestinal permeability assay. Experiments involving animals were approved under Stanford APLAC protocol no. 31511. 7-week-old female BALB/cJ mice (Jackson Labs) were injected with PBS or 10 mg/kg αHER2-eStcE via intraperitoneal injection every other day (days 1, 3, 5, 7) for a total of 4 doses. On day 8, mice were fasted for 4 h in cages without food or bedding. After fasting, blood was collected via tail vein nick and 15% v/v acid-citrate-dextrose solution (Sigma) was added. Mice were given an oral gavage (150 μL) of 80 mg/mL FITC-dextran (4 kDa) dissolved in PBS. After 4 h, blood collection was repeated. All blood samples were spun down at 5000 rpm for 10 min to isolate plasma. Plasma samples were diluted 1:10 in 100 μL of PBS and transferred to a black opaque-bottom 96-well plate. A serial dilution of FITC-dextran (0.2 to 12.5 μg/mL range) in PBS with 10% v/v mouse plasma was included for comparison. Fluorescence signal (excitation: 485 nm, emission: 540 nm) was measured using a SpectraMax i3× microplate reader.

4T07^{MUC1,HER2} mouse model. Experiments involving animals were approved under UCSF Institutional Animal Care and Use Program (IACUC) protocol no. AN179766. 4T07 cells expressing a cytoplasmic truncation of MUC1 (MUC1ΔCT, also referred to as MUC1 ectodomain) were used to limit any possible cytoplasmic signaling¹. 1×10⁶ 4T07^{HER2} breast cancer cells expressing mApple luciferase and doxycycline-inducible MUC1ΔCT were seeded in the lungs of female, syngeneic 8-week-old BALB/cJ mice by intravenous injection (tail vein). Cells were stimulated with 2 mg/ml doxycycline for 24 h to induce MUC1ΔCT expression prior to injection. Competent cell seeding of the lungs was assessed by bioluminescent imaging (BLI; IVIS In Vivo Imaging System) following intraperitoneal injection of D-Luciferin (150 mg/kg) within 30 min of cell injection. Mice were given systemic treatments of PBS or 10 mg/kg αHER2-eStcE by intravenous injection every 2 days for a total of 7 doses. Tumor cell lung burden was assessed over time by additional BLI measurements of mice. BLI for independent images was calculated from total bioluminescence flux for the chest region of each mouse. At 15 days, animals were sacrificed and lungs were harvested. Whole animal and gross lung weights were recorded, as well as the number of detected lung surface lesions. Lungs were then formalin fixed and processed for paraffin embedding and the average lung lesion area for each animal was determined from H&E-stained tissue sections. In a separate experiment, 4T07^{MUC1,HER2} cells were seeded as above and mice were immediately treated with one intravenous dose of PBS, 10 mg/kg αHER2-eStcE, or an equimolar dose of αHER2-eStcE^{E447D} or αGFP-eStcE (day 0). Two additional intravenous doses of each treatment were completed on day 2 and day 4. Mice received a diet of gamma irradiated doxycycline-chow (Bio-Serv Cat. #: 55829; 625 mg/kg) for the duration of the experiment to maintain MUC1ΔCT expression. Mouse lung metastatic burden was monitored by bioluminescence imaging (BLI) on days 0, 3, 5 and 8.

EMT6^{HER2} mouse model. BALB/c mice were obtained from Janvier Laboratories and bred in-house at the University Hospital Basel, Switzerland. All mouse experiments were approved by the local ethics committee (Approval 2370 and 3036, Basel Stadt, Switzerland). Animals were housed under specific pathogen-free conditions. For tumor growth experiments, 8-12-week-old females were used. 1×10⁶ EMT6^{HER2} cells were injected into the right mammary fat pad of female BALB/c mice. For efficacy studies, four I.P. doses of PBS, 10 mg/kg αHER2-eStcE, or an equimolar quantity (2.8 nmol) of αHER2, αHER2-eStcE^{E447D}, or αGFP-eStcE were administered every 2 days for a total of 4 doses once the tumor size reached an average size of 80-100 mm³. For analysis of the tumor infiltration by flow cytometry, two I.P. doses of PBS, 10 mg/kg αHER2-eStcE or an equimolar quantity of αHER2 were administered every 2 days for a total of 2 doses once the tumor size reached an average size of 80-100 mm³. Perpendicular tumor diameters were measured by caliper and tumor volume calculated according to the following formula: tumor volume (mm³)=(d x D)/2, where d and D are the shortest and longest diameters of the tumor (in millimeters), respectively. Mice were killed once tumor size reached approximately 1500 mm³ or when the mice developed ulcerated tumors that required euthanasia and the animals excluded from further analysis.

Quantitative histological analysis of lung tissues for the 4T07^{MUC1,HER2} mouse model. Analysis of IHC and H&E-stained lung tissue sections was performed using ImageJ and QuPath software, respectively. Specifically, an IHC profiler ImageJ plugin was used to quantify percent positive DAB staining area of lung metastases with selection for either cytoplasmic (phospho-FAK, phospho-(Ser/Thr) Akt substrates) or nuclear (Cyclin D1) staining. Reported values correspond to the sum of high positive and positive DAB signal. To assess metastatic lesion area throughout the depth of mouse lung tissues, lungs were sectioned as two steps spaced 40 μm apart, with 15 sequential sections of 5 μm cut at each step. The top and bottom sections from each step were then stained with H&E (75 μm apart) and slides were scanned using a ZEISS Axio Scan.Z1 digital slide scanner equipped with CMOS and color cameras and 10×, 20× and 40× objectives. Percent area of lung metastasis for each section was determined in Qupath using the polygon tool to trace and annotate lung lesion area compared to whole tissue section area. The average values of two lung sections from each animal are presented.

Flow cytometry analysis of tumor infiltrating immune cells. Thawed single cell suspensions were stained with antibodies noted above in Table 4 and analyzed on Y instrument. Live single cells were gated for different immune subsets as diagrammed in FIG. 26. For t-SNE analysis, live single CD45+ cells were randomly down sampled using the FlowJo DownSample v3.3.1 plugin. tSNE analysis was performed using the FlowJo t-SNE plugin once on the concatenated files using all compensated fluorophores (default settings: learning configuration=opt-SNE, iterations =1000, perplexity=30, learning rate=6996, KNN algorithm=exact (vantage point tree), gradient algorithm=Barnes-Hut) on ˜100,000 total cells evenly disturbed between treatment groups and evenly distributed between biological replicates in each treatment group. For analysis of activation states, gates for positive staining were defined using unstained samples and were kept consistent for all immune subsets (FIG. 26b).

Analysis of in vivo mucin cleavage in EMT6^{HER2} mouse model. For the preparation of single cell suspensions, tumors were collected, surgical specimens were mechanically dissociated and subsequently digested using Accutase (PAA Laboratories), collagenase IV (Worthington), hyaluronidase (Sigma) and DNase type IV (Sigma) for 1 h at 37° C. under constant agitation. Cell suspensions were filtered through a 70 μm mesh, 10 μL of CountBright™ Plus absolute counting beads (Invitrogen) were added, and samples were frozen at −80° C. Single cell suspensions processed and frozen above were briefly thawed in a 37° C. water bath and immediately placed on ice. Cells were washed once, counted, and 1.9-3×10⁶ cells were processed per biological sample. Cells were washed once with cold FACS buffer, treated with Mouse BD Fc Block in cold FACS buffer for 5 min on ice, and immediately stained with Brilliant Violet 421 CD45 antibody (30-F11), Alexa Fluor 488HER2 antibody (24D2), and Alexa Fluor StcE^{E447D} (5 μg/mL) in FACS buffer with 1:1000 benzonase for 30 min on ice protected from light. UltraComp eBeads Plus Compensation Beads (Thermo Fisher Scientific) were stained in parallel for antibody single color controls following manufacturer's recommendations. Cells were washed once with cold PBS and stained with 1:1000 GloCell Fixable Viability Dye Violet 510 (StemCell Technologies) in PBS for 30 min on ice protected from light. ArC Amine Reactive Compensation Beads (Thermo Fisher Scientific) were stained in parallel for viability single color controls following manufacturer's recommendations. Cells were washed once with cold FACS buffer with 2 mM EDTA, resuspended in cold FACS buffer with 2 mM EDTA, and analyzed using MACSQuant Analyzer 10 Flow Cytometer (Milyteni Biotec). EMT6^{HER2} and immune cells were gated from live single cells as shown in Fig. S12e using fluorescence minus one controls. Correct gating of these populations was confirmed with EMT6^{HER2} and commercial mouse PBMCS (IQ Biosciences) stained and analyzed in parallel with the tumor samples.

AlphaFold modeling. Protein sequences for StcE and αHER2-eStcE were used as inputs for ColabFold (https://colab.research.google.com/github/sokrypton/ColabFold/blob/main/AlphaFold².ipynb #scrollTo=kOblAo-xetgx) to obtain model structures¹⁴. The predicted structure for StcE aligned well (for previously reported StcE structure¹⁵ RMSD=0.2) with the X-ray structure determined by Yu et al.¹⁵. Molecular graphics were generated using PyMOL.

Flow cytometry analysis of tumor infiltrating immune cells. For the preparation of single cell suspensions, tumors were collected, surgical specimens were mechanically dissociated and subsequently digested using accutase (PAA Laboratories), collagenase IV (Worthington), hyaluronidase (Sigma) and DNase type IV (Sigma) for 1 h at 37° C. under constant agitation. Cell suspensions were filtered through a 70 μm mesh, 10 μl of CountBright™ Plus absolute counting beads (Invitrogen) were added, and samples were frozen (−80° C.) for further analysis of tumor-infiltrating immune cells by flow cytometry. Thawed samples were stained with antibodies shown above in Table 4 and analyzed on Cytek® Aurora instrument. Live single cells were gated for different immune subsets as diagrammed in Fig. S14a. For t-SNE analysis, live single CD45+ cells were randomly down sampled using the FlowJo DownSample v3.3.1 plugin. tSNE analysis was performed using the FlowJo t-SNE plugin once on the concatenated files using all compensated fluorophores (default settings: learning configuration=opt-SNE, iterations=1000, perplexity=30, learning rate=6996, KNN algorithm=exact (vantage point tree), gradient algorithm=Barnes-Hut) on ˜100,000 total cells evenly disturbed between treatment groups and evenly distributed between biological replicates in each treatment group. For analysis of activation states, gates for positive staining were defined using unstained samples and were kept consistent for all immune subsets (Fig. S14b).

### REFERENCES

1. Bakshani, C. R. et al. Evolutionary conservation of the antimicrobial function of mucus: a first defence against infection. NPJ Biofilms Microbiomes 4, 14 (2018).
2. Freeman, S. A. et al. Integrins Form an Expanding Diffusional Barrier that Coordinates Phagocytosis. Cell 164, 128-140 (2016).
3. Smith, B. A. H. & Bertozzi, C. R. The clinical impact of glycobiology: targeting selectins, Siglecs and mammalian glycans. Nat. Rev. Drug Discov. 20, 217-243 (2021).
4. Kufe, D. W. Functional targeting of the MUC1 oncogene in human cancers. Cancer Biol. Ther. 8, 1197-1203 (2009).
5. Xie, G.-D., Liu, Y.-R., Jiang, Y.-Z. & Shao, Z.-M. Epidemiology and survival outcomes of mucinous adenocarcinomas: A SEER population-based study. Sci. Rep. 8, 6117 (2018).
6. Dvela-Levitt, M. et al. Small Molecule Targets TMED9 and Promotes Lysosomal Degradation to Reverse Proteinopathy. Cell 178, 521-535.e²³ (2019).
7. Burslem, G. M. et al. The Advantages of Targeted Protein Degradation Over Inhibition: An RTK Case Study. Cell Chem. Biol. 25, 67-77.e³ (2018).
8. Blum, T. R. et al. Phage-assisted evolution of botulinum neurotoxin proteases with reprogrammed specificity. Science 371, 803-810 (2021).
9. Banik, S. M. et al. Lysosome-targeting chimaeras for degradation of extracellular proteins. Nature 584, 291-297 (2020).
10. Steentoft, C. et al. Precision mapping of the human O-GalNAc glycoproteome through SimpleCell technology. EMBO J. 32, 1478-1488 (2013).
11. Noach, I. et al. Recognition of protein-linked glycans as a determinant of peptidase activity. Proc. Natl. Acad. Sci. 201615141 (2017) doi: 10.1073/pnas.1615141114.
12. Malaker, S. A. et al. The mucin-selective protease StcE enables molecular and functional analysis of human cancer-associated mucins. Proc. Natl. Acad. Sci. U.S.A 116, 7278-7287 (2019).
13. Shon, D. J. et al. An enzymatic toolkit for selective proteolysis, detection, and visualization of mucin-domain glycoproteins. Proc. Natl. Acad. Sci. 117, 21299-21307 (2020).
14. Nason, R. et al. Display of the human mucinome with defined O-glycans by gene engineered cells. Nat. Commun. 12, 4070 (2021).
15. Shurer, C. R. et al. Genetically Encoded Toolbox for Glycocalyx Engineering: Tunable Control of Cell Adhesion, Survival, and Cancer Cell Behaviors. ACS Biomater. Sci. Eng. 4, 388-399 (2018).
16. Shurer, C. R. et al. Physical Principles of Membrane Shape Regulation by the Glycocalyx. Cell 177, 1757-1770.e²¹ (2019).
17. Wisnovsky, S. et al. Genome-wide CRISPR screens reveal a specific ligand for the glycan-binding immune checkpoint receptor Siglec-7. Proc. Natl. Acad. Sci. 118, (2021).
18. Jonckheere, N., Skrypek, N. & Van Seuningen, I. Mucins and tumor resistance to chemotherapeutic drugs. Biochim. Biophys. Acta BBA-Rev. Cancer 1846, 142-151 (2014).
19. Forcina, G. C., Conlon, M., Wells, A., Cao, J. Y. & Dixon, S. J. Systematic Quantification of Population Cell Death Kinetics in Mammalian Cells. Cell Syst. 4, 600-610.e⁶ (2017).
20. Dixon, S. J. et al. Ferroptosis: An Iron-Dependent Form of Nonapoptotic Cell Death. Cell 149, 1060-1072 (2012).
21. Yang, W. S. et al. Regulation of Ferroptotic Cancer Cell Death by GPX4. Cell 156, 317-331 (2014).
22. Egler, R. A., Ahuja, S. P. & Matloub, Y. L-asparaginase in the treatment of patients with acute lymphoblastic leukemia. J. Pharmacol. Pharmacother. 7, 62-71 (2016).
23. Drago, J. Z., Modi, S. & Chandarlapaty, S. Unlocking the potential of antibody-drug conjugates for cancer therapy. Nat. Rev. Clin. Oncol. 18, 327-344 (2021).
24. Xiao, H., Woods, E. C., Vukojicic, P. & Bertozzi, C. R. Precision glycocalyx editing as a strategy for cancer immunotherapy. Proc. Natl. Acad. Sci. 113, 10304-10309 (2016).
25. Gray, M. A. et al. Targeted glycan degradation potentiates the anticancer immune response in vivo. Nat. Chem. Biol. 16, 1376-1384 (2020).
26. Yu, A. C. Y., Worrall, L. J. & Strynadka, N. C. J. Structural Insight into the Bacterial Mucinase StcE Essential to Adhesion and Immune Evasion during Enterohemorrhagic E. coli Infection. Structure 20, 707-717 (2012).
27. Malaker, S. A. et al. The mucin-selective protease StcE enables molecular and functional analysis of human cancer-associated mucins. Proc. Natl. Acad. Sci. 116, 7278-7287 (2019).
28. Fc-fusion proteins: new developments and future perspectives. EMBO Mol. Med. 4, 1015-1028 (2012).
29. Pruszynski, M. et al. Targeting breast carcinoma with radioiodinated anti-HER2 Nanobody. Nucl. Med. Biol. 40, 52-59 (2013).
30. Kleifeld, O. et al. Isotopic labeling of terminal amines in complex samples identifies protein N-termini and protease cleavage products. Nat. Biotechnol. 28, 281-288 (2010).
31. Imbert, P. R. C. et al. An Acquired and Endogenous Glycocalyx Forms a Bidirectional “Don't Eat” and “Don't Eat Me” Barrier to Phagocytosis. Curr. Biol. 31, 77-89.e⁵ (2021).
32. Aslakson, C. J. & Miller, F. R. Selective Events in the Metastatic Process Defined by Analysis of the Sequential Dissemination of Subpopulations of a Mouse Mammary Tumor. Cancer Res. 52, 1399-1405 (1992).
33. Woods, E. C. et al. A bulky glycocalyx fosters metastasis formation by promoting G1 cell cycle progression. eLife 6, e²⁵⁷⁵² (2017).
34. Pimental, R. A., Julian, J., Gendler, S. J. & Carson, D. D. Synthesis and Intracellular Trafficking of Muc-1 and Mucins by Polarized Mouse Uterine Epithelial Cells*. J. Biol. Chem. 271, 28128-28137 (1996).
35. Rockwell, S. C., Kallman, R. F. & Fajardo, L. F. Characteristics of a Serially Transplanted Mouse Mammary Tumor and Its Tissue-Culture-Adapted Derivative. JNCI J. Natl. Cancer Inst. 49, 735-749 (1972).
36. Senter, P. D. et al. Anti-tumor effects of antibody-alkaline phosphatase conjugates in combination with etoposide phosphate. Proc. Natl. Acad. Sci. 85, 4842-4846 (1988).
37. Davis, B. G. et al. Selective Protein Degradation by Ligand-Targeted Enzymes: Towards the Creation of Catalytic Antagonists. ChemBioChem 4, 533-537 (2003).
38. Békés, M., Langley, D. R. & Crews, C. M. PROTAC targeted protein degraders: the past is prologue. Nat. Rev. Drug Discov. 1-20 (2022) doi: 10.1038/s41573-021-00371-6.
39. Ge, Y. et al. Target protein deglycosylation in living cells by a nanobody-fused split O-GlcNAcase. Nat. Chem. Biol. 17, 593-600 (2021).
40. Gerry, C. J. & Schreiber, S. L. Unifying principles of bifunctional, proximity-inducing small molecules. Nat. Chem. Biol. 16, 369-378 (2020).
41. Putten, J. P. M. van & Strijbis, K. Transmembrane Mucins: Signaling Receptors at the Intersection of Inflammation and Cancer. J. Innate Immun. 9, 281-299 (2017).
42. Delaveris, C. S., Webster, E. R., Banik, S. M., Boxer, S. G. & Bertozzi, C. R. Membrane-tethered mucin-like polypeptides sterically inhibit binding and slow fusion kinetics of influenza A virus. Proc. Natl. Acad. Sci. 117, 12643-12650 (2020).
43. Chatterjee, M., Putten, J. P. M. van & Strijbis, K. Defensive Properties of Mucin Glycoproteins during Respiratory Infections-Relevance for SARS-COV-2. mBio (2020) doi: 10.1128/mBio.⁰²³⁷⁴-20.
44. Kreda, S. M., Davis, C. W. & Rose, M. C. CFTR, Mucins, and Mucus Obstruction in Cystic Fibrosis. Cold Spring Harb. Perspect. Med. 2, a009589 (2012).
45. Bensing, B. A. et al. Recognition of specific sialoglycan structures by oral streptococci impacts the severity of endocardial infection. PLOS Pathog. 15, e¹⁰⁰⁷⁸⁹⁶ (2019).
46. Hansson, G. C. Mucins and the Microbiome. Annu. Rev. Biochem. 89, (2020).
47. Mirdita, M., Ovchinnikov, S. & Steinegger, M. ColabFold-Making protein folding accessible to all. 2021.08.15.456425 (2021) doi: 10.1101/2021.08.15.456425.
48. Malaker, S. A. et al. Revealing the human mucinome. Nat. Commun. 13, 3542 (2022).
49. Hornbeck, P. V. et al. PhosphoSitePlus, 2014: mutations, PTMs and recalibrations. Nucleic Acids Res. 43, D512-520 (2015).
50. Forcina, G. C. et al. Ferroptosis regulation by the NGLY1/NFE2L1 pathway. Proc. Natl. Acad. Sci. 119, e²¹¹⁸⁶⁴⁶¹¹⁹ (2022).
51. Pleiner, T., Bates, M. & Görlich, D. A toolbox of anti-mouse and anti-rabbit IgG secondary nanobodies. J. Cell Biol. 217, 1143-1154 (2017).
52. Kubala, M. H., Kovtun, O., Alexandrov, K. & Collins, B. M. Structural and thermodynamic analysis of the GFP: GFP-nanobody complex. Protein Sci. 19, 2389-2401 (2010).
53. Jumper, J. et al. Highly accurate protein structure prediction with AlphaFold. Nature 596, 583-589 (2021).
54. Weiner, S. J. et al. A new force field for molecular mechanical simulation of nucleic acids and proteins. J. Am. Chem. Soc. 106, 765-784 (1984).

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A mucin-active protease stably associated with a targeting moiety.

2. The mucin-active protease of claim 1, wherein the mucin-active protease cleaves at a glycan-peptide cleavage motif comprising: S/T *-X-S/T, S/T *-S/T, X-S/T*, S/T *-X, and/or S/T *-X-X-X-X, wherein * denotes glycosylation of the S or T residue and X is any amino acid residue.

3. The mucin-active protease of claim 1 or claim 2, wherein the mucin-active protease cleaves C1-INH, CADM1, CD43, CD44, CD45, CD68, CXCL1, EMCN, GHA1, GLPA, GLPC, GP1BA, HAVCR1, HEG, MADCAM1, MUC1, MUC2, MUC3A, MUC3B, MUC4, MUC5AC, MUC5B, MUC6, MUC7, MUC8, MUC9, MUC10, MUC11, MUC12, MUC13, MUC14, MUC15, MUC16, MUC17, MUC18, MUC19, MUC20, MUC21, MUC22, MUCL3, PARM1, PILRA, PODXL, PRG4, PSGL1, TIMD4, or a combination thereof.

4. The mucin-active protease of any one of claims 1 to 3, wherein the mucin-active protease is a eukaryotic mucin-active protease.

5. The mucin-active protease of any one of claims 1 to 3, wherein the mucin-active protease is a prokaryotic mucin-active protease.

6. The mucin-active protease of claim 5, wherein the mucin-active protease is a secreted protease of C1 esterase inhibitor (StcE) from Escherichia coli 0157: H7.

7. The mucin-active protease of claim 6, wherein the StcE comprises 70% or greater, 75% or greater, 80% or greater, 85% or greater, 90% or greater, 95% or greater, 96% or greater, 97% or greater, 98% or greater, 99% or greater, or 100% amino acid identity with the amino acid sequence set forth in SEQ ID NO:1, or a functional fragment thereof which retains mucin-active protease activity.

8. The mucin-active protease of claim 6 or claim 7, wherein the StcE comprises one or more deletions relative to the amino acid sequence set forth in SEQ ID NO:1.

9. The mucin-active protease of claim 8, wherein the one or more deletions comprises a deletion of all or a portion of the C domain.

10. The mucin-active protease of claim 8 or claim 9, wherein the one or more deletions comprises a deletion of all or a portion of the INS domain.

11. The mucin-active protease of any one of claims 6 to 10, wherein the StcE comprises one or more amino acid substitutions at or near the active site.

12. The mucin-active protease of claim 11, wherein the one or more amino acid substitutions comprise a substitution at W366, H367, Y457, or any combination thereof.

13. The mucin-active protease of claim 12, wherein the one or more amino acid substitutions comprise W366A, H367A, or both.

14. The mucin-active protease of any one of claims 6 to 13, wherein the StcE comprises a deletion of all or a portion of the C domain, a deletion of all or a portion of the INS domain, and a W366A substitution.

15. The mucin-active protease of any one of claims 1 to 3, wherein the mucin-active protease is Pic, ZmpB, ZmpC, BT4244, AM0627, AM0908, AM1514, SmEnhancin, VIBHAR2194, CpaA, ImpA, or OgpA.

16. The mucin-active protease of any one of claims 1 to 15, wherein the mucin-active protease is a mucin-selective protease.

17. The mucin-active protease of any one of claims 1 to 16, wherein the mucin-active protease selectively recognizes a joint glycopeptide epitope.

18. The mucin-active protease of any one of claims 1 to 17, wherein the targeting moiety is selected from the group consisting of: a polypeptide, a ligand, an aptamer, a nanoparticle, and a small molecule.

19. The mucin-active protease of claim 18, wherein the targeting moiety is a polypeptide.

20. The mucin-active protease of claim 19, wherein the targeting moiety comprises an antibody.

21. The mucin-active protease of claim 20, wherein the antibody is an IgG, single chain Fv (scFv), Fab, (Fab)₂, (scFv′)₂, or a single variable domain located on a heavy chain (VHH).

22. The mucin-active protease of claim 20, wherein the antibody is a VHH.

23. The mucin-active protease of any one of claims 1 to 22, wherein the targeting moiety specifically binds a cell surface molecule.

24. The mucin-active protease of any one of claims 1 to 23, wherein the targeting moiety specifically binds to a tumor antigen on the surface of the cancer cell.

25. The mucin-active protease of claim 24, wherein the tumor antigen is 5T4, AXL receptor tyrosine kinase (AXL), B-cell maturation antigen (BCMA), c-MET, C4.4a, carbonic anhydrase 6 (CA6), carbonic anhydrase 9 (CA9), Cadherin-6, CD19, CD20, CD22, CD25, CD27L, CD30, CD33, CD37, CD44, CD44v6, CD56, CD70, CD74, CD79b, CD123, CD138, carcinoembryonic antigen (CEA), cKit, Cripto protein, CS1, delta-like canonical Notch ligand 3 (DLL3), endothelin receptor type B (EDNRB), ephrin A4 (EFNA4), epidermal growth factor receptor (EGFR), EGFRvlll, ectonucleotide pyrophosphatase/phosphodiesterase 3 (ENPP3), EPH receptor A2 (EPHA2), fibroblast growth factor receptor 2 (FGFR2), fibroblast growth factor receptor 3 (FGFR3), FMS-like tyrosine kinase 3 (FLT3), folate receptor 1 (FOLR1), GD2 ganglioside, glycoprotein non-metastatic B (GPNMB), guanylate cyclase 2 C (GUCY2C), human epidermal growth factor receptor 2 (HER2), human epidermal growth factor receptor 3 (HER3), Integrin alpha, lysosomal-associated membrane protein 1 (LAMP-1), Lewis Y, LIV-1, leucine rich repeat containing 15 (LRRC15), mesothelin (MSLN), mucin 1 (MUC1), mucin 16 (MUC16), sodium-dependent phosphate transport protein 2B (NaPi2b), Nectin-4, NMB, NOTC_{H}3, p-cadherin (p-CAD), programmed cell death receptor ligand 1 (PD-L1), programmed cell death receptor ligand 2 (PD-L2), prostate-specific membrane antigen (PSMA), protein tyrosine kinase 7 (PTK7), solute carrier family 44 member 4 (SLC44A4), SLIT like family member 6 (SLITRK6), STEAP family member 1 (STEAP1), tissue factor (TF), T cell immunoglobulin and mucin protein-1 (TIM⁻¹), Tn antigen, trophoblast cell-surface antigen (TROP-2), Wilms' tumor 1 (WT1), or VEGF-A.

26. The mucin-active protease of any one of claims 19 to 25, wherein the mucin-active protease is stably associated with the targeting moiety via fusion of a protein domain comprising the mucin-active protease and a protein domain comprising the targeting moiety.

27. The mucin-active protease of claim 26, wherein the protein domain comprising the mucin-active protease is fused via a linker to the protein domain comprising the targeting moiety.

28. The mucin-active protease of claim 27, wherein the linker is a glycine-serine linker.

29. The mucin-active protease of any one of claims 19 to 25, wherein the mucin-active protease is stably associated with the targeting moiety via conjugation.

30. A nucleic acid encoding the mucin-active protease of any one of claims 1 to 29.

31. The nucleic acid of claim 30, wherein the mucin-active protease is stably associated with the targeting moiety via fusion of a protein domain comprising the mucin-active protease and a protein domain comprising the targeting moiety, and wherein the nucleic acid encodes the protein domain comprising the mucin-active protease fused to the protein domain comprising the targeting moiety.

32. A cell comprising the nucleic acid of claim 30 or claim 31.

33. A cell comprising an expression vector comprising the nucleic acid of claim 30 or claim 31 operably linked to a promoter.

34. A method of producing a mucin-active protease, the method comprising culturing the cell of claim 33 under conditions suitable for the cell to express the mucin-active protease, wherein the mucin-active protease is produced.

35. A composition comprising the mucin-active protease of any one of claims 1 to 29.

36. A pharmaceutical composition, comprising:
the mucin-active protease of any one of claims 1 to 29; and
a pharmaceutically acceptable carrier.

37. A method of treating a mucin-associated condition in a subject in need thereof, the method comprising:
administering to the subject an effective amount of the mucin-active protease of any one of claims 1 to 29,
wherein upon administration of the mucin-active protease to the subject, the targeting moiety targets the mucin-active protease to cell surface, extracellular and/or secreted mucins, and the mucin-active protease degrades the mucins.

38. The method according to claim 37, wherein the mucin-associated condition is a proliferative disorder.

39. The method according to claim 38, wherein the proliferative disorder is cancer.

40. The method according to claim 39, wherein the cancer comprises a solid tumor.

41. The method according to claim 40, wherein the solid tumor is a carcinoma or a sarcoma.

42. The method according to claim 41, wherein the carcinoma is a basal cell carcinoma, squamous cell carcinoma, renal cell carcinoma, ductal carcinoma in situ (DCIS), invasive ductal carcinoma, or adenocarcinoma.

43. The method according to any one of claims 40 to 42, wherein the solid tumor is immune-infiltrated.

44. The method according to claim 43, wherein the mucin-active protease affects the activation state of immune cells in the tumor microenvironment.

45. The method according to claim 44, wherein the mucin-active protease increases activation of tumor infiltrating immune cells.

46. The method according to claim 39, wherein the cancer is a myeloma, a leukemia, a lymphoma, or mixed type.

47. The method according to any one of claims 39 to 46, wherein the cancer is susceptible to mechanical stress.

48. The method according to any one of claims 39 to 47, wherein the cancer is sensitive to ferroptosis.

49. The method according to any one of claims 39 to 47, wherein the cancer is of a mucinous subtype.

50. The method according to claim 37, wherein the mucin-associated condition is a viral infection.

51. The method according to claim 50, wherein the viral infection is a respiratory virus infection.

52. The method according to claim 37, wherein the mucin-associated condition is cystic fibrosis.

53. The method according to claim 37, wherein the mucin-associated condition is bacterial endocarditis.

54. The method according to claim 37, wherein the mucin-associated condition is gut dysbiosis.


SEQUENCE LISTING (19 sequences)

SEQ ID NO: 1 - protein, 863 aa, synthetic construct
ADNNSAIYFNTSQPINDLQGSLAAEVKFAQSQILPAHPKEGDSQPHLTSLRKSLLLVRPVKADDKTPVQVEARDDNNKILGTLTLYPPSSLPDTIYHLDGVPEGGIDFTPHNGTKKIINTVAEVNKLSDASGSSIHSHLTNNALVEIHTANGRWVRDIYLPQGPDLEGKMVRFVSSAGYSSTVFYGDRKVTLSVGNTLLFKYVNGQWFRSGELENNRITYAQHIWSAELPAHWIVPGLNLVIKQGNLSGRLNDIKIGAPGELLLHTIDIGMLTTPRDRFDFAKDKEAHREYFQTIPVSRMIVNNYAPLHLKEVMLPTGELLTDMDPGNGGWHSGTMRQRIGKELVSHGIDNANYGLNSTAGLGENSHPYVVAQLAAHNSRGNYANGIQVHGGSGGGGIVTLDSTLGNEFSHEVGHNYGLGHYVDGFKGSVHRSAENNNSTWGWDGDKKRFIPNFYPSQTNEKSCLNNQCQEPFDGHKFGFDAMAGGSPFSAANRFTMYTPNSSAIIQRFFENKAVFDSRSSTGFSKWNADTQEMEPYEHTIDRAEQITASVNELSESKMAELMAEYAVVKVHMWNGNWTRNIYIPTASADNRGSILTINHEAGYNSYLFINGDEKVVSQGYKKSFVSDGQFWKERDVVDTREARKPEQFGVPVTTLVGYYDPEGTLSSYIYPAMYGAYGFTYSDDSQNLSDNDCQLQVDTKEGQLRFRLANHRANNTVMNKFHINVPTESQPTQATLVCNNKILDTKSLTPAPEGLTYTVNGQALPAKENEGCIVSVNSGKRYCLPVGQRSGYSLPDWIVGQEVYVDSGAKAKVLLSDWDNLSYNRIGEFVGNVNPADMKKVKAWNGQYLDFSKPRSMRVVYK

SEQ ID NO: 2 - protein, 664 aa, synthetic construct
ADNNSAIYFNTSQPINDLQGSLAAEVKFAQSQILPAHPKEGDSQPHLTSLRKSLLLVRPVKADDKTPVQVEARDDNNKILGTLTLYPPSSLPDTIYHLDGVPEGRSGELENNRITYAQHIWSAELPAHWIVPGLNLVIKQGNLSGRLNDIKIGAPGELLLHTIDIGMLTTPRDRFDFAKDKEAHREYFQTIPVSRMIVNNYAPLHLKEVMLPTGELLTDMDPGNGGAHSGTMRQRIGKELVSHGIDNANYGLNSTAGLGENSHPYVVAQLAAHNSRGNYANGIQVHGGSGGGGIVTLDSTLGNEFSHEVGHNYGLGHYVDGFKGSVHRSAENNNSTWGWDGDKKRFIPNFYPSQTNEKSCLNNQCQEPFDGHKFGFDAMAGGSPFSAANRFTMYTPNSSAIIQRFFENKAVFDSRSSTGFSKWNADTQEMEPYEHTIDRAEQITASVNELSESKMAELMAEYAVVKVHMWNGNWTRNIYIPTASADNRGSILTINHEAGYNSYLFINGDEKVVSQGYKKSFVSDGQFWKERDVVDTREARKPEQFGVPVTTLVGYYDPEGTLSSYIYPAMYGAYGFTYSDDSQNLSDNDCQLQVDTKEGQLRFRLANHRANNTVMNKFHINVPTESQPTQATLVCNNKILDTKSLTPAPEGLTYTVNGQALPAK

SEQ ID NO: 3 - protein, 1372 aa, synthetic construct
MNKVYSLKYCPVTGGLIAVSELARRVIKKTCRRLTHILLAGIPAICLCYSQISQAGIVRSDIAYQIYRDFAENKGLFVPGANDIPVYDKDGKLVGRLGKAPMADFSSVSSNGVATLVSPQYIVSVKHNGGYRSVSFGNGKNTYSLVDRNNHPSIDFHAPRLNKLVTEVIPSAVTSEGTKANAYKYTERYTAFYRVGSGTQYTKDKDGNLVKVAGGYAFKTGGTTGVPLISDATIVSNPGQTYNPVNGPLPDYGAPGDSGSPLFAYDKQQKKWVIVAVLRAYAGINGATNWWNVIPTDYLNQVMQDDFDAPVDFVSGLGPLNWTYDKTSGTGTLSQGSKNWTMHGQKDNDLNAGKNLVFSGQNGAIILKDSVTQGAGYLEFKDSYTVSAESGKTWTGAGIITDKGTNVTWKVNGVAGDNLHKLGEGTLTINGTGVNPGGLKTGDGIVVLNQQADTAGNIQAFSSVNLASGRPTVVLGDARQVNPDNISWGYRGGKLDLNGNAVTFTRLQAADYGAVITNNAQQKSQLLLDLKAQDTNVSEPTIGNISPFGGTGTPGNLYSMILNSQTRFYILKSASYGNTLWGNSLNDPAQWEFVGMDKNKAVQTVKDRILAGRAKQPVIFHGQLTGNMDVAIPQVPGGRKVIFDGSVNLPEGTLSQDSGTLIFQGHPVIHASISGSAPVSLNQKDWENRQFTMKTLSLKDADFHLSRNASLNSDIKSDNSHITLGSDRAFVDKNDGTGNYVIPEEGTSVPDTVNDRSQYEGNITLNHNSALDIGSRFTGGIDAYDSAVSITSPDVLLTAPGAFAGSSLTVHDGGHLTALNGLFSDGHIQAGKNGKITLSGTPVKDTANQYAPAVYLTDGYDLTGDNAALEITRGAHASGDIHASAASTVTIGSDTPAELASAETAASAFAGSLLEGYNAAFNGAITGGRADVSMHNALWTLGGDSAIHSLTVRNSRISSEGDRTFRTLTVNKLDATGSDFVLRTDLKNADKINVTEKATGSDNSLNVSFMNNPAQGQALNIPLVTAPAGTSAEMFKAGTRVTGFSRVTPTLHVDTSGGNTKWILDGFKAEADKAAAAKADSFMNAGYKNFMTEVNNLNKRMGDLRDTNGDAGAWARIMSGAGSADGGYSDNYTHVQVGFDKKHELDGVDLFTGVTMTYTDSSADSHAFSGKTKSVGGGLYASALFESGAYIDLIGKYIHHDNDYTGNFASLGTKHYNTHSWYAGAETGYRYHLTEDTFIEPQAELVYGAVSGKTFRWKDGDMDLSMKNRDFSPLVGRTGVELGKTFSGKDWSVTARAGTSWQFDLLNNGETVLRDASGEKRIKGEKDSRMLFNVGMNAQIKDNMRFGLEFEKSAFGKYNVDNAVNANFRYMF

SEQ ID NO: 4 - protein, 1687 aa, synthetic construct
MNKRKIAAIILATMITNLSATTIDVLAQELNTKNNSKVEVSHDDESHQARVSKFDLYNSDKLDAYNQEFQVSRSNIKSINNNGGKYNSSTIDKAIDGNLETHWETGKPNDANFTNEVVVTFNEITNIDRIVYSARRDSARGKGFAKEFEIYASLKDEGDDFNLVSSGEYTESTRDLVEIKFNPTDFKRLKFKFKKADQNWASAAEFMFYKEDKLNEKFNGLFTDSSMNKVSEEFNTLEKLNAFENELKDHPIYDLYKEGLNNARAILTETSENPTKATLGQITYNLNDDYNNQYRMPYKNIKAIKNNGRHYAAQNIEKAIDNDVNTYWETGTLNSSSFNNEVEVEFNDLVTLDRIVYGSRQSDLKGFAEEVYIYASRTSKGDTYKLVATGAHEATKGLVEAKFEPTEFKRVKFKFKKSKQNSATLNELMFYKPDEVYSSIPKLFTDGTMSELSEEFNSLEKINAFKEKAKNHPLYNDFNETIELAESLISNPRKEDVLELEMRGDSISEAKKRKVWNFQDWQITGLSARAGDKITVYVDVAEGDPTPTLLYKQSLTQHGGATSFQLKPGKNEITIPEINYESNGIPKDVIQGGDLFFTNYKSDSQKRAPKVRIEGASKYPVFILGKSDENEVMKELEAYVEKIKAEPKTTPNIFAVSSNKSLEFVQATYALDWYKKNNKTPKYTAEQWDQYIADAMGFWGFDNSKDVNSDFNFRIMPMVKNLSGGAFMNAGNGVIGIRPGNQDAILAANKGWGVAHELGHNFDTGGRTIVEVTNNMMPLFFESKYKTKTRITDQNIWENNTYPKVGLDDYSNNELYNKADSTHLAQLAPLWQLYLYDNTFYGKFERQFRERDFGNKNREDIYKSWVVAASDAMELDLTEFFARHGIRVDDKVKEDLAKYPKPDKKIYYLNDLAMNYKGDGFTENAKVSVSTSGSNGNIKLSFSVDDENKDNILGYEIRRDGKYVGFTSNDSFVDTKSNLDEDGVYVVTPYDRKLNTLNPIEVNALQPTLSVNPVITLALGEEFNEEEYIVAKDIKGNSLSESVKVKSSNVNTSKVGEYEVLYSLEDSKGNEYTKTSKVNVVSRKEYMSDLTPKQSSNGWGTVRKDKSISGGVIGLTRDGDFVDYNKGLGLHSNAEYVYDLEGKDYDYFESYVGVDKAMSSRPASSVIFKVLVDGEEKFNSGVMRSTTPQKYVKVDVKNAKELKLIVNDAGDGDSSDHASFGDAKLATLSSKPIIKGENLAYSMDEKVDLMKGITATDIEDGNITSKVQIKSSDFVEGKSGIFTVVYSVTDSDGLTSECSRTIAVTDKETQLSDLNWKSATIGSGSVRKDRAVSGNQIRLLNEDNSVETFAKGIGTHSYSEIVYNSEGYDIFDTWVGIDRHVADKKVSSVKFKVYVDGELKAETDVMRIDTPKKRLVVDVRNSKEIKLVVDVADNGNNWDHADWADAKFRNLAEYDASELNKAIEEAKKLDLNNYTEESSEALKNAISKGEEALLSKDKETINSALEELNKEMNSLVKVDLNAVINIPDKYLLKSIQNQLNKTGDITLGDMYSLTTLTLSGVEDLTGLENAKNLETLNMDYNEVKDLRPLSKLKKLNTLNAQEQFIAAGELKPSNGKVIGDSKVYNREGKNVAKTIRVVDKNGNTILEQDAKDEFTINTKDLSSGLYGVHVLFEDEGFSGVMFYLFNV

SEQ ID NO: 5 - protein, 1704 aa, synthetic construct
LSAYNSQLSIGVGEHLPEPLKIEGYQYIGYIKTKKQDNTELSRTVDGKYSAQRDSQPNSTKTSDVVHSADLEWNQGQGKVSLQGEASGDDGLSEKSSIAADNLSSNDSFASQVEQNPDHKGESVVRPTVPEQGNPVSATTVQSAEEEVLATTNDRPEYKLPLETKGTQEPGHEGEAAVREDLPVYTKPLETKGTQGPGHEGEAAVREEEPAYTEPLATKGTQEPGHEGKATVREETLEYTEPVATKGTQEPEHEGEAAVEEELPALEVTTRNRTEIQNIPYTTEEIQDPTLLKNRRKIERQGQAGTRTIQYEDYIVNGNVVETKEVSRTEVAPVNEVVKVGTLVKVKPTVEITNLTKVENKKSITVSYNLIDTTSAYVSAKTQVFHGDKLVKEVDIENPAKEQVISGLDYYTPYTVKTHLTYNLGENNEENTETSTQDFQLEYKKIEIKDIDSVELYGKENDRYRRYLSLSEAPTDTAKYFVKVKSDRFKEMYLPVKSITENTDGTYKVTVAVDQLVEEGTDGYKDDYTFTVAKSKAEQPGVYTSFKQLVTAMQSNLSGVYTLASDMTADEVSLGDKQTSYLTGAFTGSLIGSDGTKSYAIYDLKKPLFDTLNGATVRDLDIKTVSADSKENVAALAKAANSANINNVAVEGKISGAKSVAGLVASATNTVIENSSFTGKLIANHQDSNKNDTGGIVGNITGNSSRVNKVRVDALISTNARNNNQTAGGIVGRLENGALISNSVATGEIRNGQGYSRVGGIVGSTWQNGRVNNVVSNVDVGDGYVITGDQYAAADVKNASTSVDNRKADRFATKLSKDQIDAKVADYGITVTLDDTGQDLKRNLREVDYTRLNKAEAERKVAYSNIEKLMPFYNKDLVVHYGNKVATTDKLYTTELLDVVPMKDDEVVTDINNKKNSINKVMLHFKDNTVEYLDVTFKENFINSQVIEYNVTGKEYIFTPEAFVSDYTAITNNVLSDLQNVTLNSEATKKVLGAANDAALDNLYLDRQFEEVKANIAEHLRKVLAMDKSINTTGDGVVEYVSEKIKNNKEAFMLGLTYMNRWYDINYGKMNTKDLSTYKFDFNGNNETSTLDTIVALGNSGLDNLRASNTVGLYANKLASVKGEDSVFDFVEAYRKLFLPNKTNNEWFKENTKAYIVEMKSDIAEVREKQESPTADRKYSLGVYDRISAPSWGHKSMLLPLLTLPEESVYISSNMSTLAFGSYERYRDSVDGVILSGDALRTYVRNRVDIAAKRHRDHYDIWYNLLDSASKEKLFRSVIVYDGFNVKDETGRTYWARLTDKNIGSIKEFFGPVGKWYEYNSSAGAYANGSLTHFVLDRLLDAYGTSVYTHEMVHNSDSAIYFEGNGRREGLGAELYALGLLQSVDSVNSHILALNTLYKAEKDDLNRLHTYNPVERFDSDEALQSYMHGSYDVMYTLDAMEAKAILAQNNDVKKKWFRKIENYYVRDTRHNKDTHAGNKVRPLTDEEVANLTSLNSLIDNDIINRRSYDDSREYKRNGYYTISMFSPVYAALSNSKGAPGDIMFRKIAYELLAEKGYHKGFLPYVSNQYGAEAFASGSKTFSSWHGRDVALVTDDLVFKKVFNGEYSSWADFKKAMFKQRIDKQDNLKPITIQYELGNPNSTKEVTITTAAQMQQLINEAAAKDITNIDRATSHTPASWVHLLKQKIYNAYLRTTDDFRNSIYK

SEQ ID NO: 6 - protein, 834 aa, synthetic construct
KDTEKSIINSSFSISEEYLIQNLDKSSTSVQIPINTSMELAQWSVSYEANWLQCSKQKTAAEGTFLRITVNENTGETKRTANIKVTSTTATYTITVNQYAKGEVIVEGDIKVTPTGGKASEHQEGQDIENTYDGKFSTDGAAPFHTPWGQSAKFPVTLEYYFKGDTEIDYLIYYTRSGNGNFGKVKVYTTTNPDRSDYTLQGEYDFKEQNAPSKVSFSEGIKATGIKFEVLSGLGDFVSCDEMEFYKTNTDKTLDKQLLTVFTDITCTEIKNNVTNEQIQALPDYFVRIAEAVRDNTYDKWEKEFRIRSYEPYSNIAEWADKLMTKKYSDLDNPTGISVKAGDDIIVLVGDTYGQNISMQCIWETGTEYKQTASSGDVYMLNPGVNKLTMKGEGQLFVMYNTELTSNTAKPIKIHIPLGSGTVNGFFDLKEHKTDEKYAELLKKSTHKYFCIRGEKIMFYFHRNKLLEYVPNNILSAIHLWDNIVGWQQELMGIDDVRPSQVNNHLFAISPEGSYMWASDYQIGFVYTYLGNILLEDNVMAAEDNAWGPAHEIGHVHQAAINWASSTESSNNLFSNFIIYKLGKYKSRGNGLGSVATARYANGQAWYNMGDATHQNEDTETHMRMNWQLWIYYHRCEYKTDFWQTLFKLMREVNMTEGEDPGKKQLEFAKMASKAANQNLTDFFEMWGFFEPVNTTIEQYGTYKYYVSDAMIREAKEYMAQFPAPKHAFQYIEDRKKSEFPSNDYRYSAVGDVGYYTQFKENQKITKAITAELAGRKVSIQNGDEAVAFELRENDENGKLLYFSTFTTFEIPSSILMVNAKLYAVQADGKRILL

SEQ ID NO: 7 - protein, 486 aa, synthetic construct
ANTPEHIGNDLKLFKDSSCTSLKPDVKNTSAFQSDAMKELATKILAGHYKPDYLYAEYRALPSPRQTGKNLRIGDGFSKYDNMTGVYLEKGRHVVLVGKTEGQEISLLLPNLMRKPAEGVQPTKDPNGWGLHKKQIPLKEGINIIDVETPANAYISYFTEDAGKAPKIPVHFVTGKANGYFDTTRGDTNKDWVRLLDQAVSPIMDARGKYIQVAYPVEFLKKFTKDRGTELINAYDKLIGIQYQLMGLDKYGKIPENRVLARVNFNYYMFRDGDGVAYLGNDGTMRMVTDPENVLKGDACWGFSHEVGHVMQMRPMTWGGMTEVSNNIFSLQAAAKTGNESRLKRQGSYDKARKEIIEGEIAYLQSKDVFNKLVPLWQLHLYFTKNGHPDFYPDVMEYLRNNAGNYGGNDTVKYQFEFVKACCDVTKTDLTDFFEKWGFFKPGKFHIGDYAQYDFNVTPEMVEETKKWIAGKGYPKPETDITELSE

SEQ ID NO: 8 - protein, 720 aa, synthetic construct
KYPSLDVPEDILLHVKEAASSQSPYSGNHSVKQAVDGSMESANWHVPGVHHVEGEFVFEVPETIHYIIFSGANFNEIAVSAMSGSSWKDLGKFDIGGSRMIRFKKPLQKVRKIRLTVDYPEGSSPSFTVREISFYKRVESALNRKLLKVFKDTSCSSINPRCTLTDLKALPEFLQMIAKKIKSGDYEDKEFRIASYKAYSHPEFAAKVRNINALNKFDNPTGIVAEKGDEILVFVGPTHGEDIGLASVSPAGIESSSYPLNEGVNKIRINRSGLLYVMYHTDISPPKKPITVHIPVGSGIVNGYFDVTRHTDKDWKRMISNAPHSMFDIVGRNSMMILHTKYLKDYSPDSITKSVRVWDESVKAMWKIMGFDKYPQPHNNRQLGVSVEGGAHMFATWYYCGYSIGDQGNTLKNEVLAPGVLQGNRLWGIGHEIGHCYQHPFNWRSMSESSNNFFAQLILDQVTNAINGNEQASDMENPCKYLLSEAVKGMPFHDLNGWAKWGFAQYSFYLYFHKLGINPEFYPRLFESLRRKPLSRQAYEVSEAHLALYERICNISRTDFTDDFEIFNWFVPIDRKGHQYGDYSFKMTEEMARASKARIAAKRYPKPKFRIAFLHQHGKTVNLWGQNLHGSQLNGYWTKYKQNAKLSPSVSASKKDNMIIVRNGENAAAFCVVTNGKVVGYYDRQKFDVSGVEWNDTSKVYAIPIQTAEPYKLIYAAGRS

SEQ ID NO: 9 - protein, 612 aa, synthetic construct
QDNLAKRKAAQELTAERKLKEKKAREAAEKQRIKREREIREKKEKERLAAQKAYEEAQEEKARQAAEAARKLQEQAEREEREKRRREELERREREEEARRQEEDTPVEEEPEPEGRFPQPVKNRMPELSVYSIPCRDDIQTEKDKLLETWSWDKAEKMEGMEEFPTGSSPWKKGKDAGRMQALLEKCREWKDAKLASLKACPAAKDFPGVPENGAQTVRRTVEIDSNIGGWHSTGLYAPPGAEISCSLSGAPKDGSISVRIGCHTDSLHKLDEWKRVPEITMQVPAGRGRVKMVNPMGGLVYVNVGQRPRRGKVFKVQISGAVPSPLFVMGKTTPEQWAEQLENTKAPWGEIRMPRLIVTMPVEQLKQCPDVQKTAEFLQKNMALQDWIMGWDTKPDRLHHPMRFVVDRQISAGAGHSGYPAMATKDWTNSIATGSIIHSGSWGLWHELGHNHQSPPFTMEGQTEVSVNIFSMVCEVMGTGKDFESCWGGGMGPYGMSAEMKKYFSGTQTYNEAPNKVQLFFWVELMYYLGFDAFRQVALQFHDKPYDNGELSDEKKWEWVMNAFSKVTGKNMGPFFKIWRTPVSERATGRMKDLPAWLPSKDYPACYTAEE

SEQ ID NO: 10 - protein, 855 aa, synthetic construct
MPTITKTQSLYTLTRPVWLDAAGMSKGIDHDRQHLGIILAAGQVLRVRQTNAAFTGKLTLRLLNDDSKTEAEFSVGASWVEAQVNAVSVPFIDTPYGQGEPALEFEYPDTAKALPVYRRGENEAAFFARWDDRDAEFALVDAEYAVLLVPKISKPALKSLGEAKNIDGLIAYYDRIFTFYNALTGVSFEAEHESDRNIRNRYFMKADKHGAGAAYYGGRWTAETSSSVSSFWLTPKDSNWGSLHEIAHGYQGHFMGDKYFSTGEMWNNIYAACYQDVMLGERKYQEGWLYNYGNAAGVEKKIVDLIAQGTPLNQWDLRSKLFFAMLMVDKAGKNAFTHFNQQYRQSCNTPGFVPSEHALLDLLSASFAAAGEQIDVTPFVELTGGVITQTQRDLNAFSHAKAVYPLYQLVDAQSLPAVLAQLKLDSSLRLVDSSQLKASGLKGDVALSLDIDDFAQIYGEDLVLMEGARYAHKVRIDAPTLALNALPIGVYTLRLPTGKDRKYRPTAGYLTVKQGKSAVTLRFERKAASPIVSQEINLLGLSDAVFATVVVDRAKGAVVVDVTSTTPHAYFPGETYARVTLRDRQGAVRFSKEVQGTQATLSHDELPFAEGDDIEIYHVEPSRVRLNPAAAGVIDNKNKTNVLRIETSGLKNPALSGDPEQALLMRLEQAAQRLRRLLPAYYAPSSSFKDDIYLAIGAFAEPQREPLLATYRDCLPADNTPPGANVGNAFTVAGKGIGDRQFLTCAVDLVKKTVTVTLAAGIAHHYFADSYAFLSVTDADGNTLLSYDVIGSQNQQAKTWVLPLSGYGGEVIHLRHEEPDNRLTIVNEMQHVRLAEKGKQQAYCITPVGLKRIKD

SEQ ID NO: 11 - protein, 961 aa, synthetic construct
QVIQFNNETPVNNLQGDFEAMVIYAQNITVPHYNDKGDPRPHLTALRDTLLLVKPVEPLNVSSQVKVIARDSQGNQLGALSLNTPEQLPNHDGPNLDIMYAKDMWSVTLPATWIQPGLTLELLNGSTSGLLSDLDIGAPNELLINTMDLGMLTNPRGRFYFADNPELHKDYFQKIPVSKLIVNPYETVKLDEVMLPDGDLLTELDPSTGTWHQGDMRADTTKILMSHGINLANYGINSSTSVSERAHPYTANQITAIAAVGRYQNGVVAHGGSGGNGMVTIDSSVGNEWSHEVGHNFGLGHWPGGTDGTTHRPSTDINSAWGWDLFQKRFIANFMWNKRNGEDQVCCSDGIGIPAFEGYKFNRDAMGGGEPTSPISKYTLHTPYVLEKIQTFMEGKATFDESSPTGFTKWNDETKAMQAFQQPAILLSKSITSQSQLNTIKDDIDGSVLLGYINDFDITKVETGDGRWIRDIYLPSAENVAVGKAVNIARYSGYGVTAHVNGQLVNLNRGDSKFYISDGKAWLETTEDQVAEGKPTRIPTDYGVPVTTLVGYYDPQQQLDSYIFPALHGAYGFVYQPTPVDRLDTTGCYVKVYNGQDHQTDNYQLVGFRFDDNVMNKFHINLKQTDDPTRAEVVCDNAVLSSLDIEKPKQALEVSIVQSDSLKPSENNKPVAHAGEDQSVLSGATITLSADKSTDADGDKLTYVWEQISGLPASIQASNAMTTNVVLAESTQEQSYVFSVLVSDGKSSSSDMVTIIAQPQPTQNHAPEVSLPSSIDAKPGEVIEITATASDPDGDVLSFKWSTSGLAYQTMSGGTIQLSVPDVTVDSQFLLSVVVSDPKGESASANTTLNVKASGNTCSVSDPNATNYDAWSASKSYSGGDLVSYKQLVWKAKHWSQNNQPDSSDAWELLSDVVLPWSSQAAYSGGAQVTYNGVKFEAKWWTRGEQPNVSSVWINKGAACQ

SEQ ID NO: 12 - protein, 593 aa, synthetic construct
MNFKLKTSLIIGAIVASSLVYAATVLSPNQNNNSGSIPSGYSDLEFNLANGNWVKNLTLPTSANNLDKITIRSSAAYSSYLDTSNTNIPLEVLKINSGDVYQFIFNSSQNKWIAQLATVSPTNGATYEVVPLTTASMQKVLIQNEKWAQTIALPSDVRDGTTVQVVSTASASSDIDKTNLLFPSSFILKNGSEYWFKYYSALGKWVPEYIKPQKLNVQQIGTSLATVNSPLTEVSFGDGNWVSNFTLPTTASDRDKIIIKSTATWSAKINNTNINSQATLTLKTGDQYEFMYVSDKGYWQLISSPTKVIDSAATIPATLPNMTQPTLKVKLSTSNWQPTLQLPAKAQVGDKVVIVSNASADTYINAANGLSTAIKNGENRRFIYTAQGWTVDSYTIDMLLVSSPEVNSILGESAAKLRMIEGVNLTNLTAENSNARFYLRNVGYLTYKIPAATLKEAISTGRDDTTVQNERKRVLADGVYYQGNEPGDGGCGWAWINASAYNMIGANDIAGCSFAAMRHEVGHNLGLYHNGSTNIGSGFAHPLGSTAMGGNNINFYSSPYLYNPKYGVRLGVEGKIDAVSVINLNAQKISLYN

SEQ ID NO: 13 - protein, 923 aa, synthetic construct
MSLSTTAFPSLQGENMSRSPIPRHRALLAGFCLAGALSAQAATQEEILDAALVSGDSSQLTDSHLVALRLQQQVERIRQTRTQLLDGLYQNLSQAYDPGAASMWVLPANPDNTLPFLIGDKGRVLASLSLEAGGRGLAYGTNVLTQLSGTNAAHAPLLKRAVQWLVNGDPGAATAKDFKVSVVGVDKTAALNGLKSAGLQPADAACNALTDASCASTSKLLVLGNGASAASLSATVRARLQAGLPILFVHTNGWNQSSTGQQILAGLGLQEGPYGGNYWDKDRVPSSRTRTRSVELGGAYGQDPALVQQIVDGSWRTDYDWSKCTSYVGRTTCDDVPGLSDFSKRVDVLKGALDAYNQKAQNLFALPGTTSLRLWLLWADAVRQNIRYPMDKAADTARFQETFVADAIVGYVREAGAAQKELGSYAGQRQQSMPVSGSEETLTLTLPSAQGFTAIGRMAAPGKRLSIRIEDAGQASLAVGLNTQRIGSTRLWNTRQYDRPRFLKSPDIKLQANQSVALVSPYGGLLQLVYSGATPGQTVTVKVTGAASQPFLDIQPGEDSSQAIADFIQALDADKADWLEIRSGSVEVHAKVEKVRGSIDKDYGGDVQRFIRELNEVFIDDAYTLAGFAIPNQAKTPAIQQECAARGWDCDSETLHKLPGTQHINVDQYAQCGGGCSGNPYDQTWGLNPRGWGESHELGHNLQVNRLKVYGGRSGEISNQIFPLHKDWRVLREFGQNLDDTRVNYRNAYNLIVAGRAEADPLAGVYKRLWEDPGTYALNGERMAFYTQWVHYWADLKNDPLQGWDIWTLLYLHQRQVDKSDWDANKAALGYGTYAQRPGNSGDASSTDGNDNLLLGLSWLTQRDQRPTFALWGIRTSAAAQAQVAAYGFAEQPAFFYANNRTNEYSTVKLLDMSQGSPAWPFP

SEQ ID NO: 14 - protein, 385 aa, synthetic construct
MLKRLLSAFFSLFFLGAASGTSFAEVTVPDALKDRIALKKTARQLNIVYFLGSDTEPVPDYERRLSELLLYLQQFYGKEMQRHGYGARSFGLDIKSPGRVNIIEYKAKNPAAHYPYENGGGWKAAQELDEFFKAHPDRKKSQHTLIIMPTWNDEKNGPDNPGGVPFYGMGRNCFALDYPAFDIKHLGQKTREGRLLTKWYGGMAHELGHGLNLPHNHQTASDGKKYGTALMGSGNYTFGTSPTFLTPASCALLDACEVFSVTPSQQFYEGKPEVEVGDVAISFKGDQILVSGNYKSPQTVKALNVYIQDPPYAVNQDYDAVSFSRRLGKKSGKFSMKIDKKELEGLNNNEFRISLMFILANGLHMQKHFTFHWDALQDYRDGSKS

SEQ ID NO: 15 - protein, 817 aa, synthetic construct
MGSSHHHHHHSSGLVPRGSHMASEVQLVESGGGLVQAGGSLRLSCAASGITFSINTMGWYRQAPGKQRELVALISSIGDTYYADSVKGRFTISRDNAKNTVYLQMNSLKPEDTAVYYCKRFRTAAQGTDYWGQGTQVTVSSGGSGGSGGSGGSADNNSAIYFNTSQPINDLQGSLAAEVKFAQSQILPAHPKEGDSQPHLTSLRKSLLLVRPVKADDKTPVQVEARDDNNKILGTLTLYPPSSLPDTIYHLDGVPEGRSGELENNRITYAQHIWSAELPAHWIVPGLNLVIKQGNLSGRLNDIKIGAPGELLLHTIDIGMLTTPRDRFDFAKDKEAHREYFQTIPVSRMIVNNYAPLHLKEVMLPTGELLTDMDPGNGGAHSGTMRQRIGKELVSHGIDNANYGLNSTAGLGENSHPYVVAQLAAHNSRGNYANGIQVHGGSGGGGIVTLDSTLGNEFSHEVGHNYGLGHYVDGFKGSVHRSAENNNSTWGWDGDKKRFIPNFYPSQTNEKSCLNNQCQEPFDGHKFGFDAMAGGSPFSAANRFTMYTPNSSAIIQRFFENKAVFDSRSSTGFSKWNADTQEMEPYEHTIDRAEQITASVNELSESKMAELMAEYAVVKVHMWNGNWTRNIYIPTASADNRGSILTINHEAGYNSYLFINGDEKVVSQGYKKSFVSDGQFWKERDVVDTREARKPEQFGVPVTTLVGYYDPEGTLSSYIYPAMYGAYGFTYSDDSQNLSDNDCQLQVDTKEGQLRFRLANHRANNTVMNKFHINVPTESQPTQATLVCNNKILDTKSLTPAPEGLTYTVNGQALPAK

SEQ ID NO: 16 - protein, 24 aa, synthetic construct
ETSSVKQPWDHTTTTTTNRPGTTR

SEQ ID NO: 17 - protein, 72 aa, synthetic construct
KSLTSKVPPTVQKPTTVNVPTTEVSPTSQKTTTKTTTPNAQATRSTPVSRTTKHFHETTPNKGSGTTSGTTR

SEQ ID NO: 18 - protein, 33 aa, synthetic construct
PPSSTKPPALSHSVSTSSTTKSPASSASGPRPT

SEQ ID NO: 19 - protein, 51 aa, synthetic construct
CTSTSPTRSMAPGAVHLPQPVSTRSQHTQPTPEPSTAPSTSFLLPMGPSPP